SEQUENCE LISTING (78 sequences)

SEQ ID NO: 1 - DNA, 40 bp, synthetic construct
  source 1..40: mol_type = other DNA
gatacagtgcacatgtggtacagttgaagtcggaagttta

SEQ ID NO: 2 - DNA, 41 bp, synthetic construct
  source 1..41: mol_type = other DNA
atctcaaaatagtaaatgctacagttgaagtcggaagttta

SEQ ID NO: 3 - DNA, 41 bp, synthetic construct
  source 1..41: mol_type = other DNA
aggtgactgataccaaaaatacagttgaagtcggaagttta

SEQ ID NO: 4 - DNA, 43 bp, synthetic construct
  source 1..43: mol_type = other DNA
atagtacaaagagttctcatatacagttgaagtcggaagttta

SEQ ID NO: 5 - DNA, 42 bp, synthetic construct
  source 1..42: mol_type = other DNA
agacagacctacaaagaatatacagttgaagtcggaagttta

SEQ ID NO: 6 - DNA, 42 bp, synthetic construct
  source 1..42: mol_type = other DNA
gcaaaccaaaatggcacatgtacagttgaagtcggaagttta

SEQ ID NO: 7 - DNA, 43 bp, synthetic construct
  source 1..43: mol_type = other DNA
tattatcaatagcacctaatctacagttgaagtcggaagttta

SEQ ID NO: 8 - DNA, 43 bp, synthetic construct
  source 1..43: mol_type = other DNA
aaatttctagaaaagggttggtacagttgaagtcggaagttta

SEQ ID NO: 9 - DNA, 43 bp, synthetic construct
  source 1..43: mol_type = other DNA
aatgattatggcattcatatatacagttgaagtcggaagttta

SEQ ID NO: 10 - DNA, 43 bp, synthetic construct
  source 1..43: mol_type = other DNA
ccagacttggtggcacacacctacagttgaagtcggaagttta

SEQ ID NO: 11 - DNA, 44 bp, synthetic construct
  source 1..44: mol_type = other DNA
aagagcttttatttacatgaactacagttgaagtcggaagttta

SEQ ID NO: 12 - DNA, 44 bp, synthetic construct
  source 1..44: mol_type = other DNA
cggaacgtgtaggttcgttacatacagttgaagtcggaagttta

SEQ ID NO: 13 - DNA, 45 bp, synthetic construct
  source 1..45: mol_type = other DNA
aatcctagaactggaaaatatagtacagttgaagtcggaagttta

SEQ ID NO: 14 - DNA, 46 bp, synthetic construct
  source 1..46: mol_type = other DNA
ctgtgagtgtggactgatcaaatatacagttgaagtcggaagttta

SEQ ID NO: 15 - DNA, 45 bp, synthetic construct
  source 1..45: mol_type = other DNA
atgactgtgtctgcacctctatctacagttgaagtcggaagttta

SEQ ID NO: 16 - DNA, 46 bp, synthetic construct
  source 1..46: mol_type = other DNA
agaccccatatctcacaccatacatacagttgaagtcggaagttta

SEQ ID NO: 17 - DNA, 46 bp, synthetic construct; identical to SEQ ID NO: 16
  source 1..46: mol_type = other DNA

SEQ ID NO: 18 - DNA, 45 bp, synthetic construct
  source 1..45: mol_type = other DNA
caagacctaggccatgcaagacatacagttgaagtcggaagttta

SEQ ID NO: 19 - DNA, 46 bp, synthetic construct
  source 1..46: mol_type = other DNA
ggaatctctttttctaattattgctacagttgaagtcggaagttta

SEQ ID NO: 20 - DNA, 18 bp, synthetic construct
  source 1..18: mol_type = other DNA
ccgcacgcgttctagact

SEQ ID NO: 21 - DNA, 28 bp, synthetic construct
  source 1..28: mol_type = other DNA
acaaagtagatgtcctaactgacttgcc

SEQ ID NO: 22 - DNA, 19 bp, synthetic construct
  source 1..19: mol_type = other DNA
gccgctcggtccgcacgtg

SEQ ID NO: 23 - DNA, 33 bp, synthetic construct
  source 1..33: mol_type = other DNA
agtgagtttaaatgtatttggctaaggtgtatg

SEQ ID NO: 24 - DNA, 20 bp, synthetic construct
  source 1..20: mol_type = other DNA
tatagtctagaacgcgtgcg

SEQ ID NO: 25 - DNA, 1458 bp, synthetic construct
  source 1..1458: mol_type = other DNA
atgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaggtaccaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 26 - protein, 486 aa, synthetic construct
MLLLVTSLLLCELPHPAFLLIPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAGTTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 227 bp, synthetic construct
  source 1..227: mol_type = other DNA
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 28 - DNA, 228 bp, synthetic construct
  source 1..228: mol_type = other DNA
ttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 29 - DNA, 141 bp, synthetic construct
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 30 - DNA, 141 bp, synthetic construct
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 31 - DNA, 1614 bp, synthetic construct
  source 1..1614: mol_type = other DNA
atgctgctgctggtgacaagcctgctgctgtgcgagctgccccaccccgccttcctgctgatccccgaggtgcagctgcagcagagcggcgccgagctggtgaagcccggcgctagcgtgaagatgagctgcaaggctagcggctacaccttcacaagctataacatgcactgggtgaagcagacccccggccaaggcctggaatggatcggtgccatctaccccggcaacggcgacacaagctataaccaaaaattcaagggcaaggccaccctgaccgccgacaagagcagcagcaccgcctacatgcagctgagcagcctgacaagcgaggacagcgccgactactactgcgcaagaagcaactactacggcagcagctactggttctttgacgtgtggggcgctggcaccacggtcaccgtatcatctggcgggggcggaagcggcggtggaggcagcggcggtggcggttctgacatcgtgctgacacagagccccgccatcttgagcgctagccccggcgagaaggtgaccatgacctgcagagctagctcatcggtgaactacatggactggtatcagaagaaacccggcagcagcccaaagccttggatctacgccacgagcaatctggcgagcggggtacccgcccggttctccggaagcggctcgggcacatcatactccctgaccataagccgtgtggaggccgaggacgccgccacctactactgtcagcagtggagcttcaaccctccgacctttggcggcggcactaaattagagattaagggcgggggaggtagtggcggcgggggcagcggcggtggtgggtccggaggagggggaagcggcggcgggggctcagatatacagatgacacagaccacaagcagcctgagcgcaagcctgggcgacagagtgaccatcagctgtagagcctcgcaagacatcagcaagtacctgaactggtatcagcagaagcccgacggcaccgtgaagctgctgatctaccacacaagcagactgcacagcggcgtgcctagccggttcagcggcagcggcagcggcaccgattactcgctgaccatcagtaatctcgagcaagaggacatcgccacctacttctgtcagcaaggcaacaccctgccctacacattcggcggcggtaccaagctggaaataacgggctcaacaagcggctccggtaagcccggcagtggcgagggcagtaccaagggagaggtgaagttacaagagagtggccccggcttagtcgccccaagccaaagcctgagcgtgacctgcaccgtgagcggcgtgagcctgcccgactacggcgtgagctggatcagacagccccctagaaaaggactcgagtggctgggcgtgatctggggcagcgagaccacctactacaacagcgccctgaagagcagactgaccatcatcaaggacaacagcaagagccaagtgttcctgaagatgaacagcctgcagaccgacgacaccgccatctactattgtgccaagcactactactacggcggcagctacgccatggattattggggccaaggaacctcagtaacggtcagcagcgcggctgcc

SEQ ID NO: 32 - protein, 265 aa, synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 33 - DNA, 1023 bp, synthetic construct
  source 1..1023: mol_type = other DNA
atgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggttttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactag

SEQ ID NO: 34 - RNA, 1023 nt, synthetic construct
  source 1..1023: mol_type = other RNA
atgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggttttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactag

SEQ ID NO: 35 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 36 - DNA, 6363 bp, synthetic construct
  source 1..6363: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatgggaaaatcaaaagaaatcagccaagacctcagaaaaagaattgtagacctccacaagtctggttcatccttgggagcaatttccaaacgcctggcggtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccactcagcaaggaagaagccactgctccaaaaccgacataagaaagccagactacggtttgcaactgcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatggacgcggtgcagtatgtggatatattgaagcaacatctcaagacatcagtcaggaagttaaagcttggtcgcaaatgggttttccaacacgacaatgaccccaagcatacttccaaagttgtggcaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaaattattgtgggaagcttgtggaaggctacccgaaacgtttgacccaagttaaacaatttaaaggcaatgctaccaaatactagaagcttaagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 37 - DNA, 3162 bp, synthetic construct
  source 1..3162: mol_type = other DNA
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagagaggatcctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatgctgctgctggtgacaagcctgctgctgtgcgagctgccccaccccgccttcctgctgatccccgaggtgcagctgcagcagagcggcgccgagctggtgaagcccggcgctagcgtgaagatgagctgcaaggctagcggctacaccttcacaagctataacatgcactgggtgaagcagacccccggccaaggcctggaatggatcggtgccatctaccccggcaacggcgacacaagctataaccaaaaattcaagggcaaggccaccctgaccgccgacaagagcagcagcaccgcctacatgcagctgagcagcctgacaagcgaggacagcgccgactactactgcgcaagaagcaactactacggcagcagctactggttctttgacgtgtggggcgctggcaccacggtcaccgtatcatctggcgggggcggaagcggcggtggaggcagcggcggtggcggttctgacatcgtgctgacacagagccccgccatcttgagcgctagccccggcgagaaggtgaccatgacctgcagagctagctcatcggtgaactacatggactggtatcagaagaaacccggcagcagcccaaagccttggatctacgccacgagcaatctggcgagcggggtacccgcccggttctccggaagcggctcgggcacatcatactccctgaccataagccgtgtggaggccgaggacgccgccacctactactgtcagcagtggagcttcaaccctccgacctttggcggcggcactaaattagagattaagggcgggggaggtagtggcggcgggggcagcggcggtggtgggtccggaggagggggaagcggcggcgggggctcagatatacagatgacacagaccacaagcagcctgagcgcaagcctgggcgacagagtgaccatcagctgtagagcctcgcaagacatcagcaagtacctgaactggtatcagcagaagcccgacggcaccgtgaagctgctgatctaccacacaagcagactgcacagcggcgtgcctagccggttcagcggcagcggcagcggcaccgattactcgctgaccatcagtaatctcgagcaagaggacatcgccacctacttctgtcagcaaggcaacaccctgccctacacattcggcggcggtaccaagctggaaataacgggctcaacaagcggctccggtaagcccggcagtggcgagggcagtaccaagggagaggtgaagttacaagagagtggccccggcttagtcgccccaagccaaagcctgagcgtgacctgcaccgtgagcggcgtgagcctgcccgactacggcgtgagctggatcagacagccccctagaaaaggactcgagtggctgggcgtgatctggggcagcgagaccacctactacaacagcgccctgaagagcagactgaccatcatcaaggacaacagcaagagccaagtgttcctgaagatgaacagcctgcagaccgacgacaccgccatctactattgtgccaagcactactactacggcggcagctacgccatggattattggggccaaggaacctcagtaacggtcagcagcgcggctgccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaggctagcaataaaagatctttattttcattagatctgtgtgttggttttttgtgtgaattcttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 38 - DNA, 3621 bp, synthetic construct
  source 1..3621: mol_type = other DNA
cagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagagaggatcctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatgcttctgctcgtgacaagcctgctgctgtgcgagctgccccaccctgcctttctgctgatccctcaggtgcagctgcagcagtctggccctggcctcgtgaagcctagccagaccctgagcctgacctgtgccatcagcggcgatagcgtgtccagcaatagcgccgcctggaactggatcagacagagccctagcagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggaggcggcggaagcgacatccagatgacacagagccccagctccctgagcgccagcgtgggagacagagtgaccatcacctgtcgggccagccagaccatctggtcctacctgaactggtatcagcagcggcctggcaaggcccccaacctgctgatctatgccgccagctcactgcagagcggcgtgcccagcagattttccggcagaggcagcggcaccgacttcaccctgacaatcagttccctgcaggccgaggacttcgccacctactactgccagcagagctacagcatcccccagaccttcggccaggggaccaagctggaaatcaaagcggccgcaggtaccaccacgacgccagcgccgcgaccaccaacaccggcgcccaccatcgcgtcgcagcccctgtccctgcgcccagaggcatgccggccagcagcagggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggcagtggagagggcagaggaagtctgctaacatgcggtgacgtcgaggagaatcctggcccactcgagggagtgcaggtggagactatctccccaggagacgggcgcaccttccccaagcgcggccagacctgcgtggtgcactacaccgggatgcttgaagatggaaagaaagttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaatctggcggtggatccggagtcgacggatttggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagtcgactatccgtacgacgtaccagactacgcactcgactaagctagcaataaaagatctttattttcattagatctgtgtgttggttttttgtgtgaattcttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 39 - DNA, 744 bp, synthetic construct
  source 1..744: mol_type = other DNA
gatatacagatgacacagaccacaagcagcctgagcgcaagcctgggcgacagagtgaccatcagctgtagagcctcgcaagacatcagcaagtacctgaactggtatcagcagaagcccgacggcaccgtgaagctgctgatctaccacacaagcagactgcacagcggcgtgcctagccggttcagcggcagcggcagcggcaccgattactcgctgaccatcagtaatctcgagcaagaggacatcgccacctacttctgtcagcaaggcaacaccctgccctacacattcggcggcggtaccaagctggaaataacgggctcaacaagcggctccggtaagcccggcagtggcgagggcagtaccaagggagaggtgaagttacaagagagtggccccggcttagtcgccccaagccaaagcctgagcgtgacctgcaccgtgagcggcgtgagcctgcccgactacggcgtgagctggatcagacagccccctagaaaaggactcgagtggctgggcgtgatctggggcagcgagaccacctactacaacagcgccctgaagagcagactgaccatcatcaaggacaacagcaagagccaagtgttcctgaagatgaacagcctgcagaccgacgacaccgccatctactattgtgccaagcactactactacggcggcagctacgccatggattattggggccaaggaacctcagtaacggtcagcagcgcggctgcc

SEQ ID NO: 40 - protein, 248 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAA

SEQ ID NO: 41 - DNA, 795 bp, synthetic construct
  source 1..795: mol_type = other DNA
atgctgctgctggtgacaagcctgctgctgtgcgagctgccccaccccgccttcctgctgatccccgaggtgcagctgcagcagagcggcgccgagctggtgaagcccggcgctagcgtgaagatgagctgcaaggctagcggctacaccttcacaagctataacatgcactgggtgaagcagacccccggccaaggcctggaatggatcggtgccatctaccccggcaacggcgacacaagctataaccaaaaattcaagggcaaggccaccctgaccgccgacaagagcagcagcaccgcctacatgcagctgagcagcctgacaagcgaggacagcgccgactactactgcgcaagaagcaactactacggcagcagctactggttctttgacgtgtggggcgctggcaccacggtcaccgtatcatctggcgggggcggaagcggcggtggaggcagcggcggtggcggttctgacatcgtgctgacacagagccccgccatcttgagcgctagccccggcgagaaggtgaccatgacctgcagagctagctcatcggtgaactacatggactggtatcagaagaaacccggcagcagcccaaagccttggatctacgccacgagcaatctggcgagcggggtacccgcccggttctccggaagcggctcgggcacatcatactccctgaccataagccgtgtggaggccgaggacgccgccacctactactgtcagcagtggagcttcaaccctccgacctttggcggcggcactaaattagagattaag

SEQ ID NO: 42 - protein, 265 aa, synthetic construct; identical to SEQ ID NO: 32

SEQ ID NO: 43 - DNA, 3754 bp, synthetic construct
  source 1..3754: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgttctagactatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattccagaaaatgatgtcatggctttagagaggatcctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagacgtacggccaccatgggaattcttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtataggcatgcggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 44 - DNA, 29 bp, synthetic construct
  source 1..29: mol_type = other DNA
ctcgtttttcaactactccacaaatttct

SEQ ID NO: 45 - DNA, 26 bp, synthetic construct
  source 1..26: mol_type = other DNA
gtgtcatgcacaaagtagatgtccta

SEQ ID NO: 46 - DNA, 35 bp, synthetic construct
  source 1..35: mol_type = other DNA
gctgaaatgaatcattctctctactattattctga

SEQ ID NO: 47 - DNA, 26 bp, synthetic construct
  source 1..26: mol_type = other DNA
aattccctgtcttaggtcagttagga

SEQ ID NO: 48 - DNA, 21 bp, synthetic construct
  source 1..21: mol_type = other DNA
agctgagtgcgtcctgtcact

SEQ ID NO: 49 - DNA, 22 bp, synthetic construct
  source 1..22: mol_type = other DNA
tctggccctagtctcagacctt

SEQ ID NO: 50 - DNA, 30 bp, synthetic construct
  source 1..30: mol_type = other DNA
tttgttaacaagaaatttgtggagtagttg

SEQ ID NO: 51 - DNA, 54 bp, synthetic construct
  source 1..54: mol_type = other DNA
aatgatacggcgaccaccgagatctacacaaaaacgagttttaatgactccaac

SEQ ID NO: 52 - DNA, 24 bp, synthetic construct
  source 1..24: mol_type = other DNA
aatgatacggcgaccaccgagatc

SEQ ID NO: 53 - DNA, 24 bp, synthetic construct
  source 1..24: mol_type = other DNA
caagcagaagacggcatacgagat

SEQ ID NO: 54 - DNA, 33 bp, synthetic construct
  source 1..33: mol_type = other DNA
taatacgactcactataggtgacagcgagcgct

SEQ ID NO: 55 - DNA, 33 bp, synthetic construct
  source 1..33: mol_type = other DNA
agcgctcgctgtcacctatagtgagtcgtatta

SEQ ID NO: 56 - DNA, 46 bp, synthetic construct
  source 1..46: mol_type = other DNA
aaaaacgagttttaatgactccaacttaagtgtatgtaaacttccg

SEQ ID NO: 57 - DNA, 52 bp, synthetic construct
  source 1..52: mol_type = other DNA
gcgctcgctgtcacctatagtgagtcgtattataatttttttttcaaaaaaa

SEQ ID NO: 58 - DNA, 54 bp, synthetic construct
  source 1..54: mol_type = other DNA
gttcccatggtactactcatataatacgactcactataggtgacagcgagcgct

SEQ ID NO: 59 - DNA, 21 bp, synthetic construct
  source 1..21: mol_type = other DNA
gttcccatggtactactcata

SEQ ID NO: 60 - DNA, 55 bp, synthetic construct
  source 1..55: mol_type = other DNA
caagcagaagacggcatacgagatcggtacaagctataatacgactcactatagg

SEQ ID NO: 61 - DNA, 55 bp, synthetic construct
  source 1..55: mol_type = other DNA
caagcagaagacggcatacgagatcggtaaacatcgtaatacgactcactatagg

SEQ ID NO: 62 - DNA, 55 bp, synthetic construct
  source 1..55: mol_type = other DNA
caagcagaagacggcatacgagatcggtacattggctaatacgactcactatagg

SEQ ID NO: 63 - DNA, 55 bp, synthetic construct
  source 1..55: mol_type = other DNA
caagcagaagacggcatacgagatcggtaccactgttaatacgactcactatagg

SEQ ID NO: 64 - DNA, 55 bp, synthetic construct
  source 1..55: mol_type = other DNA
caagcagaagacggcatacgagatcggtaacgtgattaatacgactcactatagg

SEQ ID NO: 65 - DNA, 55 bp, synthetic construct
  source 1..55: mol_type = other DNA
caagcagaagacggcatacgagatcggtcgctgatctaatacgactcactatagg

SEQ ID NO: 66 - DNA, 55 bp, synthetic construct
  source 1..55: mol_type = other DNA
caagcagaagacggcatacgagatcggtcagatctgtaatacgactcactatagg

SEQ ID NO: 67 - DNA, 55 bp, synthetic construct
  source 1..55: mol_type = other DNA
caagcagaagacggcatacgagatcggtatgcctaataatacgactcactatagg

SEQ ID NO: 68 - DNA, 55 bp, synthetic construct
  source 1..55: mol_type = other DNA
caagcagaagacggcatacgagatcggtctgtagcctaatacgactcactatagg

SEQ ID NO: 69 - DNA, 55 bp, synthetic construct
  source 1..55: mol_type = other DNA
caagcagaagacggcatacgagatcggtagtacaagtaatacgactcactatagg

SEQ ID NO: 70 - DNA, 55 bp, synthetic construct
  source 1..55: mol_type = other DNA
caagcagaagacggcatacgagatcggtcatcaagttaatacgactcactatagg

SEQ ID NO: 71 - DNA, 55 bp, synthetic construct
  source 1..55: mol_type = other DNA
caagcagaagacggcatacgagatcggtagtggtcataatacgactcactatagg

SEQ ID NO: 72 - DNA, 55 bp, synthetic construct
  source 1..55: mol_type = other DNA
caagcagaagacggcatacgagatcggtaacaaccataatacgactcactatagg

SEQ ID NO: 73 - DNA, 55 bp, synthetic construct
  source 1..55: mol_type = other DNA
caagcagaagacggcatacgagatcggtaaccgagataatacgactcactatagg

SEQ ID NO: 74 - DNA, 55 bp, synthetic construct
  source 1..55: mol_type = other DNA
caagcagaagacggcatacgagatcggtaacgcttataatacgactcactatagg

SEQ ID NO: 75 - DNA, 55 bp, synthetic construct
  source 1..55: mol_type = other DNA
caagcagaagacggcatacgagatcggtaagacggataatacgactcactatagg

SEQ ID NO: 76 - DNA, 55 bp, synthetic construct
  source 1..55: mol_type = other DNA
caagcagaagacggcatacgagatcggtaaggtacataatacgactcactatagg

SEQ ID NO: 77 - DNA, 55 bp, synthetic construct
  source 1..55: mol_type = other DNA
caagcagaagacggcatacgagatcggtacacagaataatacgactcactatagg

SEQ ID NO: 78 - DNA, 10 bp, synthetic construct
  source 1..10: mol_type = other DNA
acttcaactg

(19) United States
(12) Patent Application Publication
(10) Pub. No.: US 2025/0228940 A1
(43) Pub. Date: Jul. 17, 2025

(54) COMPOSITIONS AND METHODS FOR EFFICIENT AND STABLE GENETIC MODIFICATION OF EUKARYOTIC CELLS

(71) Applicant: Yale University, New Haven, CT (US)

(72) Inventors: Sidi Chen, New Haven, CT (US); Lupeng Ye, New Haven, CT (US); Stanley Lam, New Haven, CT (US)

(21) Appl. No.: 18/846,189

(22) PCT Filed: Mar. 13, 2023
(86) PCT No.: PCT/US2023/064244

§ 371 Date: Sep. 11, 2024

Related U.S. Application Data
(60) Provisional application No. 63/319,070, filed on Mar. 11, 2022.

(51) Int. Cl. A61K 40/11; A61K 40/31; A61K 40/42; C07K 16/28; C12N 5/0783; C12N 9/12; C12N 13/00; C12N 15/86

(52) U.S. Cl. CPC A61K 40/11; A61K 40/31; A61K 40/4212; A61K 40/4215; C07K 16/2803; C07K 16/2878; C12N 5/0636; C12N 9/1241; C12N 13/00; C12N 15/86; C07K 2317/31; C07K 2317/622; C12N 2510/00; C12N 2750/14143

(57) ABSTRACT

Compositions and methods for efficient cellular genomic engineering that transduce diverse cell types with minimal toxicity, leading to efficient and stable genomic modifications are described. The compositions and methods are applicable to development of chimeric antigen receptor engineered T cell therapy (CAR-T). An exemplary method introduces a gene of interest into cells by introducing to the cell a viral vector including a transposon encoding the gene of interest and mRNA including a sequence that encodes transposase enzymes configured to mediate targeted integration of the transposon into the cellular genome, whereby the mRNA is introduced to the cell via electroporation. Also disclosed are genetically modified cells and pharmaceutical compositions and methods of use thereof for treating subjects having diseases or disorders.

### CROSS-REFERENCE TO RELATED APPLICATIONS

This application claims the benefit of and priority to U.S. Provisional Application No. 63/319,070 filed on Mar. 11, 2022, the contents of which is incorporated herein in its entirety.

### STATEMENT REGARDING FEDERALLY SPONSORED RESEARCH

This invention was made with Government Support under DP2CA238295, R33CA225498 and R01CA231112 awarded by the National Institutes of Health (NIH), and under W81XWH-20-1-0072 awarded by the US Department of Defense (DoD). The Government has certain rights in the invention.

### REFERENCE TO SEQUENCE LISTING

The Sequence Listing submitted Mar. 11, 2023, as a text file named “YU 8316_PCT_ST26.txt,” created on Mar. 7, 2023, and having a size of 98,304 bytes is hereby incorporated by reference pursuant to 37 C.F.R. § 1.52(e)(5).

### FIELD OF THE INVENTION

The invention is generally related to the fields of gene editing technology and immunotherapy, and more particularly to improved methods of genetic engineering in live cells using mRNA transfection, together with transposon and AAV-mediated targeted gene editing.

### BACKGROUND OF THE INVENTION

Adoptive immunotherapy, in which T cells that are specific for tumor-associated antigens are expanded to generate large numbers of cells and transferred into tumor-bearing hosts, is a promising strategy to treat cancer.

Cellular immunotherapy involves the administration of “living drugs”: genetically modified immune cells that can proliferate, adapt to their environment, engage surrounding cells, and elicit dynamic responses that directly or indirectly target tumor cells for destruction (Hayes, Cellular immunotherapies for cancer. It J Med Sci 190, 41-57, doi:10.1007/s11845-020-02264-w (2021).

Adoptive cell transfer (ACT) is one type of cellular immunotherapy which involves the transfer of cells that directly target tumor cells in the patient (Laskowski & Rezvani, Adoptive cell therapy: Living drugs against cancer. J Exp Med 217, doi:10.1084/jem.20200377 (2020)). One notable ACT approach is chimeric antigen receptor (CAR) T cell therapy, in which T cells are engineered to express a synthetic membrane receptor specific for a tumor antigen. CAR-T therapy has had a remarkable effect in patients with certain hematological malignancies (June, et al. CAR T cell immunotherapy for human cancer. Science 359, 1361-1365, doi:10.1126/science. aar6711 (2018); Majzner, et al. Clinical lessons learned from the first leg of the CAR T cell journey. Nat Med 25, 1341-1355, doi:10.1038/s41591-019-0564-6 (2019)), with five CAR-T products currently approved by the U.S. Food and Drug Administration (FDA) for the treatment of multiple myeloma and B-cell malignancies (Upadhaya, el al. The clinical pipeline for cancer cell therapies. Nat Rev Drug Discov 20, 503-504, doi:10.1038/d41573-021-00100-z (2021)).

The T cells used for adoptive immunotherapy can be generated either by expansion of antigen-specific T cells or redirection of T cells through genetic engineering. One approach to genetically engineering T cells is to modify the cells to target antigens expressed on tumor cells through the expression of chimeric antigen receptors (CARs). CARs are antigen receptors that are designed to recognize cell surface antigens in a human leukocyte antigen-independent manner. Upon recognition and binding of the antigen, the CAR T cell activates an immune response against the antigen bearing cells.

Engineered CAR T cell treatments of patients with cancer have shown promising clinical results. For example, genetically modified T cells expressing anti-CD19 CARs have recently been approved by the FDA for the treatment of patients with relapsed or refractory diffuse large B-cell lymphoma and B-cell acute lymphoblastic leukemia.

However, the majority of current CAR T clinical trials utilize autologous T cells, which are often limited by poor quality and quantity of T cells, as well as the time and expense of manufacturing autologous T cell products. These limitations could be circumvented by the use of allogeneic CAR T cells, further modified to reduce risks of graft-versus-host disease (where the endogenous T cell receptor (TCR) on allogeneic T cells recognize the alloantigens of the recipient) and rejection by the host immune system (e.g., human leukocyte antigen (HLA) on the surface of allogeneic T cells causes rejection by the host). Such modifications encompass both individual and dual disruption of endogenous TCR and HLA class I genes to generate ‘universal’ CAR T cells.

To tackle the myriad challenges that different tumors and tumor environments present, multiple cell-based therapies besides CAR-Ts have been generated, such as tumor infiltrating lymphocytes (TILs) (Verdegaal, et al. Neoantigen landscape dynamics during human melanoma-T cell interactions. Nature 536, 91-95, doi:10.1038/nature18945 (2016)), T cell receptor T (TCR-T) cells (Johnson, et al., Gene therapy with human and mouse T-cell receptors mediates cancer regression and targets normal tissues expressing cognate antigen. Blood 114, 535-546, doi:10.1182/blood-2009-03-211714 (2009); Thomas, et al. NY-ESO-1 Based Immunotherapy of Cancer: Current Perspectives. Front Immunol 9, 947, doi:10.3389/fimmu.2018.00947 (2018)), CAR-NK cells (Chu, et al. CS1-specific chimeric antigen receptor (CAR)-engineered natural killer cells enhance in vitro and in vivo antitumor activity against human multiple myeloma. Leukemia 28, 917-927, doi:10.1038/leu.2013.279 (2014); Genssler, et al. Dual targeting of glioblastoma with chimeric antigen receptor-engineered natural killer cells overcomes heterogeneity of target antigen expression and enhances antitumor activity and survival. Oncoimmunology 5, e1119354, (2016); Zhang, et al. Synergistic Effects of Cabozantinib and EGFR-Specific CAR-NK-92 Cells in Renal Cell Carcinoma. J Immunol Res 2017, 6915912, doi:10.1155/2017/6915912 (2017); Kruschinski, et al. Engineering antigen-specific primary human NK cells against HER-2 positive carcinomas. Proc Natl Acad Sci USA 105, 17481-17486, doi:10.1073/pnas.0804788105 (2008); Liu, et al. Cord blood NK cells engineered to express IL-15 and a CD19-targeted CAR show long-term persistence and potent antitumor activity. Leukemia 32, 520-531, doi:10.1038/leu.2017.226 (2018); Chu, et al. Targeting CD20+ Aggressive B-cell Non-Hodgkin Lymphoma by Anti-CD20 CAR mRNA-Modified Expanded Natural Killer Cells In Vitro and in NSG Mice. Cancer Immunol Res 3, 333-344, doi:10.1158/2326-6066.CIR-14-0114 (2015); and Li, et al., Human iPSC-Derived Natural Killer Cells Engineered with Chimeric Antigen Receptors Enhance Anti-tumor Activity. Cell Stem Cell 23, 181-192 e185, doi:10.1016/j.stem.2018.06.002 (2018)), CAR macrophages (CAR-Ms) (Klichinsky, et al. Human chimeric antigen receptor macrophages for cancer immunotherapy. Nat Biotechnol 38, 947-953, doi:10.1038/s41587-020-0462-y (2020); Zhang, et al. Pluripotent stem cell-derived CAR-macrophage cells with antigen-dependent anti-cancer cell functions. J Hematol Oncol 13, 153, doi:10.1186/s13045-020-00983-2 (2020)), and human induced pluripotent stem cell (iPSC)-derived therapeutic immune cells (Themeli, et al. Generation of tumor-targeted human T lymphocytes from induced pluripotent stem cells for cancer therapy. Nat Biotechnol 31, 928-933, doi:10.1038/nbt.2678 (2013)).

Additional components have been added to CAR constructs to enhance therapeutic efficacy or safety, e.g., kill switch-CARs that can be depleted upon administration of a drug in the case of deleterious CAR toxicity (Di Stasi, et al. Inducible apoptosis as a safety switch for adoptive cell therapy. N Engl J Med 365, 1673-1683, doi:10.1056/NEJMoa1106152 (2011)), or tandem CARs that can target two different antigens (Shah, et al., Bispecific anti-CD20, anti-CD19 CAR T cells for relapsed B cell malignancies: a phase 1 dose escalation and expansion trial. Nat Med 26, 1569-1575, doi:10.1038/s41591-020-1081-3 (2020)). However, a vital part of the implementation of such cellular immunotherapies is the therapeutic cell generation process: whether and how stably engineered therapeutic immune cells can be efficiently generated has critical impact on cell therapy (Morgan & Boyerinas, Genetic Modification of T Cells. Biomedicines 4, doi:10.3390/biomedicines4020009 (2016)).

The majority of current CAR-Ts used in clinical trials were generated using γ-retrovirus or lentivirus for gene transfer. However, several limitations of this family of viral vectors exist. For example, they can be challenging to produce in ultra-high titer (e.g., 1×10¹⁰ μg/mL or higher); their transduction efficiency vary significantly between donors; and their transgene expression may get silenced or dampened (Ellis, Silencing and variegation of gammaretrovirus and lentivirus vectors. Hum Gene Ther 16, 1241-1246, doi:10.1089/hum.2005.16.1241 (2005)). A key limitation of γ-retroviruses specifically is their preference of integration into promoters of active genes (Lukjanov, et al. CAR T-Cell Production Using Nonviral Approaches. J Immunol Res 2021, 6644685, doi:10.1155/2021/6644685 (2021)), including proto-oncogenes as observed in a clinical trial. Genomic studies indeed have found that the insertion profile of γ-retroviruses is associated with higher likelihood of oncogenic transformation (Morgan & Boyerinas, Genetic Modification of T Cells. Biomedicines 4, doi:10.3390/biomedicines4020009 (2016)). HIV-derived lentiviral vectors are considered safer than γ-retroviral vectors based on current gene therapy studies (Kebriaei, et al., Z. Gene Therapy with the Sleeping Beauty Transposon System. Trends Genet 33, 852-870, doi:10.1016/j.tig.2017.08.008 (2017)), but their pathogenic origin and biosafety level 2 or 2+ (BSL2/BSL2+) classification still warrant caution.

Adeno-associated virus (AAV) is commonly used in gene therapy and is considered a safer vector (Naso, et al., Adeno-Associated Virus (AAV) as a Vector for Gene Therapy. BioDrugs 31, 317-334, doi:10.1007/s40259-017-0234-5 (2017)) (consistent with its BSL1 classification). AAV was explored as a candidate for cell therapy vehicle (Bulcha, et al., G. Viral vector platforms within the gene therapy landscape. Signal Transduct Target Ther 6, 53, doi:10.1038/s41392-021-00487-6 (2021)), in part to mitigate the risk of insertional mutagenesis in gene transfer; however, AAV alone is not an ideal CAR carrier—it is non-integrating and CAR expression is thus rapidly diluted during T cell expansion. While AAV can serve as a template for efficient generation of stably integrated CAR-T cells (Eyquem, et al. Targeting a CAR to the TRAC locus with CRISPR/Cas9 enhances tumour rejection. Nature 543, 113-117, doi:10.1038/nature21405 (2017); Dai, et al. One-step generation of modular CAR-T cells with AAV-Cpf1. Nat Methods 16, 247-254, doi:10.1038/s41592-019-0329-7 (2019)), such strategies require genome editing via tools such as CRISPR/Cas; i.e., it requires creation of double-stranded breaks (DSBs) in genomic DNA. It is reported that double stranded breaks (DSBs) and genome editing can trigger the p53 pathway (Enache, et al., Cas9 activates the p53 pathway and selects for p53-inactivating mutations. Nat Genet 52, 662-668, doi:10.1038/s41588-020-0623-4 (2020)), and increase the risk of genomic aberrations such as translocations (e.g., ALPHA2 trial (NCT04416984)).

Alternatively, non-viral systems have been proposed to eliminate the need for viral vectors. However, all current non-viral cell therapy approaches also have their limitations. mRNA electroporation is another CAR-generation strategy, but the duration of CAR expression is extremely short—even for state-of-the-art mRNA technology—due to the instability of mRNA (Morgan, et al., Genetic Modification of T Cells. Biomedicines 4, doi:10.3390/biomedicines4020009 (2016)). Transposon systems such as PiggyBac (Wilson, et al., Jr. PiggyBac transposon-mediated gene transfer in human cells. Mol Ther 15, 139-145, doi:10.1038/sj.mt.6300028 (2007); Doherty, et al. Hyperactive piggyBac gene transfer in human cells and in vivo. Hum Gene Ther 23, 311-320, doi:10.1089/hum.2011.138 (2012)) and Sleeping Beauty (SB) can be used to integrate intact DNA sequences into the genome, avoiding the error-prone reverse transcription step of retroviruses, and transposons are also attractive for their non-pathogenic origin. However, current transposon/transposase approaches for therapeutic cell generation rely predominantly on DNA electroporation or transfection, which is severely limited by inefficient delivery and high cellular toxicity (Kebriaei, et al., Gene Therapy with the Sleeping Beauty Transposon System. Trends Genet 33, 852-870, doi:10.1016/j.tig.2017.08.008 (2017); Monjezi, et al. Enhanced CAR T-cell engineering using non-viral Sleeping Beauty transposition from minicircle vectors. Leukemia 31, 186-194, doi:10.1038/leu.2016.180 (2017)). Non-viral genome editing (e.g., CRISPR) approaches for T cell engineering have been developed (Roth, et al., Reprogramming human T cell function and specificity with non-viral genome targeting. Nature 559, 405-409, doi:10.1038/s41586-018-0326-5 (2018); Roth, et al., Pooled Knockin Targeting for Genome Engineering of Cellular Immunotherapies. Cell 181, 728-744 e721, doi:10.1016/j.cell.2020.03.039 (2020)), however, these also face the two challenges mentioned above: (i) high cellular toxicity associated with electroporation of DNA donor templates and (ii) risk of genome-editing-associated DSBs, genomic aberrations, and p53 pathway.

All current existing cell therapy engineering approaches, both viral and non-viral, have notable limitations. In the context of solid cancers, the efficacy to date of CAR T cell therapy has been variable due to tumor-evolved mechanisms that inhibit local immune cell activity.

Thus, there is an urgent need of alternative approaches for generation of CAR T that simplify the manufacturing process, as well as for improved CAR T therapy that shows reduced risk of immune rejection, reduced exhaustion, and enhanced stability and effector function. Simple and efficient methods are needed for multiplex genomic editing of T cells.

Therefore, it is an object of the invention to provide enhanced methods of gene editing human cells without introducing genomic double-stranded breaks.

It is another object of the invention to provide high efficiency gene editing systems that are highly effective and have low toxicity in a broad range of different cell types.

It is a further object of the invention to provide CAR T cells that exhibit highly stable CAR transgene expression.

It is yet another object of the invention to provide systems for therapeutic cell engineering of various immune cell types, including T cell, natural killer (NK) cell and cells in the myeloid lineage.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### SUMMARY OF THE INVENTION

Compositions and methods for highly efficient cellular genomic engineering (e.g., T cell engineering) that can transduce diverse cell types with minimal cellular toxicity, leading to highly efficient and stable genomic modifications are provided. The disclosed compositions and methods are especially applicable to development of enhanced chimeric antigen receptor engineered T cell therapy (CAR-T).

An exemplary method for introducing a gene of interest into a cell includes introducing to the cell a viral vector including a transposon encoding the gene of interest and mRNA including a sequence that encodes one or more transposase enzymes configured to specifically mediate targeted integration of the transposon into the cellular genome, whereby the mRNA is introduced to the cell via electroporation.

Also disclosed are systems for introducing a gene of interest into a cell, where the system includes a viral vector including a transposon encoding the gene of interest and mRNA including a sequence that encodes one or more transposase enzymes configured to specifically mediate targeted integration of the transposon into the cellular genome.

Also disclosed are kits for introducing a gene of interest into a cell, where the kit includes a viral vector including a transposon encoding the gene of interest and mRNA including a sequence that encodes one or more transposase enzymes configured to specifically mediate targeted integration of the transposon into the cellular genome.

In preferred forms, the transposon is the Sleeping Beauty transposon, and/or the transposase enzyme is the SB100x hyperactive transposase, and/or the viral vector is an Adeno-associated virus (AAV) vector. In a particularly preferred form, the transposon is the Sleeping Beauty transposon, the transposase enzyme is the SB100x hyperactive transposase, and the viral vector is an Adeno-associated virus (AAV) vector.

In some forms, the transposon includes a gene of interest including a reporter gene, a chimeric antigen receptor (CAR), or combinations thereof. In some forms, the transposon further includes a promoter and/or polyadenylation signal operationally linked to the reporter gene and/or the CAR. An exemplary CAR is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof. Exemplary CARs target one or more antigens selected from AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAX5, PD-L1, PDGFR-0, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP-2, Tyrosinase, VEGFR2, WT1, and XAGE.

An exemplary cancer antigen that is recognized by a CAR includes 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-0, TRAIL-R1, TRAIL-R2, tumor antigen CTAA16.88, VEGF-A, VEGFR-1, VEGFR2, and vimentin. In some forms, the CAR is bispecific or multivalent. In a preferred form, the CAR is an anti-CD19 CAR, or an anti-CD22, or an anti-CD19 and anti-CD22 CAR. In a particular form, the CAR is CD19BBz or CD22BBz.

In some forms, the mRNA encoding the transposase includes one or more of N6-methyladenosine (m6A), 5-methylcytosine (m5C), pseudouridine (ψ), N1-methylpseudouridine (me1ψ), 5-methoxyuridine (5moU), a 5′ cap, a poly(A) tail, one or more nuclear localization signals. In some forms, the mRNA, or the transposon, or both are codon optimized for expression in a eukaryotic cell.

In some forms, the mRNA encoding transposase and the viral vector is introduced to the cell at the same or different times. For example, in some forms, the mRNA is introduced to the cell by electroporation at a time point between 10 hours before, and 10 hours after the viral vector including a transposon encoding the gene of interest is introduced to the cell. In some forms, the mRNA is introduced to the cell by electroporation at a time point between one and four hours before the viral vector including a transposon encoding the gene of interest is introduced to the cell. In some forms, the AAV vectors is AAV6 or AAV9.

In exemplary forms, the introduction of mRNA encoding transposase and the viral vector including the transposon is performed ex vivo. In some forms, the cell is a T cell, hematopoietic stem cell (HSC), macrophage, natural killer cell (NK), or dendritic cell (DC). For example, in some forms, the T cell is a CD8+ T cell selected from the group including effector T cells, memory T cells, central memory T cells, and effector memory T cells. In other forms, the T cell is a CD4+ T cell selected from the group including Th1 cells, Th2 cells, Th17 cells, and Treg cells.

Isolated cells modified according to a method for introducing a gene of interest into a cell including introducing to the cell a viral vector including a transposon encoding the gene of interest and mRNA including a sequence that encodes one or more transposase enzymes configured to specifically mediate targeted integration of the transposon into the cellular genome, whereby the mRNA is introduced to the cell via electroporation are also provided. In some forms, the cell includes a gene of interest that is a CAR. In exemplary forms, the CAR is bispecific or multi-specific.

A population of cells derived by expanding cells modified according to a method for introducing a gene of interest into a cell including introducing to the cell a viral vector including a transposon encoding the gene of interest and mRNA including a sequence that encodes one or more transposase enzymes configured to specifically mediate targeted integration of the transposon into the cellular genome, whereby the mRNA is introduced to the cell via electroporation are also provided. Pharmaceutical compositions including the population of cells, together with a pharmaceutically acceptable buffer, carrier, diluent, or excipient are also described.

Methods of treating a subject having a disease, disorder, or condition are also provided. The methods include administering to the subject an effective amount of the pharmaceutical composition including cells modified according to a method for introducing a gene of interest into a cell including introducing to the cell a viral vector including a transposon encoding the gene of interest and mRNA including a sequence that encodes one or more transposase enzymes configured to specifically mediate targeted integration of the transposon into the cellular genome, whereby the mRNA is introduced to the cell via electroporation are also provided. In some forms the methods treat a subject having a disease, disorder, or condition associated with an elevated expression or specific expression of an antigen, whereby the cell includes a CAR that targets the antigen. Methods of treating a subject having a disease, disorder, or condition include administering to the subject an effective amount of a pharmaceutical composition including a genetically modified cell, where the cell is genetically modified by a method including introducing to the cell (i) a viral vector including a transposon encoding the gene of interest; and (ii) mRNA including a sequence that encodes one or more transposase enzymes configured to specifically mediate targeted integration of the transposon into the cellular genome, whereby the mRNA is introduced to the cell via electroporation. In preferred forms, the transposon is the Sleeping Beauty transposon, and/or the transposase enzyme is the SB100x hyperactive transposase, and/or the viral vector is an Adeno-associated virus (AAV) vector. In a particularly preferred form, the transposon is the Sleeping Beauty transposon, the transposase enzyme is the SB100x hyperactive transposase, and the viral vector is an Adeno-associated virus (AAV) vector.

In preferred forms, the transposon encodes one or more gene of interest including a reporter gene, a chimeric antigen receptor (CAR), or combinations thereof. In some forms, the transposon includes a promoter and/or polyadenylation signal operationally linked to the reporter gene and/or the CAR. In exemplary methods, the CAR is specific for an antigen selected from a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof. In another exemplary method, the CAR targets one or more antigens selected from AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAX5, PD-L1, PDGFR-0, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP-2, Tyrosinase, VEGFR2, WT1, and XAGE. Exemplary cancer antigens are selected from 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin. In some forms, the CAR is bispecific or multivalent. In preferred forms, the CAR is anti-CD19 or anti-CD22, or both. In some forms, the CAR is CD19BBz or CD22BBz. In some forms, the AAV vector is AAV6 or AAV9. In preferred forms, the genetically modified cell is a T cell, hematopoietic stem cell (HSC), macrophage, natural killer cell (NK), or dendritic cell (DC). For example, in an exemplary form, the T cell is a CD8+ T cell selected from the group including effector T cells, memory T cells, central memory T cells, and effector memory T cells. In other forms, the T cell is a CD4+ T cell selected from Th1 cells, Th2 cells, Th17 cells, and Treg cells. In exemplary forms, the introduction to the cell is performed ex vivo. For example, in some forms, the cell was isolated from the subject having the disease, disorder, or condition prior to the introduction to the cell. In other forms, the cell was isolated from a healthy donor prior to the introduction to the cell. In preferred forms, the pharmaceutical composition includes a population of cells derived by expanding the genetically modified cell. An exemplary subject is a human subject.

### BRIEF DESCRIPTION OF THE DRAWINGS

The accompanying drawings, which are incorporated in and constitute a part of this specification, illustrate several embodiments of the disclosed method and compositions and together with the description, explain the principles of the disclosed method and compositions.

FIG. 1 is a schematic representation of the MAJESTIC system for generating chimeric antigen receptor (CAR) T cells, showing the two core components of the MAJESTIC system: the Adeno-associated virus (AAV) vector including Sleeping Beauty transposon (SB) carrying desired cell therapy transgenes (AAV-SB-CTx), and the engineered mRNA encoding the hyperactive SB SB100x transposase (SB 100X Transposase mRNA); mRNA electroporation is combined with AAV-delivery of the SB-CT plasmid encoding a CAR to facilitate integration of the SB transposon construct, thus enabling targeted CAR knock-in in human primary cells, such as T cells, NK cells, monocyte cells and macrophages, derived from Induced pluripotent stem cells (iPSC); these cells are then expanded into therapeutic immune cells and introduced to a subject for treatment and prevention of cancer.

FIGS. 2A-2B are graphs showing quantification of CD22.CAR expression via flow cytometry in human CD4 T cells (FIG. 2A) and human CD8 T cells (FIG. 2B), respectively, showing % CD22-CAR (0-60%) for each of AAV-SB-CD22.CAR only (MOI=1E5) (●); mRNA+AAV-SB-CD22.CAR (MOI=1E3) (▪); mRNA+AAV-SB-CD22.CAR (MOI=1E4) (▴); and mRNA+AAV-SB-CD22.CAR (MOI=1E5) (♦), respectively, for each of time points day 3 (d3), d5, d7 and d14, respectively.

FIGS. 3A-3B are graphs showing quantification of BCMA-CAR expression via flow cytometry in human CD4 T cells (FIG. 3A) and human CD8 T cells (FIG. 3B), respectively, showing % CD22-CAR (0-60%) for each of AAV-SB-BCMA.CAR only (MOI=1E5) (●); mRNA+AAV-SB-BCMA.CAR (MOI=1E) (▪); mRNA+AAV-SB-BCMA.CAR (MOI=1E4) (▴); and mRNA+AAV-SB-BCMA.CAR (MOI=1E5) (♦), respectively, for each of time points day 3 (d3), and d5, respectively.

FIGS. 4A-4F are graphs of quantification of the CD22.CAR in human CD4 T cells (FIG. 4A) and human CD8 T cells (FIG. 4B), respectively, for cells transduced with AAV-SB-CD22.CAR virus at various time points, showing % CD22-CAR for each of control samples (no virus or AAV only (iE4), as well as transduction time points (−4 h, −2 h, 1 h, 3 h and 4 h relative to SB100x mRNA electroporation, respectively), for cells at Day 3 (clear) and Day 5 (shaded) post-transduction, respectively. FIGS. 4C-4F are graphs of flow cytometry of CD22.CAR T cells produced via AAV-SB and a titrated serial of SB100X mRNA, with data plotted for % CD22.CAR each of CD8 T cells (FIGS. 4C-4D) and CD4 T cells (FIGS. 4E-4F), respectively, at day 7 (FIGS. 4C, 4E) and at day 12 (FIGS. 4D, 4F), for each of No virus (●), AAV-SB-CD22.CAR only (▪), AAV-SB-CD22.CAR +mRNA, 1 μg (▴), AAV-SB-CD22.CAR+mRNA, 2 μg (▾), AAV-SB-CD22.CAR +mRNA, 4 μg (♦), and AAV-SB-CD22.CAR+mRNA, 8 μg ( [symbol] ), respectively.

FIGS. 5A-5C are graphs of quantification of the CD22.CAR T cells. FIG. 5A shows % 7-AAD negative stained cells (0-100%) after mRNA electroporation, plasmid DNA electroporation, and lentivirus transduction in each of Unstained; No virus (electroporation only); AAV-SB-CD22.CAR only (MOI=1E5); AAV-SB-CD22.CAR +mRNA (MOI=1E5); AAV-SB-CD22.CAR +mRNA (MOI=5E5); Lenti-CD22.CAR (MOI=1); Lenti-CD22.CAR (MOI=10); SB/SB100x plasmid DNA (1 μg); and SB/SB100x plasmid DNA (2 μg) samples, respectively. FIGS. 5B-5C show % CD22.CAR CD8 T Cells at each of day 4 (d4), d7 and d14, respectively (FIG. 5B); and CD22.CAR T Cell number yield (0-3×10⁶) at each of Day 5, Day 9, and Day 14, respectively, for each of No virus (electroporation only) ( [symbol] ); AAV-SB-CD22.CAR only (MOI=1E5) ( [symbol] ); AAV-SB-CD22.CAR +mRNA (MOI=1E5) ( [symbol] ); AAV-SB-CD22.CAR +mRNA (MOI=5E5) ( [symbol] ); Lenti-CD22.CAR (MOI=1) ( [symbol] ); Lenti-CD22.CAR (MOI=10) ( [symbol] ); SB/SB100x plasmid DNA (1 μg) ( [symbol] ); and SB/SB100x plasmid DNA (2 μg) ( [symbol] ) samples, respectively (FIG. 5C).

FIGS. 6A-6D are graphs of Cytolysis (%) of NAML6-GL (NAML6 with GFP and luciferase reporters) cancer cells co-cultured with Lenti-CD22.CAR (clear) or AAV-SB-CD22.CAR (shaded) T cells, respectively (FIGS. 6A-6B), or co-cultured with Lenti-BCMA.CAR (clear) or AAV-SB-BCMA.CAR (shaded) T cells (FIGS. 6C-6D), respectively, seeded at various effector:target (E:T) ratios of 1:2.5, 1:5, 1:10, or 1:20, with luciferase imaging performed at time points 16 h (FIGS. 6A, 6C), or 40 h (FIGS. 6B, 6D), respectively.

FIG. 7A is a schematic representation of how the MAJESTIC system for generating chimeric antigen receptors (CAR) is applied to produce various therapeutic immune cells. The MAJESTIC system includes the steps of isolating T cells from a subject, electroporation with SB100X mRNA and AAV-SB-CAR transduction to produce cells including one or more of single scFv CAR, tandem scFv CAR, TCR-T, and kill-switch CAR cells having an externally-activated ICasp9/Caspase 3-mediated apoptosis “switch”. FIGS. 7B-7C are graphs, showing % cell viability post-electroporation as measured with 7-AAD staining (0-100%) (FIG. 7B), and quantification of HER2.CAR-positive CD8 T cells (0-30%) (FIG. 7C), respectively, for each of No virus (electroporation only); AAV-SB-HER2.CAR only (MOI=1E5); AAV-SB-HER2.CAR+mRNA (MOI=1E5); Lenti-HER2.CAR (MOI=5); and SB/SB 100x plasmid DNA (3 μg) samples, respectively.

FIG. 8A is a schematic representation of an AAV-SB-CD19.20.CAR construct, showing: 5′ITR site, Sleeping Beauty (SB) IR/DR site, EFS, leader sequence, single chain variable domain specific for CD 19 (CD19 scFv) and CD20 scFv CAR sequences joined by a linker to be expressed together as a tandem scFv CAR, CD8 hinge and transmembrane (CD8 TM), 4-1BB, CD3 zeta (CD3z), poly-Adenosine (Poly-A), Sleeping Beauty (SB) IR/DR site and 3′ITR site. FIG. 8B is a graph, showing quantitation of flow cytometry plots to evaluate CAR expression of CD19.20.CAR T cells (CD19.20-CAR (0-40%)) for each, for each of No virus (electroporation only); AAV-SB-CD19.20.CAR only (MOI=1E5); AAV-SB-CD19.20.CAR +mRNA (MOI=1E5); Lenti-CD19.20.CAR (MOI=5); and SB/SB 100x plasmid DNA (3 μg) samples, respectively. FIGS. 8C-8D are graphs of cytolysis analysis of NALM6-GL cancer cells that were co-cultured with lenti-CD19.20.CAR and AAV-SB-CD19.20.CAR T cells, showing cytolysis (%) for each of WT (No virus); AAV-SB-CD19.20.CAR; and lenti-CD19.20.CAR groups, respectively, with E:T ratios of 1:4 or 1:10, in kill assays with leukemia cells after 3 hours (FIG. 8C) or 17 hours (FIG. 8D), respectively.

FIG. 9A is a schematic representation of a construct used to generate conditional control CAR-T cells with two transgenes, CD22 CAR and a suicide-gene (CD22.CAR.iCasp9 T cells), showing: 5′ITR site, Sleeping Beauty (SB) IR/DR site, EFS, leader sequence, single chain variable domain specific for CD 22 (CD22 scFv), CD8 hinge and transmembrane (CD8 TM), 4-1BB, CD3 zeta (CD3z), T2A site, iCasp9 gene, poly-Adenosine (Poly-A), Sleeping Beauty (SB) IR/DR site and 3′ITR site. FIG. 9B is a graph of cell viability post-electroporation as measured with 7-AAD staining, showing 7-AAD negative cells (0-100%) for each of No virus (electroporation only); AAV-SB-CD22.CAR.iCSP9 only (MOI=1E4); AAV-SB-CD22.CAR.iCSP9+mRNA (MOI=1E4); Lenti-CD22.CAR.iCSP9 (MOI=2.5); and SB/SB 100x plasmid DNA (2 μg) samples, respectively. FIG. 9C is a graph of the quantitation of CD22.CAR.iCasp9 T cells post antigen-specific cancer cells stimulation, showing CD22.CAR.iCasp9 T cells (0-60%) for each of No virus; AAV-SB-CD22.CAR.iCSP9+mRNA (MOI=1E4); and Lenti-CD22.CAR.iCSP9 (MOI=2.5) groups, respectively. FIGS. 9D-9E are graphs showing quantification of flow cytometry data of CD22.CAR T cells from human primary CD4 (FIG. 9D) or CD8 (FIG. 9E) T cells, from an independent donor, with CAR-Ts produced by plasmid transposon plasmid, transposon MC, transposon MC with mRNA-transposase, and MAJESTIC (AAV-SB-CD22.CAR+SB100x mRNA). FIG. 9F is a graph of a quantification of time-course flow cytometry data showing % CD22.CAR T cells produced by MAJESTIC and other systems, including no treatment (●); AAV-SB-CD22.CAR only ( [symbol] ); SB/SB 100X plasmid DNA (2 μg+2 μg) (♦); MC-SB/MC-SB 100X plasmid DNA (2 μg+2 μg) (▴); MC-SB (2 μg)+SB100X mRNA (▪); and AAV-SB-CD22.CAR+SB100x mRNA, MOI=1×10⁵ (▾), respectively, generated from PBMCs of four independent healthy human donors (showing mean+/−s.e.m.; f, showing individual donors in separate panels). FIG. 9G is a bar plot of frequencies of insertions in safe harbors, with three biological donors (n=3) mapped for MAJESTIC and MC-SB+SB100X mRNA groups, respectively. Random refers to a set of one million randomly generated genomic coordinates. Insertion coordinates for LV; CD4 (Roth) were obtained from literature (see main for citations). LV=lentivirus, MC=minicircle, SB100X=hyperactive Sleeping Beauty transposase. FIG. 9H is a heatmap of relative quantifications of insertions in functional gene regions for various gene transfer methods. For each condition, the proportion of reads falling into a given gene region is first calculated. Then a fold-change relative to the frequency of randomly generated insertions into that same region produces the value shown in the heatmap. UTR=untranslated regions. The list of cancer genes was taken from COSMIC and included genes from both Tier 1 and Tier 2.

FIGS. 10A-10C are graphs showing CAR-NK, CAR-Monocyte, and CAR-Macrophage generation via MAJESTIC. FIG. 10A shows quantitation of NK92 cells transduced with AAV-SB-HER2.CAR virus (MOI=1E4 and 1E5), transduced with HER2.CAR lentivirus, or electroporated with plasmid DNA (2 μg=1 μg transposon plasmid+1 μg transposase plasmid) at each of time points day 4 (d4), d7 or d14, respectively, for each of No virus (electroporation only); lentiHER2.CAR (MOI=1); lenti-HER2.CAR (MOI=2.5); SB/SB 100x plasmid DNA (2 μg); SB/SB 100x plasmid DNA (4 μg); AAV-SB-HER2.CAR only (MOI=1E5); AAV-SB-HER2.CAR+mRNA (MOI=1E4); AAV-SB-HER2.CAR+mRNA (MOI=1E5), respectively. FIG. 10B is a graph of the quantitation of HER2.CAR T cells (0-50%) for each of No virus (electroporation only); AAV-SB-HER2.CAR+mRNA (MOI=1E4); and AAV-SB-HER2.CAR+mRNA (MOI=1E5) groups, respectively. FIG. 10C is a graph of the quantitation of Cytolysis (0-80%) for each of NK92 and NK92-AAV-SB-HER2.CAR, respectively, at E:T ratios of 1:10 and 1:2 (24 hr), respectively.

FIGS. 11A-11B are graphs showing quantitation of human monocytic cell line THP-1 cells (FIG. 11A) or human CD14+ macrophages (FIG. 11B) transduced with CAR using the MAJESTIC system. FIG. 11A shows % of HER2.CAR (0-100%) in THP-1 cells transduced with No virus, AAV-SB-HER2.CAR virus (MOI=3E5), AAV-SB-HER2.CAR+mRNA (MOI=3E5), or lenti-HER2.CAR (MOI=5), at each of time points day 5 (d5), or day 10 (d10), respectively. FIG. 11B shows % of CD22.CAR (0-100%) in human CD14+ macrophage cells transduced with No virus, AAV-SB-CD22.CAR virus (MOI=5E5), AAV-SB-CD22.CAR+mRNA (MOI=5E5), SB/SB 100x plasmid DNA (3 μg), or lenti-D22.CAR (MOI=10), at each of time points day 5 (d5), or day 11 (d11), respectively.

FIG. 12 is a schematic representation of an exemplary method employing the MAJESTIC system for generating chimeric antigen receptor (CAR)T/NK/macrophage/iPSC generation, showing the preparation of the two core components: the Adeno-associated virus (AAV) vector including Sleeping Beauty transposon carrying desired cell therapy transgenes (AAV-SB-CTx), and the engineered mRNA encoding the hyperactive SB SB100x transposase (SB 100X Transposase mRNA); AAV preparation and expansion; electroporation of CD4 or CD8 T cells with mRNA; AAV-delivery of the SB-CAR plasmid encoding a CAR, PBS washing of transduced and electroporated cells, followed by cell isolation and selection using, e.g., Flow cytometry, and/or cancer cell kill assays.

FIGS. 13A-13D are graphs showing quantitation of cells transduced with CAR using the MAJESTIC system. FIG. 13A shows % of EGFRvIII-specific CAR-T cells (0-20%) transduced with No virus, Lenti-EGFRvIII CAR (MOI=2.5); Lenti-EGFRvIII CAR (MOI=10); AAV-SB-EGFRvIII CAR. only (MOI=1E5), AAV-SB-EGFRvIII CAR.+mRNA (MOI=1E4), and AAV-SB-EGFRvIII CAR.+mRNA (MOI=1E5), respectively. FIG. 13B shows % of NY-ESO-1 TCR-T cells expressing GFP (0-20%) in cells transduced with No virus, AAV-SB-NY-ESO-1.GFP only (MOI=1E5), AAV-SB-NY-ESO-1.GFP+mRNA (MOI=1E5), and SB/SB 100x plasmid DNA (1 g), respectively. FIG. 13C shows % of CD22.CAR. iCASP9 (0-20%) transduced with No virus, AAV-SB-CD22.CAR.iCasp9 only (MOI=1E4), AAV-SB-CD22.CAR.iCasp9+mRNA (MOI=1E4), Lenti-D22.CAR.iCasp9 (MOI=2.5); and SB/SB 100x plasmid DNA (2 μg), respectively. FIG. 13D shows % of HER2.CAR (0-100%) in cells transduced with No virus, AAV-SB-HER2.CAR+mRNA (MOI=2.5E5), SB/SB 100x plasmid DNA (2 μg), or lenti-HER2.CAR (MOI=5), respectively.

FIGS. 14A-14B are graphs of vector copy number (VCN) quantification of MAJESTIC-manufactured CAR-T cells. Purified CAR T cells were collected for DNA extraction after three weeks of mRNA electroporation and viral transduction. Data are for each of four donors, showing VCN (SB left arm)(FIG. 14A), and VCN (SB right arm)(FIG. 14B) for each of no treatment; AAV-SB-CD22.CAR only; MC-SB+SB 100X mRNA; and AAV-SB-CD22.CAR+SB100x mRNA groups, respectively. FIGS. 14C-14D are graphs of SB100X transposase excision efficiency evaluation, showing VCN (four donors; left arm)(FIG. 14C), and VCN (four donors; right arm)(FIG. 14D) for each of no treatment (●); AAV-SB-CD22.CAR only (▴); MC-SB+SB 100X mRNA (▪); and AAV-SB-CD22.CAR+SB100x mRNA (▾) groups, respectively. FIGS. 14E-14F are graphs of SB100X transposase excision efficiency evaluation, showing % excision efficiency for AAV-SB-HER2.CAR+mRNA at days d1-d3 for each of SB left arm (FIG. 14E), and SB right arm (FIG. 14F), respectively.

FIGS. 15A-15G and 15H-15N are graphs of Exhaustion and memory marker expression in CD22-CAR T cells (FIGS. 15A-15G), and in HER2-CAR T cells (FIGS. 15H-15N) before and post transfection for each of PD-1, CTLA4, TIM-3, LAG-3, IL7-Ra, CXCR3 and CXCR7, as indicated.

FIGS. 16A-16B are graphs of survival in NALM6-GL-induced leukemia-bearing NSG mice treated with either PBS (●), untreated CD8 T cells (▪), and AAV-SB-CD22.CAR T cells (▴), respectively, with data plotted as Luminescence (photons/sec) (FIG. 16A), or probability of survival (FIG. 16B) over DPI. Log-rank (Mantel-Cox) tests were performed to evaluate statistical significance.

FIG. 17A is a graphs of Quantification of Flow cytometry data of CD22.CAR T cells from human primary CD3 T cells produced by plasmid transposon plasmid, transposon MC, transposon MC with mRNA-transposase, and MAJESTIC.

FIGS. 17B-17C are graphs showing yield calculations (yield=CAR %*total viable cell count) in total T cells (FIG. 17B) and CD22-CAR T cells (FIG. 17C) at each of days 3, 8 and 14 in AAV-SB-CD22.CAR T cells (●); MC-SB (2 μg)+SB100X mRNA (▪); and AAV-SB-CD22.CAR+mRNA, MOI=1×10⁵ (▴).

FIGS. 18A-18B are graphs of quantitation of the amount of normalized detected chimeric reads, showing numbers of normalized filtered reads per million (RPM) in linear scale (FIG. 18A) or in log 10 scale (FIG. 18B), respectively, for each of AAV-SB transduction and mRNA electroporation (AAV-SB & mRNA) (▴), as compared to the background AAV-SB transduction alone (AAV-SB only) (▪), or PBS treated (●), respectively.

### DETAILED DESCRIPTION OF THE INVENTION

The disclosed method and compositions can be understood more readily by reference to the following detailed description of embodiments and the Examples included therein and to the Figures and their previous and following description.

As it stands, all current existing cell therapy engineering approaches, both viral and non-viral, have notable limitations. In order to provide a system that is capable of generating stably integrated therapeutic immune cells without inducing genomic DSBs, while achieving high efficiency and low cellular toxicity, the advantages of multiple vehicles were combined. As shown in the Examples, the inventors have developed a novel approach to genome modification in general, and CAR T cell development in particular. This system, referred to herein as MAJESTIC (mRNA AAV-Sleeping-Beauty Joint Engineering of Stable Therapeutic Immune Cells), utilizes an AAV vector carrying a Sleeping Beauty (SB) transposon including a construct for introduction of a CAR, as well as electroporation of cells to introduce transposase mRNA. In MAJESTIC, the mRNA component encodes a transposase that mediates a pulse of genomic integration of the Sleeping Beauty (SB) transposon, which carries genes-of-interest and is embedded inside the AAV vector.

This system can transduce diverse immune cell types with minimal cellular toxicity, leading to highly efficient and stable therapeutic cargo delivery. Compared with conventional gene delivery systems, such as lentiviral vector or DNA transposon/transposase electroporation alone, MAJESTIC showed higher cell viability, chimeric antigen receptor (CAR) transgene expression, therapeutic cell yield, as well as prolonged transgene expression. This system also demonstrated versatility for engineering different cell therapy constructs such as canonical CAR, bi-specific CAR, kill switch CAR and synthetic TCR; and for CAR delivery into various immune cells including T cell, natural killer cells, myeloid cells and induced pluripotent stem cells. The targeting CD22-specific CAR-T cells generated by the MAJESTIC system have potency comparable to cells generated by other methods in cancer cell killing.

The MAJESTIC method is simple, which potentiates large-scale manufacturing, and modular, which enables sophisticated genomic (e.g., T-cell) targeting. The MAJESTIC system is readily scalable to high-dimensional CAR-T engineering, such as versatility for engineering different cell therapy constructs such as canonical CAR, bi-specific CAR, kill switch CAR and synthetic T cell receptor (TCR), and for therapeutic cell engineering of various immune cell types including T cell, natural killer (NK) cell and cells in the myeloid lineage (Labanieh, et al., Nature Biomedical Engineering 2:377 (2018)).

While both viral- and non-viral methods for CAR-T engineering and genome editing are viable, the MAJESTIC system combines both. Delivery of the transposase enzyme is mediated by the transient expression following electroporation of cells with transposase mRNA, and delivery of the transposon is mediated by stable AAV transduction. This system therefore reduces the potentially unwanted continuous expression of transposase enzyme, while maintaining the need for stable presence of the CAR.

As demonstrated in the Examples, the simple design of CAR via the MAJESTIC system does not sacrifice other features; rather, it improves CAR stability, transgene expression, effector function, and cancer cell killing ability, while reducing cytotoxicity. The comparative study described in the Examples showed that, when tested in standard laboratory settings, alone and side-by-side with conventional gene delivery systems, such as lentiviral vector or DNA transposon electroporation, measuring transduction efficiency, cell viability, therapeutic cell yield, and stable transgene expression, the MAJESTIC system was much more efficient in generating viable and stable CAR-T cells than lentiviral or DNA transposon electroporation approaches.

### I. Definitions

The term “transposon” or “transposable element” means a nucleic acid sequence, such as a chromosomal segment, that can undergo “transposition”, i.e., to change its position within a genome, especially a segment of DNA encoding one or more genes that can be translocated within a host cell, sometimes creating or reversing mutations and altering the cell's genetic identity and genome size. Exemplary transpositions include introduction of one or more components of plasmid DNA into chromosomal DNA in the absence of a complementary sequence in the host DNA.

The term “transposase” means an enzyme that binds to the end of a transposon and catalyses its movement, e.g., into a genome at a specific point part, by a cut and paste mechanism or a replicative transposition mechanism.

“Introduce” in the context of genome modification refers to bringing in to contact. For example, to introduce a gene editing composition to a cell is to provide contact between the cell and the composition. The term encompasses penetration of the contacted composition to the interior of the cell by any suitable means, e.g., via transfection, electroporation, transduction, gene gun, nanoparticle delivery, etc.

“Homologous” refers to the sequence similarity or sequence identity between two polypeptides or between two nucleic acid molecules. When a position in both of two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or are homologous, then the two sequences are 60% homologous. By way of example, the DNA sequences ATTGCC and TATGGC share 50% homology. Generally, a comparison is made when two sequences are aligned to give maximum homology.

The term “operably linked” or “operationally linked” refers to functional linkage between a regulatory sequence (e.g., promoter, enhancer, silencer, polyadenylation signal, 5′ or 3′ untranslated region (UTR), splice acceptor, IRES, triple helix, 2A self-cleaving peptides such as F2A, E2A, P2A and T2A) and a heterologous nucleic acid sequence permitting them to function in their intended manner (e.g., resulting in expression of the latter). The term encompasses positioning of a regulatory region (sequence), a sequence to be transcribed, and/or a sequence to be translated in a nucleic acid so as to influence transcription or translation of such a sequence. The regulatory sequence can be positioned at any suitable distance from the sequence being regulated (e.g., 1 nucleotide-10,000 nucleotides). For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically includes at least a core (basal) promoter.

“Endogenous” refers to any material from or produced inside an organism, cell, tissue or system. “Exogenous” refers to any material introduced from or produced outside an organism, cell, tissue or system.

The term “antigen” as used herein is defined as a molecule capable of being bound by an antibody or T-cell receptor. An antigen can additionally be capable of provoking an immune response. This immune response can involve either antibody production, or the activation of specific immunologically competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which includes a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the disclosed compositions and methods includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. In the context of cancer, “antigen” refers to an antigenic substance that is produced in a tumor cell, which can therefore trigger an immune response in the host. These cancer antigens can be useful as markers for identifying a tumor cell, which could be a potential candidate/target during treatment or therapy. There are several types of cancer or tumor antigens. There are tumor specific antigens (TSA) which are present only on tumor cells and not on healthy cells, as well as tumor associated antigens (TAA) which are present in tumor cells and on some normal cells. In some forms, the chimeric antigen receptors are specific for tumor specific antigens. In some forms, the chimeric antigen receptors are specific for tumor associated antigens. In some forms, the chimeric antigen receptors are specific both for one or more tumor specific antigens and one or more tumor associated antigens.

“Bi-specific chimeric antigen receptor” refers to a CAR that includes two domains, wherein the first domain is specific for a first ligand/antigen/target, and wherein the second domain is specific for a second ligand/antigen/target. In some forms, the ligand is a B-cell specific protein, a tumor-specific ligand/antigen/target, a tumor associated ligand/antigen/target, or combinations thereof. A bispecific CAR is specific to two different antigens. A multi-specific or multivalent CAR is specific to more than one different antigen, e.g., 2, 3, 4, 5, or more. In some forms, a multi-specific or multivalent CAR targets and/or binds three or more different antigens.

“Encoding” or “encode” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “locus” is the specific physical location of a DNA sequence (e.g., of a gene) on a chromosome. It is understood that a locus of interest can not only qualify a nucleic acid sequence that exists in the main body of genetic material (i.e., in a chromosome) of a cell but also a portion of genetic material that can exist independently to said main body of genetic material such as plasmids, episomes, virus, transposons or in organelles such as mitochondria as non-limiting examples.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. An “isolated nucleic acid” refers to a nucleic acid segment or fragment which has been separated from sequences which flank it in a naturally occurring state, e.g., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

In the context of cells, the term “isolated” also refers to a cell altered or removed from its natural state. That is, the cell is in an environment different from that in which the cell naturally occurs, e.g., separated from its natural milieu such as by concentrating to a concentration at which it is not found in nature. “Isolated cell” is meant to include cells that are within samples that are substantially enriched for the cell of interest and/or in which the cell of interest is partially or substantially purified.

As used herein, “transformed,” “transduced,” and “transfected” encompass the introduction of a nucleic acid or other material into a cell by one of a number of techniques known in the art.

A “vector” is a composition of matter which includes an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” encompasses an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus (AAV) vectors, retroviral vectors, and the like.

“Tumor burden” or “tumor load” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms tumor burden can be assessed using guidelines provided in the Response Evaluation Criteria in Solid Tumors (RECIST).

As used herein, “subject” includes, but is not limited to, animals, plants, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. In some forms, the subject can be any organism in which the disclosed method can be used to genetically modify the organism or cells of the organism.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with or restraining the expression and/or activity of the gene/gene product pathway relative to a standard or a control.

“Treatment” or “treating” means to administer a composition to a subject or a system with an undesired condition (e.g., cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., cancer). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

As used herein, the terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

As used herein, the terms “variant” or “active variant” refers to a polypeptide or polynucleotide that differs from a reference polypeptide or polynucleotide, but retains essential properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Compositions

Compositions for genetic modification of cells are provided. Typically, the compositions include a nucleic acid sequence configured to include a transposon, a viral vector, such as an Adeno-associated virus (AAV) vector, and mRNA encoding a transposase enzyme configured to integrate the transposon into a host cell genome. Typically, the transposon includes nucleic acids encoding one or more genes of interest for insertion at a pre-determined site within a target cell. In some forms, the compositions also include target cells, such as viable cells obtained from a subject, and apparatus for performing genetic manipulation of the target cell using the MAJESTIC system.

In some forms, the compositions are configured to modify the genome of living cells ex vivo when combined according to the disclosed methods for genetic modification of cells. Therefore, in some forms, the compositions are gene editing compositions for use in methods of modifying the genome of a cell. Pharmaceutical compositions containing the modified cells are also provided. As another example, pharmaceutical compositions for use in methods of treating a subject having a disease, disorder, or condition are disclosed. In exemplary forms, the compositions are configured to modify the antigen-recognition function of living immune cells ex vivo (e.g., to produce CAR T cells) for use in methods of treating a subject having a disease, disorder, or condition associated with an elevated expression or specific expression of an antigen. In some exemplary forms, the compositions are configured to produce CAR T cells that selectively target antigen exhibiting an elevated expression or specific expression in a disease, disorder, or condition.

### A. Gene Editing Compositions

Gene editing compositions for use in methods of modifying the genome of a cell are disclosed. Exemplary gene editing compositions for modifying the genome of a cell include a viral vector (e.g., AAV) containing a transposon including a sequence that encodes one or more genes of interest (e.g., a CAR), and mRNA encoding a transposase enzyme capable of directing the insertion of the transposon to one or more chromosomal locations within a target cell.

The viral vector (e.g., AAV) including the transposon and the mRNA encoding the transposase are typically in different compositions and are typically configured to be introduced to the same target cell. For example, a viral vector (e.g., AAV) containing a transposon including a sequence that encodes one or more genes of interest and the mRNA encoding a transposase can be provided in different compositions that are introduced to the cell together or separately. In some forms, after introduction of the mRNA encoding the transposase, the cells can be introduced with the viral vector (e.g., AAV) containing a transposon including a sequence that encodes one or more genes of interest either immediately, or after a certain period of time such as, about 1 h, about 2 h, about 3 h, about 4 h, about 5 h, about 6 h, about 7 h, about 8 h, about 9 h, about 10 h, about 12 h, about 24 h, about 48 h, about 72 h, or about 96 h.

The combination of the compositions including (i) viral vector (e.g., AAV) containing a transposon including a sequence that encodes one or more genes of interest; and (ii) mRNA encoding a transposase enzyme can introduce, induce or otherwise mediate (increase or reduce expression and/or activity) of the one or more genes of interest (e.g., 1, 2, 3, 4, 5, 6, 7, 8, 9, 10, or more genes of interest) within a target cell. For example, in some forms, the combined viral vector (e.g., AAV) containing a transposon including a sequence that encodes one or more exogenous genes (e.g., 1, 2, 3, 4, 5, 6, 7, 8, 9, 10, or more) and mRNA encoding transposase can induce expression of the one or more exogenous genes in a target cell. In other forms, the combined viral vector (e.g., AAV) containing a transposon including a sequence that encodes one or more exogenous genes (e.g., 1, 2, 3, 4, 5, 6, 7, 8, 9, 10, or more) and mRNA encoding transposase causes disruption of one or more chromosomal genes within the target cell, for example, including alterations in the genome (such as, but not limited to, insertions, deletions, translocations, DNA or histone methylation, acetylation, and combinations thereof), resulting in reduced or abolished expression and/or activity of one or more chromosomal gene and/or gene product in the target cell. Methods of determining the expression and/or activity of a gene product are known in the art. These include, but are not limited to, PCR, northern blot, southern blot, western blot, nuclease surveyor assays, sequencing, ELISA, FACS, mRNA-SEQ, single-cell RNA-SEQ, and other molecular biology, chemical, biochemical, cell biology, and immunology assays. A skilled person, based on methods known in the art, and the teachings provided herein would understand how to determine and/or confirm expression and/or alteration of a target gene.

The viral vector (e.g., AAV) containing a transposon including a sequence that encodes one or more genes of interest can be introduced to the target cell through incubation with the cell. The mRNA encoding the transposase enzyme can be introduced to the target cell through non-viral approaches such as physical and/or chemical methods, including via cationic liposomes and polymers, DNA nanoclew, gene gun, microinjection, electroporation, nucleofection, particle bombardment, ultrasound utilization, magnetofection, and conjugation to cell penetrating peptides. Such methods are described for example, in Nayerossadat N., et al., Adv. Biomed. Res., 1:27 (2012) and Lino C A, et al., Drug Deliv., 25(1):1234-1257 (2018). A skilled artisan, based on known delivery methods in the art (e.g., those disclosed in Nayerossadat, et al., and Lino C A., et al) in context of their respective advantages and disadvantages, and the teachings disclosed herein, would be able to determine an optimal method for introduction of the mnRNA encoding transposase enzyme. In a preferred form, mRNA encoding transposase enzyme is introduced to the target cell by electroporation. The electroporation of the target cell can be carried out before or after induction of the same target cell with the viral vector including the transposon. In a preferred form, the viral vector including the transposase enzyme is an Adeno-associated virus (AAV) vector.

### 1. Transposon

The compositions include a nucleic acid Transposon. Transposons translocate from one DNA site to another in a simple, cut-and-paste manner. Transposition is a precise process in which a defined DNA segment is excised from one DNA molecule and moved to another site in the same or different DNA molecule or genome.

Typically, the transposon includes one or more inverted repeats (ITR) that mediate integration into the genome of a host cell, and nucleic acid sequences placed between the ITRs that typically include internal repeats (IR)/direct repeats (DR), a promoter, one or more genes of interest, a Poly A region, and a terminator, internal repeats (IR)/direct repeats (DR).

Translocation of transposon requires specific binding of SB transposase to inverted terminal repeats (ITRs) (e.g., about 230 bp) at each end of the transposon, which is followed by a cut-and-paste transfer of the transposon into a target DNA sequence. The ITRs contain two imperfect direct repeats (DRs) of about 32 bp. The outer DRs are at the extreme ends of the transposon whereas the inner DRs are located inside the transposon, 165-166 bp from the outer DRs. Although there is a core transposase-binding sequence common to all of the DRs, additional adjacent sequences are required for transposition and these sequences vary in the different DRs. In some forms, at least two DRs are required in each ITR for transposition. Each DR appears to have a distinctive role in transposition. Therefore, in some forms, the DRs of a transposase are not interchangeable for efficient transposition. In some forms, the spacing and sequence between the DR elements in an ITR affect transposition rates. In some forms, Transposons are flanked by TA dinucleotide base-pairs that are important for excision. Therefore, in some forms, elimination of the TA motif on one side of the transposon significantly reduces transposition while loss of TAs on both flanks of the transposon abolishes transposition. Exemplary transposons include members of the Tcl/mariner superfamily, such as mariner and Sleeping Beauty (SB) transposons. The regulation, including the strategy to enforce a synapsis of the transposon ends, as well as the requirement for such a synapsis, also varies among recombinases. While mariners have short ITRs with one transposon binding site at each transposon end (Rosenzweig B, et al., 1983. Nucleic Acids Res, 11: 4201-9; Tosi L R and Beverley S M, 2000. Nucleic Acids Res., 28: 784-90.), Sleeping Beauty (SB) belongs to the indirect repeat/direct repeat (IR/DR) subfamily of transposons, possessing two transposase binding sites (represented by direct repeats) at each transposon ends (Franz G and Savakis C, 1991. Nucleic Acids Res, 19: 6646; Izsvak, et al., 1995. Mol Gen Genet. 247: 312-22; Ivics, et al., 1997. Cell, 91: 501-10; Miskey, et al., 2003. Nucleic Acids Res, 31: 6873-81; Plasterk, et al., 1999. Trends Genet, 15: 326-32).

a. Sleeping Beauty Transposon

A preferred transposon is the sleeping beauty (SB) transposon. The Sleeping Beauty transposon system is composed of a Sleeping Beauty (SB) transposase and a transposon that was designed in 1997 to insert specific sequences of DNA into genomes of vertebrate animals.

SB transposition is a cut-and-paste process, during which the transposable element is excised from its original location by the transposase and is integrated into a new location. The transposition process can arbitrarily be divided into at least four major steps: (1) binding of the transposase to its sites within the transposon IRs; (2) formation of a synaptic complex in which the two ends of the elements are paired and held together by transposase subunits; (3) excision from the donor site; and (4) reintegration at a target site.

The natural size of SB is 1.6 kb, and the sequences minimally required for transposition are included in the approximately 230-bp-long IRs. Similar to other transposable elements, the efficiency of SB transposition drops with increasing size, with the upper limit of transposon size being around 10 kb. Therefore, in some forms, the cargo nucleic acid within the SB transposon includes one or more genes of interest having a combined size of 10,000 base pairs (bp), or less than 10,000 bp, such as between about 100 bp and 5,000 bp, inclusive, or between about 500 bp and about 2000 bp.

Different variants of SB transposons are known in the art (see, e.g., WO 98/40510, U.S. Pat. No. 8,227,432, Cui, et al., 2002. Structure-function analysis of the inverted terminal repeats of the Sleeping Beauty transposon”. J. Mol. Biol. 318 (5): 1221-1235; Izsvik, et al. 2000. Sleeping Beauty, a wide host-range transposon vector for genetic transformation in vertebrates. J. Mol. Biol. 302 (1): 93-102). Commercially available plasmids containing Sleeping Beauty transposons are designated
pT (web page addgene.org/26555/sequences/#depositor-partial),
pT2 (web page addgene.org/26557/sequences/#depositor-full) or
pT3 (Yant, et al. Mutational analysis of the N-terminal DNA-binding domain of sleeping beauty transposase: critical residues for DNA binding and hyperactivity in mammalian cells. Mol Cell Biol. 2004 October; 24(20):9239-47.)

In some forms, the transposon is a polynucleotide including a SB transposon including a cargo nucleic acid flanked by left and right inverted terminal repeats (ITR) and left and right inverted repeat/direct repeat (IR/DR) sequences. The left IR contains an additional a motif (HDR) that acts as an enhancer in SB transposition (Izsvak, et al., 2002. J Biol Chem, 277: 34581-8.) The IR/DR is an absolute requirement of SB transposition (Izsvak, et al., 2000. J Mol Biol, 302: 93-102.). Typically, the SB transposon nucleic acid sequence is configured such that the transposon is capable of being mobilized by a Sleeping Beauty transposase protein, i.e., having a left IR/DR including an outer left DR motif and an inner left DR motif, and a right IR/DR including an outer right DR motif and an inner right DR motif, where the IR/DRs each contain binding sites for the SB transposase.

In some forms, the SB IR/DR (Left hand) motif has a nucleic acid sequence of

(SEQ ID NO: 27)
cagttgaagtcggaagtttacatacacttaagttggagtcattaa
aactcgtttttcaactactccacaaatttcttgttaacaaacaat
agttttggcaagtcagttaggacatctactttgtgcatgacacaa
gtcatttttccaacaattgtttacagacagattatttcacttata
attcactgtatcacaattccagtgggtcagaagtttacatacact
aa.

In some forms, the SB IR/DR (Right hand) motif has a nucleic acid sequence of

(SEQ ID NO: 28)
ttgagtgtatgtaaacttctgacccactgggaatgtgatgaaaga
aataaaagctgaaatgaatcattctctctactattattctgatat
ttcacattcttaaaataaagtggtgatcctaactgacctaagaca
gggaatttttactaggattaaatgtcaggaattgtgaaaaagtga
gtttaaatgtatttggctaaggtgtatgtaaacttccgacttcaa
ctg.

In some forms, the SB ITR (Left hand) motif has a nucleic acid sequence of

(SEQ ID NO: 29)
cctgcaggcagctgcgcgctcgctcgctcactgaggccgcccggg
caaagcccgggcgtcgggcgacctttggtcgcccggcctcagtga
gcgagcgagcgcgcagagagggagtggccaactccatcactaggg
gttcct.

In some forms, the SB ITR (Right hand) motif has a nucleic acid sequence of

(SEQ ID NO: 30)
aggaacccctagtgatggagttggccactccctctctgcgcgctc
gctcgctcactgaggccgggcgaccaaaggtcgcccgacgcccgg
gctttgcccgggcggcctcagtgagcgagcgagcgcgcagctgcc
tgcagg.

In some forms, the transposon integrates into the host cell genome at a known or pre-determined location. In some forms, the nucleic acid sequence formed upon integration of the transposon into the genome include any of the following:

(SEQ ID NO: 1)
GATACAGTGCACATGTGGTACAGTTGAAGTCGGAAGTTTA
(SEQ ID NO: 2)
ATCTCAAAATAGTAAATGCTACAGTTGAAGTCGGAAGTTTA
(SEQ ID NO: 3)
AGGTGACTGATACCAAAAATACAGTTGAAGTCGGAAGTTTA
(SEQ ID NO: 4)
ATAGTACAAAGAGTTCTCATATACAGTTGAAGTCGGAAGTTTA
(SEQ ID NO: 5)
AGACAGACCTACAAAGAATATACAGTTGAAGTCGGAAGTTTA
(SEQ ID NO: 6)
GCAAACCAAAATGGCACATGTACAGTTGAAGTCGGAAGTTTA
(SEQ ID NO: 7)
TATTATCAATAGCACCTAATCTACAGTTGAAGTCGGAAGTTTA
(SEQ ID NO: 8)
AAATTTCTAGAAAAGGGTTGGTACAGTTGAAGTCGGAAGTTTA
(SEQ ID NO: 9)
AATGATTATGGCATTCATATATACAGTTGAAGTCGGAAGTTTA
(SEQ ID NO: 10)
CCAGACTTGGTGGCACACACCTACAGTTGAAGTCGGAAGTTTA
(SEQ ID NO: 11)
AAGAGCTTTTATTTACATGAACTACAGTTGAAGTCGGAAGTTTA
(SEQ ID NO: 12)
CGGAACGTGTAGGTTCGTTACATACAGTTGAAGTCGGAAGTTTA
(SEQ ID NO: 13)
AATCCTAGAACTGGAAAATATAGTACAGTTGAAGTCGGAAGTTTA
(SEQ ID NO: 14)
CTGTGAGTGTGGACTGATCAAATATACAGTTGAAGTCGGAAGTTTA
(SEQ ID NO: 15)
ATGACTGTGTCTGCACCTCTATCTACAGTTGAAGTCGGAAGTTTA
(SEQ ID NO: 16)
AGACCCCATATCTCACACCATACATACAGTTGAAGTCGGAAGTTTA
(SEQ ID NO: 17)
AGACCCCATATCTCACACCATACATACAGTTGAAGTCGGAAGTTTA
(SEQ ID NO: 18)
CAAGACCTAGGCCATGCAAGACATACAGTTGAAGTCGGAAGTTTA
(SEQ ID NO: 19)
GGAATCTCTTTTTCTAATTATTGCTACAGTTGAAGTCGGAAGTTTA,

whereby the transposon nucleic acid sequence is indicated in bold font, and the genomic nucleic acid sequence is indicated in non-bold font.
b. Cargo Nucleic Acid

Typically, the transposon includes cargo nucleic acid sequence(s) that is located between the ITR sites in the transposon. The nucleic acid cargo typically includes genes of interest that are to be integrated into the host cell genome. The cargo nucleic acid typically includes one or more of a promoter, one or more genes of interest and one or more terminators.

i. Gene of Interest

In some forms, the nucleic acid cargo includes one or more genes of interest. Typically, the gene(s) of interest include nucleic acid sequences configured to express one or more gene expression products within the host cell. In exemplary forms, a gene of interest is an endogenous or exogenous gene, whose presence or expression within the host cell is desired. In some forms, a gene of interest is a synthetic gene. An exemplary synthetic gene is a gene encoding an engineered polypeptide.

In some forms, the one or more genes of interest within the transposon is codon optimized for expression in a target cell, such as a eukaryotic cell. In some forms, the eukaryotic cell is, or is derived from a particular organism, such as a plant or a mammal, including but not limited to human, or non-human eukaryote or animal or mammal, e.g., mouse, rat, rabbit, dog, livestock, or non-human mammal or primate. Codon-optimization describes gene engineering approaches that use changes of rare codons to synonymous codons that are more frequently used in the cell type of interest with the aim of increasing protein production. In general, codon optimization involves modifying a nucleic acid sequence for enhanced expression in the host cells of interest by replacing at least one codon (e.g., about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Accordingly, in some forms, genes are tailored for optimal gene expression in a given target cell based on codon optimization. Codon usage tables are readily available, for example, at the “Codon Usage Database” available at web page kazusa.orjp/codon/ and these tables can be adapted in a number of ways. See Nakamura, et al., Nucl. Acids Res., 28:292 (2000). Computer algorithms for codon optimizing a particular sequence for expression in a particular host cell are also available, such as Gene Forge (Aptagen; Jacobus, PA), are also available.

In some forms, one or more codons (e.g., 1, 2, 3, 4, 5, 10, 15, 20, 25, 50, or more, or all codons) in a sequence encoding a gene of interest corresponds to the most frequently used codon for a particular amino acid.

An exemplary engineered poly peptide is a chimeric antigen receptor (CAR).

### (a) Chimeric Antigen Receptors (CAR)

In some forms, the gene of interest is a Chimeric Antigen Receptor (CAR).

Immunotherapy using T cells genetically engineered to express a chimeric antigen receptor (CAR) is rapidly emerging as a promising new treatment for haematological and non-haematological malignancies. CARs are engineered receptors that possess both antigen-binding and T-cell-activating functions. Based on the location of the CAR in the membrane of the cell, the CAR can be divided into three main distinct domains, including an extracellular antigen-binding domain, followed by a space region, a transmembrane domain, and the intracellular signaling domain. The antigen-binding domain, most commonly derived from variable regions of immunoglobulins, typically contains VH and VL chains that are joined up by a linker to form the so-called “scFv.” The segment interposing between the antigen-binding domain (e.g., scFv) and the transmembrane domain is a “spacer domain.” The spacer domain can include the constant IgG1 hinge-CH2-CH3 Fc domain. In some cases, the spacer domain and the transmembrane domain are derived from CD8. The intracellular signaling domains mediating T cell activation can include a CD3ζ co-receptor signaling domain derived from C-region of the TCR α and β chains and one or more costimulatory domains.

In some forms, the antigen-binding domain is derived from an antibody. The term antibody herein refers to natural or synthetic polypeptides that bind a target antigen. The term includes polyclonal and monoclonal antibodies, including intact antibodies and functional (e.g., antigen-binding) antibody fragments, including Fab fragments, F(ab′)₂ fragments, Fab′ fragments, Fv fragments, recombinant IgG (rIgG) fragments, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. The term also encompasses intact or full-length antibodies, including antibodies of any class or subclass, including IgG and sub-classes thereof, IgM, IgE, IgA, and IgD. The antigen-binding domain of a CAR can contain complementary determining regions (CDR) of an antibody, variable regions of an antibody, and/or antigen binding fragments thereof. For example, the antigen-binding domain for a CD19 CAR can be derived from a human monoclonal antibody to CD19, such as those described in U.S. Pat. No. 7,109,304, for use in accordance with the disclosed compositions and methods. In some forms, the antigen-binding domain can include an F(ab′)2, Fab′, Fab, Fv or scFv.

In some forms, the CAR includes one or more spacer domain(s) (also referred to as hinge domain) that is located between the extracellular antigen-binding domain and the transmembrane domain. A spacer domain is an amino acid segment that is generally found between two domains of a protein and may allow for flexibility of the protein and movement of one or both of the domains relative to one another. Any amino acid sequence that provides such flexibility and movement of the extracellular antigen-binding domain relative to the transmembrane domain can be used. The spacer domain can be a spacer or hinge domain of a naturally occurring protein. In some forms, the hinge domain is derived from CD8a, such as, a portion of the hinge domain of CD8a, e.g., a fragment containing at least 5 (e.g., 5, 10, 15, 20, 25, 30, 35, or 40) consecutive amino acids of the hinge domain of CD8a. Hinge domains of antibodies, such as an IgG, IgA, IgM, IgE, or IgD antibodies can also be used. In some forms, the hinge domain is the hinge domain that joins the constant CH1 and CH2 domains of an antibody. Non-naturally occurring peptides may also be used as spacer domains. For example, the spacer domain can be a peptide linker, such as a (GxS)n linker, wherein x and n, independently can be an integer of 3 or more, including 3, 4, 5, 6, 7, 8, 9, 10, 11, 12, or more.

In some forms, the CAR includes a transmembrane domain that can be directly or indirectly fused to the antigen-binding domain. The transmembrane domain may be derived either from a natural or a synthetic source. As used herein, a “transmembrane domain” refers to any protein structure that is thermodynamically stable in a cell membrane, preferably a eukaryotic cell membrane. In some forms, the transmembrane domain of the CAR includes a transmembrane domain of an alpha, beta or zeta chain of a T-cell receptor, CD8, CD4, CD28, CD137, CD80, CD86, CD152 or PD1, or a portion thereof. Transmembrane domains can also contain at least a portion of a synthetic, non-naturally occurring protein segment. In some forms, the transmembrane domain is a synthetic, non-naturally occurring alpha helix or beta sheet. In some forms, the protein segment is at least about 15 amino acids, e.g., at least 15, 16, 17, 18, 19, 20, 21, 22, 23, 24, 25, 26, 27, 28, 29, 30, or more amino acids. Examples of synthetic transmembrane domains are known in the art, for example in U.S. Pat. No. 7,052,906 and PCT Publication No. WO 2000/032776.

The intracellular signaling domain is responsible for activation of at least one of the normal effector functions of the immune effector cell expressing the CAR. The term effector function refers to a specialized function of a cell. Effector function of a T cell, for example, may be cytolytic activity or helper activity including the secretion of cytokines. In some forms, an intracellular signaling domain includes the zeta chain of the T cell receptor or any of its homologs (e.g., eta, delta, gamma or epsilon), MB1 chain, B29, Fc RIII, Fc RI and combinations of signaling molecules such as CD3ζ and CD28, 4-1BB, OX40 and combination thereof, as well as other similar molecules and fragments. Intracellular signaling portions of other members of the families of activating proteins can be used, such as FcγRIII and FeεRI.

Many immune effector cells require co-stimulation, in addition to stimulation of an antigen-specific signal, to promote cell proliferation, differentiation and survival, as well as to activate effector functions of the cell. Therefore, in some forms, the CAR includes at least one co-stimulatory signaling domain. The term co-stimulatory signaling domain, refers to at least a portion of a protein that mediates signal transduction within a cell to induce an immune response such as an effector function. The co-stimulatory signaling domain can be a cytoplasmic signaling domain from a co-stimulatory protein, which transduces a signal and modulates responses mediated by immune cells, such as T cells, NK cells, macrophages, neutrophils, or eosinophils. In some forms, the co-stimulatory signaling domain is derived from a co-stimulatory molecule selected from CD27, CD28, CD137, OX40, CD30, CD40, CD3, LFA-1, ICOS, CD2, CD7, LIGHT, NKG2C, B7-H3, ligands of CD83 and combinations thereof.

CARs can be used in order to generate immuno-responsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and PCT Publication WO 9215322). Alternative CAR constructs can be characterized as belonging to successive generations. First-generation CARs typically include a single-chain variable fragment of an antibody specific for an antigen, for example including a VL linked to a VH of a specific antibody, linked by a flexible linker, for example by a CD8α hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, or CD28 signaling domains (for example scFv-CD28-4-1BB-CD3C or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, co-stimulation can be orchestrated by expressing CARs in antigen-specific T cells, chosen so as to be activated and expanded following engagement of their native aβTCR, for example by antigen on professional antigen-presenting cells, with attendant co-stimulation. Any of the first, second, or third generation CARs described above can be used in accordance with the disclosed compositions and methods.

In some forms, the gene of interest within a transposon encodes a CAR targeting one or more antigens specific for cancer, an inflammatory disease, a neuronal disorder, HIV/AIDS, diabetes, a cardiovascular disease, an infectious disease, an autoimmune disease, or combinations thereof. One of skill in the art, based on general knowledge in the field and/or routine experimentation would be able to determine the appropriate antigen to be targeted by a CAR for a specific disease, disorder or condition.

Exemplary antigens specific for cancer that could be targeted by the CAR include, but are not limited to, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, vimentin, and combinations thereof.

In preferred forms, the CAR targets CD19, CD22, or both CD19 and CD22.

Exemplary antigens specific for an inflammatory disease that could be targeted by the CAR include, but are not limited to, AOC3 (VAP-1), CAM-3001, CCL11 (eotaxin-1), CD 125, CD 147 (basigin), CD 154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, Lama glama, LFA-1 (CD 11a), MEDI-528, myostatin, OX-40, rhuMAb β7, scleroscin, SOST, TGF beta 1, TNF-a, VEGF-A, and combinations thereof.

Exemplary antigens specific for a neuronal disorder that could be targeted by the CAR include, but are not limited to, beta amyloid, MABT5102A, and combinations thereof.

Exemplary antigens specific for diabetes that could be targeted by the CAR include, but are not limited to, L-I β, CD3, and combinations thereof.

Exemplary antigens specific for a cardiovascular disease that could be targeted by the CAR include, but are not limited to, C5, cardiac myosin, CD41 (integrin alpha-lib), fibrin II, beta chain, ITGB2 (CD 18), sphingosine-1-phosphate, and combinations thereof.

Exemplary antigens specific for an infectious disease that could be targeted by the CAR include, but are not limited to, anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus, TNF-α, and combinations thereof.

In preferred forms, the CAR targets one or more antigens selected from an antigen listed in Table 1.

TABLE 1
Non-limiting examples of CAR targets

| | | | | | |
|---|---|---|---|---|---|
| AFP | AKAP-4 | ALK | Androgen | B7H3 | BCMA |
| | | | receptor | | |
| Bcr-Abl | BORIS | Carbonic | CD123 | CD138 | CD174 |
| CD19 | CD20 | CD22 | CD30 | CD33 | CD38 |
| CD80 | CD86 | CEA | CEACAM5 | CEACAM6 | Cyclin |
| CYP1B1 | EBV | EGFR | EGFR806 | EGFRvIII | EpCAM |
| EpCAM | EphA2 | ERG | ETV6-AML | FAP | Fos-related |
| | | | | | antigen1 |
| Fucosyl | fusion | GD2 | GD3 | GloboH | GM3 |
| gp100 | GPC3 | HER- | HER2 | HMWMAA | HPV E6/E7 |
| | | 2/neu | | | |
| hTERT | Idiotype | IL12 | IL13RA2 | IM19 | IX |
| LCK | Legumain | lgK | LMP2 | MAD-CT-1 | MAD-CT-2 |
| MAGE | MelanA/MART1 | Mesothelin | MET | ML-IAP | MUC1 |
| Mutant | MYCN | NA17 | NKG2D-L | NY-BR-1 | NY-ESO-1 |
| p53 | | | | | |
| NY-ESO- | OY-TES1 | p53 | Page4 | PAP | PAX3 |
| 1 | | | | | |
| PAX5 | PD-L1 | PDGFR-β | PLAC1 | Polysialic | Proteinase3 |
| | | | | acid | (PR1) |
| PSA | PSCA | PSMA | Ras mutant | RGS5 | RhoC |
| ROR1 | SART3 | sLe(a) | Sperm protein | SSX2 | STn |
| | | | 17 | | |
| Survivin | Tie2 | Tn | TRP-2 | Tyrosinase | VEGFR2 |
| WT1 | XAGE | | | | |

In some forms, the CAR is an anti-CD22 CAR. An exemplary anti-CD22 CAR is CD22BBz, having a nucleic acid sequence:

(SEQ ID NO: 25)
atgcttctgctcgtgacaagcctgctgctgtgcgagctgccccac
cctgcctttctgctgatccctcaggtgcagctgcagcagtctggc
cctggcctcgtgaagcctagccagaccctgagcctgacctgtgcc
atcagcggcgatagcgtgtccagcaatagcgccgcctggaactgg
atcagacagagccctagcagaggcctggaatggctgggccggacc
tactaccggtccaagtggtacaacgactacgccgtgtccgtgaag
tcccggatcaccatcaaccccgacaccagcaagaaccagttctcc
ctgcagctgaacagcgtgacccccgaggataccgccgtgtactac
tgcgccagagaagtgaccggcgacctggaagatgccttcgacatc
tggggccagggcacaatggtcaccgtgtctagcggaggcggcgga
agcgacatccagatgacacagagccccagctccctgagcgccagc
gtgggagacagagtgaccatcacctgtcgggccagccagaccatc
tggtcctacctgaactggtatcagcagcggcctggcaaggccccc
aacctgctgatctatgccgccagctcactgcagagcggcgtgccc
agcagattttccggcagaggcagcggcaccgacttcaccctgaca
atcagttccctgcaggccgaggacttcgccacctactactgccag
cagagctacagcatcccccagaccttcggccaggggaccaagctg
gaaatcaaagcggccgcaggtaccaccacgacgccagcgccgcga
ccaccaacaccggcgcccaccatcgcgtcgcagcccctgtccctg
cgcccagaggcatgccggccagcagcagggggcgcagtgcacacg
agggggctggacttcgcctgtgatatctacatctgggcgcccttg
gccgggacttgtggggtccttctcctgtcactggttatcaccctt
tactgcaaacggggcagaaagaaactcctgtatatattcaaacaa
ccatttatgagaccagtacaaactactcaagaggaagatggctgt
agctgccgatttccagaagaagaagaaggaggatgtgaactgaga
gtgaagttcagcaggagcgcagacgcccccgcgtaccagcagggc
cagaaccagctctataacgagctcaatctaggacgaagagaggag
tacgatgttttggacaagagacgtggccgggaccctgagatgggg
ggaaagccgagaaggaagaaccctcaggaaggcctgtacaatgaa
ctgcagaaagataagatggcggaggcctacagtgagattgggatg
aaaggcgagcgccggaggggcaaggggcacgatggcctttaccag
ggtctcagtacagccaccaaggacacctacgacgcccttcacatg
caggccctgccccctcgc.

The anti-CD22 CAR has an amino acid sequence:

(SEQ ID NO: 26)
MLLLVTSLLLCELPHPAFLLIPQVQLQQSGPGLVKPSQTLSLTCA
ISGDSVSSNSAAWNWIRQSPSRGLEWLGRTYYRSKWYNDYAVSVK
SRITINPDTSKNQFSLQLNSVTPEDTAVYYCAREVTGDLEDAFDI
WGQGTMVTVSSGGGGSDIQMTQSPSSLSASVGDRVTITCRASQTI
WSYLNWYQQRPGKAPNLLIYAASSLQSGVPSRFSGRGSGTDFTLT
ISSLQAEDFATYYCQQSYSIPQTFGQGTKLEIKAAAGTTTTPAPR
PPTPAPTIASQPLSLRPEACRPAAGGAVHTRGLDFACDIYIWAPL
AGTCGVLLLSLVITLYCKRGRKKLLYIFKQPFMRPVQTTQEEDGC
SCRFPEEEEGGCELRVKFSRSADAPAYQQGQNQLYNELNLGRREE
YDVLDKRRGRDPEMGGKPRRKNPQEGLYNELQKDKMAEAYSEIGM
KGERRRGKGHDGLYQGLSTATKDTYDALHMQALPPR.

In other forms, the CAR is an anti-CD19 CAR (e.g., CD19BBz). An exemplary anti-CD19 CAR is CD19BBz.

An exemplary anti CD19 CAR has a nucleic acid sequence:

(SEQ ID NO: 39)
gatatacagatgacacagaccacaagcagcctgagcgcaagcctg
ggcgacagagtgaccatcagctgtagagcctcgcaagacatcagc
aagtacctgaactggtatcagcagaagcccgacggcaccgtgaag
ctgctgatctaccacacaagcagactgcacagcggcgtgcctagc
cggttcagcggcagcggcagcggcaccgattactcgctgaccatc
agtaatctcgagcaagaggacatcgccacctacttctgtcagcaa
ggcaacaccctgccctacacattcggcggcggtaccaagctggaa
ataacgggctcaacaagcggctccggtaagcccggcagtggcgag
ggcagtaccaagggagaggtgaagttacaagagagtggccccggc
ttagtcgccccaagccaaagcctgagcgtgacctgcaccgtgagc
ggcgtgagcctgcccgactacggcgtgagctggatcagacagccc
cctagaaaaggactcgagtggctgggcgtgatctggggcagcgag
accacctactacaacagcgccctgaagagcagactgaccatcatc
aaggacaacagcaagagccaagtgttcctgaagatgaacagcctg
cagaccgacgacaccgccatctactattgtgccaagcactactac
tacggcggcagctacgccatggattattggggccaaggaacctca
gtaacggtcagcagcgcggctgcc.

The anti-CD19 CAR has an amino acid sequence:

(SEQ ID NO: 40)
DIQMTQTTSSLSASLGDRVTISCRASQDISKYLNWYQQKPDGTVK
LLIYHTSRLHSGVPSRFSGSGSGTDYSLTISNLEQEDIATYFCQQ
GNTLPYTFGGGTKLEITGSTSGSGKPGSGEGSTKGEVKLQESGPG
LVAPSQSLSVTCTVSGVSLPDYGVSWIRQPPRKGLEWLGVIWGSE
TTYYNSALKSRLTIIKDNSKSQVFLKMNSLQTDDTAIYYCAKHYY
YGGSYAMDYWGQGTSVTVSSAAA.

An exemplary anti CD20 CAR has a nucleic acid sequence:

(SEQ ID NO: 41)
atgctgctgctggtgacaagcctgctgctgtgcgagctgccccac
cccgccttcctgctgatccccgaggtgcagctgcagcagagcggc
gccgagctggtgaagcccggcgctagcgtgaagatgagctgcaag
gctagcggctacaccttcacaagctataacatgcactgggtgaag
cagacccccggccaaggcctggaatggatcggtgccatctacccc
ggcaacggcgacacaagctataaccaaaaattcaagggcaaggcc
accctgaccgccgacaagagcagcagcaccgcctacatgcagctg
agcagcctgacaagcgaggacagcgccgactactactgcgcaaga
agcaactactacggcagcagctactggttctttgacgtgtggggc
gctggcaccacggtcaccgtatcatctggcgggggcggaagcggc
ggtggaggcagcggcggtggcggttctgacatcgtgctgacacag
agccccgccatcttgagcgctagccccggcgagaaggtgaccatg
acctgcagagctagctcatcggtgaactacatggactggtatcag
aagaaacccggcagcagcccaaagccttggatctacgccacgagc
aatctggcgagcggggtaccccccggttctccggaagcggctcgg
gcacatcatactccctgaccataagccgtgtggaggccgaggacg
ccgccacctactactgtcagcagtggagcttcaaccctccgacct
ttggcggcggcactaaattagagattaag.

The anti CD20 CAR has an amino acid sequence:

(SEQ ID NO: 42)
MLLLVTSLLLCELPHPAFLLIPEVQLQQSGAELVKPGASVKMSCK
ASGYTFTSYNMHWVKQTPGQGLEWIGAIYPGNGDTSYNQKFKGKA
TLTADKSSSTAYMQLSSLTSEDSADYYCARSNYYGSSYWFFDVWG
AGTTVTVSSGGGGSGGGGSGGGGSDIVLTQSPAILSASPGEKVTM
TCRASSSVNYMDWYQKKPGSSPKPWIYATSNLASGVPARFSGSGS
GTSYSLTISRVEAEDAATYYCQQWSFNPPTFGGGTKLEIK.

### Exemplary Bispecific CARs and Construct Design

In some forms, the CAR is a bispecific CAR, i.e., that selectively binds to more than a single antigen. In some forms, the CAR can be multivalent. Bispecific or multi-specific (multivalent) CARs, e.g., including, but not limited to, CARs described in WO 2014/4011988 and US20150038684, are contemplated for use in the disclosed methods and compositions.

In some forms, the CAR is a CD19/CD20 bispecific CAR (CD20.19BBz), having a nucleic acid sequence of

(SEQ ID NO: 31)
atgctgctgctggtgacaagcctgctgctgtgcgagctgccccac
cccgccttcctgctgatccccgaggtgcagctgcagcagagcggc
gccgagctggtgaagcccggcgctagcgtgaagatgagctgcaag
gctagcggctacaccttcacaagctataacatgcactgggtgaag
cagacccccggccaaggcctggaatggatcggtgccatctacccc
ggcaacggcgacacaagctataaccaaaaattcaagggcaaggcc
accctgaccgccgacaagagcagcagcaccgcctacatgcagctg
agcagcctgacaagcgaggacagcgccgactactactgcgcaaga
agcaactactacggcagcagctactggttctttgacgtgtggggc
gctggcaccacggtcaccgtatcatctggcgggggcggaagcggc
ggtggaggcagcggcggtggcggttctgacatcgtgctgacacag
agccccgccatcttgagcgctagccccggcgagaaggtgaccatg
acctgcagagctagctcatcggtgaactacatggactggtatcag
aagaaacccggcagcagcccaaagccttggatctacgccacgagc
aatctggcgagcggggtacccgcccggttctccggaagcggctcg
ggcacatcatactccctgaccataagccgtgtggaggccgaggac
gccgccacctactactgtcagcagtggagcttcaaccctccgacc
tttggcggcggcactaaattagagattaagggcgggggaggtagt
ggcggcgggggcagcggcggtggtgggtccggaggagggggaagc
ggcggcgggggctcagatatacagatgacacagaccacaagcagc
ctgagcgcaagcctgggcgacagagtgaccatcagctgtagagcc
tcgcaagacatcagcaagtacctgaactggtatcagcagaagccc
gacggcaccgtgaagctgctgatctaccacacaagcagactgcac
agcggcgtgcctagccggttcagcggcagcggcagcggcaccgat
tactcgctgaccatcagtaatctcgagcaagaggacatcgccacc
tacttctgtcagcaaggcaacaccctgccctacacattcggcggc
ggtaccaagctggaaataacgggctcaacaagcggctccggtaag
cccggcagtggcgagggcagtaccaagggagaggtgaagttacaa
gagagtggccccggcttagtcgccccaagccaaagcctgagcgtg
acctgcaccgtgagcggcgtgagcctgcccgactacggcgtgagc
tggatcagacagccccctagaaaaggactcgagtggctgggcgtg
atctggggcagcgagaccacctactacaacagcgccctgaagagc
agactgaccatcatcaaggacaacagcaagagccaagtgttcctg
aagatgaacagcctgcagaccgacgacaccgccatctactattgt
gccaagcactactactacggcggcagctacgccatggattattgg
ggccaaggaacctcagtaacggtcagcagcgcggctgcc.

In some forms, the anti-CD20/CD19 bi-specific CAR CD20.19BBz has an amino acid sequence:

(SEQ ID NO: 32)
MLLLVTSLLLCELPHPAFLLIPEVQLQQSGAELVKPGASVKMSCK
ASGYTFTSYNMHWVKQTPGQGLEWIGAIYPGNGDTSYNQKFKGKA
TLTADKSSSTAYMQLSSLTSEDSADYYCARSNYYGSSYWFFDVWG
AGTTVTVSSGGGGSGGGGSGGGGSDIVLTQSPAILSASPGEKVTM
TCRASSSVNYMDWYQKKPGSSPKPWIYATSNLASGVPARFSGSGS
GTSYSLTISRVEAEDAATYYCQQWSFNPPTFGGGTKLEIK.

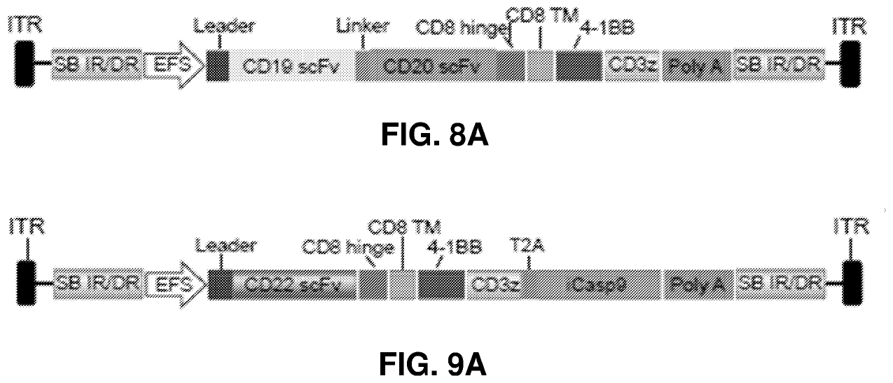

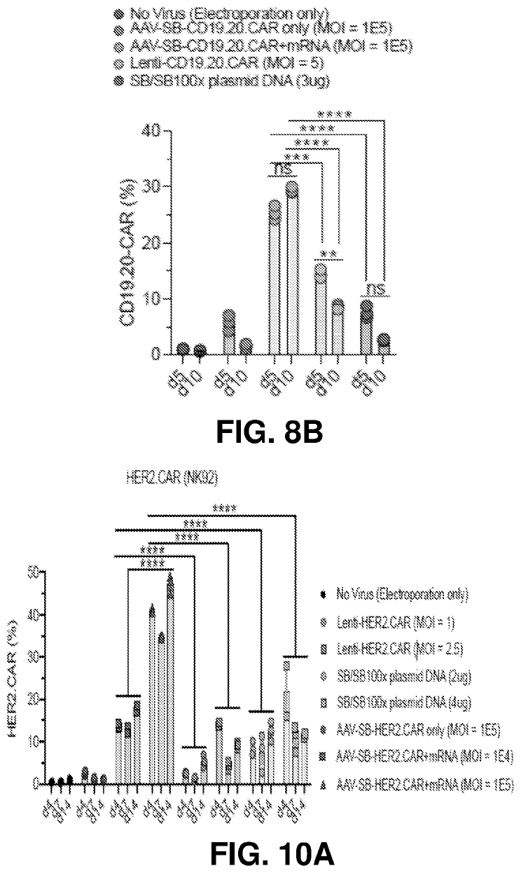

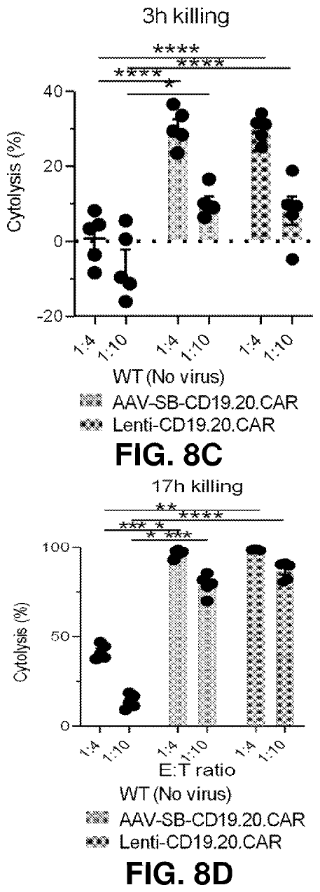

In some forms, the anti-CD20/CD19 bi-specific CAR CD20.19BBz is designed as a construct (AAV-SB-CD19.20.CAR), including the Sleeping Beauty (SB) IR/DR (left) site, EFS promoter, leader sequence, single chain variable domain specific for CD 19 (CD19 scFv) and CD20 scFv CAR sequences joined by a linker to be expressed together as a tandem scFv CAR, CD8 hinge and transmembrane (CD8 TM), 4-1BB, CD3 zeta (CD3z), poly-Adenosine (Poly-A), and Sleeping Beauty (SB) IR/DR (right) site (See, FIG. 8A).

In an exemplary form, an AAV-SB-CD19.20.CAR construct has a nucleic acid sequence:

(SEQ ID NO: 37)
cagttgaagtcggaagtttacatacacttaagttggagtcattaa
aactcgtttttcaactactccacaaatttcttgttaacaaacaat
agttttggcaagtcagttaggacatctactttgtgcatgacacaa
gtcatttttccaacaattgtttacagacagattatttcacttata
attcactgtatcacaattccagtgggtcagaagtttacatacact
aagttgactgtgcctttaaacagcttggaaaattccagaaaatga
tgtcatggctttagagaggatcctaggtcttgaaaggagtgggaa
ttggctccggtgcccgtcagtgggcagagcgcacatcgcccacag
tccccgagaagttggggggaggggtcggcaattgatccggtgcct
agagaaggtggcgcggggtaaactgggaaagtgatgtcgtgtact
ggctccgcctttttcccgagggtgggggagaaccgtatataagtg
cagtagtcgccgtgaacgttctttttcgcaacgggtttgccgcca
gaacacaggaccggttctagacgtacggccaccatgctgctgctg
gtgacaagcctgctgctgtgcgagctgccccaccccgccttcctg
ctgatccccgaggtgcagctgcagcagagcggcgccgagctggtg
aagcccggcgctagcgtgaagatgagctgcaaggctagcggctac
accttcacaagctataacatgcactgggtgaagcagacccccggc
caaggcctggaatggatcggtgccatctaccccggcaacggcgac
acaagctataaccaaaaattcaagggcaaggccaccctgaccgcc
gacaagagcagcagcaccgcctacatgcagctgagcagcctgaca
agcgaggacagcgccgactactactgcgcaagaagcaactactac
ggcagcagctactggttctttgacgtgtggggcgctggcaccacg
gtcaccgtatcatctggcgggggcggaagcggcggtggaggcagc
ggcggtggcggttctgacatcgtgctgacacagagccccgccatc
ttgagcgctagccccggcgagaaggtgaccatgacctgcagagct
agctcatcggtgaactacatggactggtatcagaagaaacccggc
agcagcccaaagccttggatctacgccacgagcaatctggcgagc
ggggtacccgcccggttctccggaagcggctcgggcacatcatac
tccctgaccataagccgtgtggaggccgaggacgccgccacctac
tactgtcagcagtggagcttcaaccctccgacctttggcggcggc
actaaattagagattaagggcgggggaggtagtggcggcgggggc
agcggcggtggtgggtccggaggagggggaagcggcggcgggggc
tcagatatacagatgacacagaccacaagcagcctgagcgcaagc
ctgggcgacagagtgaccatcagctgtagagcctcgcaagacatc
agcaagtacctgaactggtatcagcagaagcccgacggcaccgtg
aagctgctgatctaccacacaagcagactgcacagcggcgtgcct
agccggttcagcggcagcggcagcggcaccgattactcgctgacc
atcagtaatctcgagcaagaggacatcgccacctacttctgtcag
caaggcaacaccctgccctacacattcggcggcggtaccaagctg
gaaataacgggctcaacaagcggctccggtaagcccggcagtggc
gagggcagtaccaagggagaggtgaagttacaagagagtggcccc
ggcttagtcgccccaagccaaagcctgagcgtgacctgcaccgtg
agcggcgtgagcctgcccgactacggcgtgagctggatcagacag
ccccctagaaaaggactcgagtggctgggcgtgatctggggcagc
gagaccacctactacaacagcgccctgaagagcagactgaccatc
atcaaggacaacagcaagagccaagtgttcctgaagatgaacagc
ctgcagaccgacgacaccgccatctactattgtgccaagcactac
tactacggcggcagctacgccatggattattggggccaaggaacc
tcagtaacggtcagcagcgcggctgccaccacgacgccagcgccg
cgaccaccaacaccggcgcccaccatcgcgtcgcagcccctgtcc
ctgcgcccagaggcgtgccggccagcggcggggggcgcagtgcac
acgagggggctggacttcgcctgtgatatctacatctgggcgccc
ttggccgggacttgtggggtccttctcctgtcactggttatcacc
ctttactgccgtttctctgttgttaaacggggcagaaagaaactc
ctgtatatattcaaacaaccatttatgagaccagtacaaactact
caagaggaagatggctgtagctgccgatttccagaagaagaagaa
ggaggatgtgaactgagagtgaagttcagcaggagcgcagacgcc
cccgcgtaccagcagggccagaaccagctctataacgagctcaat
ctaggacgaagagaggagtacgatgttttggacaagagacgtggc
cgggaccctgagatggggggaaagccgagaaggaagaaccctcag
gaaggcctgtacaatgaactgcagaaagataagatggcggaggcc
tacagtgagattgggatgaaaggcgagcgccggaggggcaagggg
cacgatggcctttaccagggtctcagtacagccaccaaggacacc
tacgacgcccttcacatgcaggccctgccccctcgctaggctagc
aataaaagatctttattttcattagatctgtgtgttggttttttg
tgtgaattcttgagtgtatgtaaacttctgacccactgggaatgt
gatgaaagaaataaaagctgaaatgaatcattctctctactatta
ttctgatatttcacattcttaaaataaagtggtgatcctaactga
cctaagacagggaatttttactaggattaaatgtcaggaattgtg
aaaaagtgagtttaaatgtatttggctaaggtgtatgtaaacttc
cgacttcaactg.

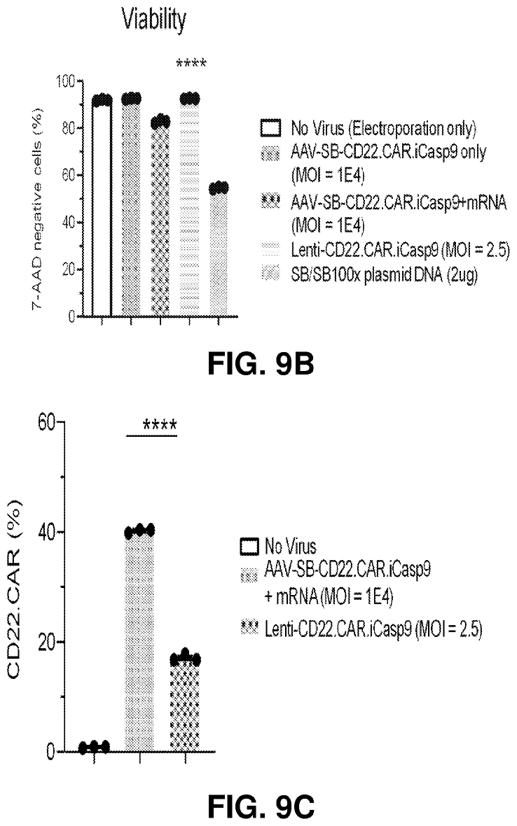

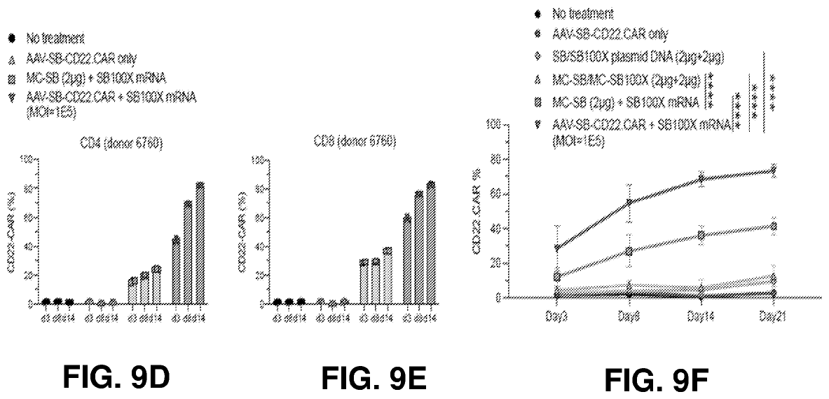

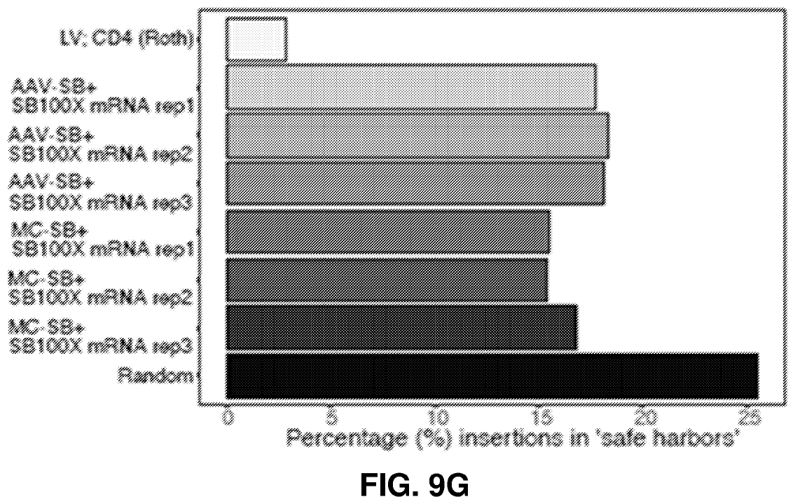

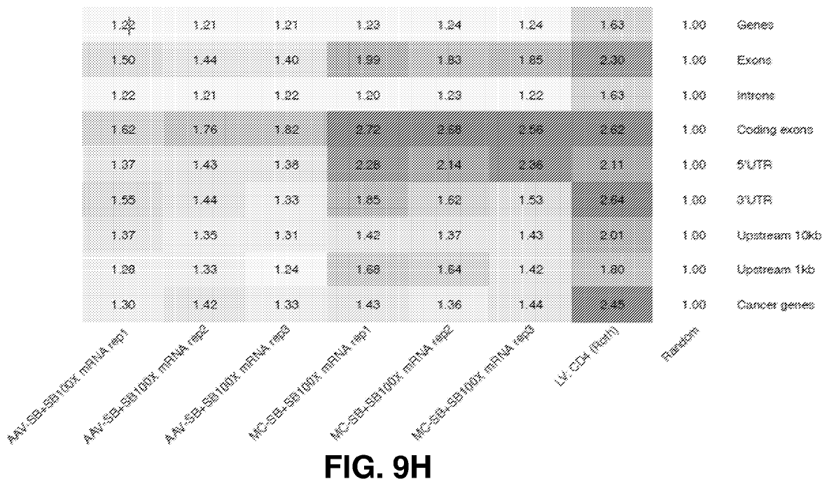

In some forms, a construct is designed to generate conditional control CAR-T cells with two transgenes, e.g., CD22 CAR and a suicide-gene (CD22.CAR.iCasp9). In an exemplary form, a CD22.CAR.iCasp9 construct includes the Sleeping Beauty (SB) IR/DR site (left), EFS promoter, leader sequence, single chain variable domain specific for CD 22 (CD22 scFv), CD8 hinge and transmembrane (CD8 TM), 4-1BB, CD3 zeta (CD3z), T2A site, iCasp9 gene, poly-Adenosine (Poly-A), and the Sleeping Beauty (SB) IR/DR (right) site (See, FIG. 9A).

In an exemplary form, a CD22.CAR.iCasp9 construct has a nucleic acid sequence:

(SEQ ID NO: 38)
cagttgaagtcggaagtttacatacacttaagttggagtcattaa
aactcgtttttcaactactccacaaatttcttgttaacaaacaat
agttttggcaagtcagttaggacatctactttgtgcatgacacaa
gtcatttttccaacaattgtttacagacagattatttcacttata
attcactgtatcacaattccagtgggtcagaagtttacatacact
aagttgactgtgcctttaaacagcttggaaaattccagaaaatga
tgtcatggctttagagaggatcctaggtcttgaaaggagtgggaa
ttggctccggtgcccgtcagtgggcagagcgcacatcgcccacag
tccccgagaagttggggggaggggtcggcaattgatccggtgcct
agagaaggtggcgcggggtaaactgggaaagtgatgtcgtgtact
ggctccgcctttttcccgagggtgggggagaaccgtatataagtg
cagtagtcgccgtgaacgttctttttcgcaacgggtttgccgcca
gaacacaggaccggttctagacgtacggccaccatgcttctgctc
gtgacaagcctgctgctgtgcgagctgccccaccctgcctttctg
ctgatccctcaggtgcagctgcagcagtctggccctggcctcgtg
aagcctagccagaccctgagcctgacctgtgccatcagcggcgat
agcgtgtccagcaatagcgccgcctggaactggatcagacagagc
cctagcagaggcctggaatggctgggccggacctactaccggtcc
aagtggtacaacgactacgccgtgtccgtgaagtcccggatcacc
atcaaccccgacaccagcaagaaccagttctccctgcagctgaac
agcgtgacccccgaggataccgccgtgtactactgcgccagagaa
gtgaccggcgacctggaagatgccttcgacatctggggccagggc
acaatggtcaccgtgtctagcggaggcggcggaagcgacatccag
atgacacagagccccagctccctgagcgccagcgtgggagacaga
gtgaccatcacctgtcgggccagccagaccatctggtcctacctg
aactggtatcagcagcggcctggcaaggcccccaacctgctgatc
tatgccgccagctcactgcagagcggcgtgcccagcagattttcc
ggcagaggcagcggcaccgacttcaccctgacaatcagttccctg
caggccgaggacttcgccacctactactgccagcagagctacagc
atcccccagaccttcggccaggggaccaagctggaaatcaaagcg
gccgcaggtaccaccacgacgccagcgccgcgaccaccaacaccg
gcgcccaccatcgcgtcgcagcccctgtccctgcgcccagaggca
tgccggccagcagcagggggcgcagtgcacacgagggggctggac
ttcgcctgtgatatctacatctgggcgcccttggccgggacttgt
ggggtccttctcctgtcactggttatcaccctttactgcaaacgg
ggcagaaagaaactcctgtatatattcaaacaaccatttatgaga
ccagtacaaactactcaagaggaagatggctgtagctgccgattt
ccagaagaagaagaaggaggatgtgaactgagagtgaagttcagc
aggagcgcagacgcccccgcgtaccagcagggccagaaccagctc
tataacgagctcaatctaggacgaagagaggagtacgatgttttg
gacaagagacgtggccgggaccctgagatggggggaaagccgaga
aggaagaaccctcaggaaggcctgtacaatgaactgcagaaagat
aagatggcggaggcctacagtgagattgggatgaaaggcgagcgc
cggaggggcaaggggcacgatggcctttaccagggtctcagtaca
gccaccaaggacacctacgacgcccttcacatgcaggccctgccc
cctcgcggcagtggagagggcagaggaagtctgctaacatgcggt
gacgtcgaggagaatcctggcccactcgagggagtgcaggtggag
actatctccccaggagacgggcgcaccttccccaagcgcggccag
acctgcgtggtgcactacaccgggatgcttgaagatggaaagaaa
gttgattcctcccgggacagaaacaagccctttaagtttatgcta
ggcaagcaggaggtgatccgaggctgggaagaaggggttgcccag
atgagtgtgggtcagagagccaaactgactatatctccagattat
gcctatggtgccactgggcacccaggcatcatcccaccacatgcc
actctcgtcttcgatgtggagcttctaaaactggaatctggcggt
ggatccggagtcgacggatttggtgatgtcggtgctcttgagagt
ttgaggggaaatgcagatttggcttacatcctgagcatggagccc
tgtggccactgcctcattatcaacaatgtgaacttctgccgtgag
tccgggctccgcacccgcactggctccaacatcgactgtgagaag
ttgcggcgtcgcttctcctcgctgcatttcatggtggaggtgaag
ggcgacctgactgccaagaaaatggtgctggctttgctggagctg
gcgcggcaggaccacggtgctctggactgctgcgtggtggtcatt
ctctctcacggctgtcaggccagccacctgcagttcccaggggct
gtctacggcacagatggatgccctgtgtcggtcgagaagattgtg
aacatcttcaatgggaccagctgccccagcctgggagggaagccc
aagctctttttcatccaggcctgtggtggggagcagaaagaccat
gggtttgaggtggcctccacttcccctgaagacgagtcccctggc
agtaaccccgagccagatgccaccccgttccaggaaggtttgagg
accttcgaccagctggacgccatatctagtttgcccacacccagt
gacatctttgtgtcctactctactttcccaggttttgtttcctgg
agggaccccaagagtggctcctggtacgttgagaccctggacgac
atctttgagcagtgggctcactctgaagacctgcagtccctcctg
cttagggtcgctaatgctgtttcggtgaaagggatttataaacag
atgcctggttgctttaatttcctccggaaaaaacttttctttaaa
acatcagtcgactatccgtacgacgtaccagactacgcactcgac
taagctagcaataaaagatctttattttcattagatctgtgtgtt
ggttttttgtgtgaattcttgagtgtatgtaaacttctgacccac
tgggaatgtgatgaaagaaataaaagctgaaatgaatcattctct
ctactattattctgatatttcacattcttaaaataaagtggtgat
cctaactgacctaagacagggaatttttactaggattaaatgtca
ggaattgtgaaaaagtgagtttaaatgtatttggctaaggtgtat
gtaaacttccgacttcaactg.

### (b) Reporter Genes

In some forms, a gene of interest, includes one or more reporter genes. A reporter gene includes any gene that could be used as an indicator of a successful event, e.g., transfection, transduction, and/or recombination. Reporter genes can allow simple identification and/or measurement of such events. Reporter genes can be fused to regulatory sequences or genes of interest to report expression location or levels, or serve as controls, for example, standardizing transfection efficiencies. Reporter genes include genes that code for fluorescent protein and enzymes that convert invisible substrates to luminescent or colored products.

Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), dTomato, HcRed, DsRed, cyan fluorescent protein (CFP), yellow fluorescent protein (YFP), and autofluorescent proteins including blue fluorescent protein (BFP).

Reporter genes also include selectable markers that confer the ability to grow in the presence of toxic compounds such as antibiotics or herbicides, which would otherwise kill or compromise the cell. A selectable marker can also confer a novel ability to utilize a compound, for example, an unusual carbohydrate or amino acid. Non-limiting examples of selectable markers include genes that confer resistance to Blasticidin, G418/Geneticin, Hygromycin B, Puromycin, or Zeocin.

ii. Promoter

In some forms, the nucleic acid cargo includes one or more promoter elements, configured to control expression of the one or more genes of interest upon integration within the host cell genome.

In some forms, the nucleic acid cargo includes genes that are to be kept uncontrol of an endogenous promoter (e.g., a promoter at or near the site of integration). For example, the transgene can contain a splice acceptor/donor, 2A peptide, and/or internal ribosome entry site (IRES) operationally linked to a gene of interest (e.g., reporter gene, CAR) to allow expression of the transgene in frame with a gene at the site of integration and/or under the control of the promoter at the site of integration.

In other forms, it is desired that the transgene be under the control of an exogenous promoter, such as a constitutive promoter or an inducible promoter. In such cases, the transgene includes a promoter (e.g., EFS or tetracycline-inducible promoter) operationally linked to a gene of interest (e.g., reporter gene, CAR). In some forms, the transposon does not contain a promoter operationally linked to the transgene (e.g., reporter gene, CAR). In some forms, the promoter is a strong, constitutively active promoter for high-level expression of the gene of interest. Commonly used promoters of this type include the CMV (cytomegalovirus) promoter/enhancer, EF1a (elongation factor 1a), SV40 (simian virus 40), chicken β-actin and CAG (CMV, chicken β-actin, rabbit β-globin). A preferred promoter is the EF1 alpha, or EFS (its short, intron-less form) promoter. EFS is a cellular-derived enhancer/promoter with decreased cross-activation of nearby promoters, therefore hypothetically decreasing the risk of genotoxicity. In the design and construction of viral vectors, multiple transcription units may be arranged in close proximity in a space-limited vector. All of these promoters provide constitutively active, high-level gene expression in most cell types. Some of these promoters are subject to silencing in certain cell types, therefore this consideration should be evaluated for each application.

2. Transposase mRNA

Provided as part of the gene editing compositions are one or more mRNAs that encode transposase enzymes. Compositions of transposase enzyme mRNA that are provided in combination with a transposon are configured to perform transposition of the specific transposase.

The mRNA encoding transposase enzyme can be modified or unmodified. In some forms, the mRNA is modified to reduce immunogenicity, to optimize translation, and/or to confer increased stability and/or expression of the transposase enzyme. The modified mRNA can incorporate several chemical changes to the nucleotides, including changes to the nucleobase, the ribose sugar, and/or the phosphodiester linkage. These modified mRNAs can improve efficiency of the transposase enzyme (i.e., increase transposase enzyme protein levels), reduce cellular toxicity, and/or increase mRNA stability relative to the unmodified mRNA. (Li, et al., Nat. Biomed. Eng., 1(5): pii: 0066 (2017) and WO 2017/181107 disclose compositions and methods of modifying mRNAs).

In some forms, the mRNA encoding transposase enzyme contains modifications such as N6-methyladenosine (m6A), 5-methylcytosine (m5C), pseudouridine (ψ), N1-methylpseudouridine (me1ψ), and 5-methoxyuridine (5moU); a 5′ cap; a poly(A) tail; one or more nuclear localization signals; or combinations thereof.

In some forms, the mRNA encoding transposase enzyme is codon optimized for expression in a target cell, such as a eukaryotic cell. In some forms, the eukaryotic cell is, or is derived from a particular organism, such as a plant or a mammal, including but not limited to human, or non-human eukaryote or animal or mammal, e.g., mouse, rat, rabbit, dog, livestock, or non-human mammal or primate. Codon-optimization describes gene engineering approaches that use changes of rare codons to synonymous codons that are more frequently used in the cell type of interest with the aim of increasing protein production. In general, codon optimization involves modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, in some forms, genes are tailored for optimal gene expression in a given organism based on codon optimization. Codon usage tables are readily available, for example, at the “Codon Usage Database” available at web page kazusa.orjp/codon/and these tables can be adapted in a number of ways. See Nakamura, et al., Nucl. Acids Res., 28:292 (2000). Computer algorithms for codon optimizing a particular sequence for expression in a particular host cell are also available, such as Gene Forge (Aptagen; Jacobus, PA), are also available.

a. Sleeping Beauty Transposase

In preferred forms, the transposase is a Sleeping Beauty (SB) transposase, configured to transpose the Sleeping Beauty (SB) transposon within a target cell. The transposase binding sites of SB elements are repeated twice per IR in a direct orientation (DRs). This special organization of inverted repeat is termed IR/DR and is a strict requirement for transposition. Specific binding of SB transposase to the DRs is mediated by an N-terminal, paired-like DNA-binding domain of the transposase that overlaps with a nuclear localization signal. An AT-hook motif has also been identified as a functional subdomain contributing to specific DNA binding. The catalytic domain of the SB transposase mediates the DNA breakage and integration reactions and is characterized by the DDE signature, an evolutionarily conserved domain also found in some bacterial transposases, retrotransposon/retrovirus integrases, and the RAG1 immunoglobulin gene recombinase. The catalytic domains of DDE recombinases have been shown to be also involved in mediating interactions with the target DNA.

i. Hyperactive SB Transposase

In preferred forms, the SB transposase is a hyperactive SB transposase. Hyperactive transposases have been derived from the original SB transposase by mutagenesis of the catalytic and DNA-binding domains.

In an exemplary form, the hyperactive SB transposase is the SB100X transposase. The SB100X transposase, engineered using a combination of in vitro molecular evolution and selection, is the most active of the transposases generated so far, and was named molecule of the year for 2009 (internet page iscspm.webnode.com/). This powerful transposase directs the highest levels of transposon integration yet demonstrated, presumably due to increased stability (Mit6s, et al., Molecular evolution of a novel hyperactive Sleeping Beauty transposase enables robust stable gene transfer in vertebrates. Nat. Genet. 2009; 41:753-761; Grabundzija, et al., Comparative analysis of transposable element vector systems in human cells. Mol. Ther. 2010; 18:1200-1209).

In some forms, the transposase is the SB100X transposase having a DNA sequence:

(SEQ ID NO: 33)
atgggaaaatcaaaagaaatcagccaagacctcagaaaaagaatt
gtagacctccacaagtctggttcatccttgggagcaatttccaaa
cgcctggcggtaccacgttcatctgtacaaacaatagtacgcaag
tataaacaccatgggaccacgcagccgtcataccgctcaggaagg
agacgcgttctgtctcctagagatgaacgtactttggtgcgaaaa
gtgcaaatcaatcccagaacaacagcaaaggaccttgtgaagatg
ctggaggaaacaggtacaaaagtatctatatccacagtaaaacga
gtcctatatcgacataacctgaaaggccactcagcaaggaagaag
ccactgctccaaaaccgacataagaaagccagactacggtttgca
actgcacatggggacaaagatcgtactttttggagaaatgtcctc
tggtctgatgaaacaaaaatagaactgtttggccataatgaccat
cgttatgtttggaggaagaagggggaggcttgcaagccgaagaac
accatcccaaccgtgaagcacgggggtggcagcatcatgttgtgg
gggtgctttgctgcaggagggactggtgcacttcacaaaatagat
ggcatcatggacgcggtgcagtatgtggatatattgaagcaacat
ctcaagacatcagtcaggaagttaaagcttggtcgcaaatgggtt
ttccaacacgacaatgaccccaagcatacttccaaagttgtggca
aaatggcttaaggacaacaaagtcaaggtattggagtggccatca
caaagccctgacctcaatcctatagaaaatttgtgggcagaactg
aaaaagcgtgtgcgagcaaggaggcctacaaacctgactcagtta
caccagctctgtcaggaggaatgggccaaaattcacccaaattat
tgtgggaagcttgtggaaggctacccgaaacgtttgacccaagtt
aaacaatttaaaggcaatgctaccaaatactag.

In some forms, the transposase is the SB100X transposase having RNA sequence

(SEQ ID NO: 34)
augggaaaaucaaaagaaaucagccaagaccucagaaaaagaauu
guagaccuccacaagucugguucauccuugggagcaauuuccaaa
cgccuggcgguaccacguucaucuguacaaacaauaguacgcaag
uauaaacaccaugggaccacgcagccgucauaccgcucaggaagg
agacgcguucugucuccuagagaugaacquacuuuggugcgaaaa
gugcaaaucaaucccagaacaacagcaaaggaccuugugaagaug
cuggaggaaacagguacaaaaguaucuauauccacaguaaaacga
guccuauaucgacauaaccugaaaggccacucagcaaggaagaag
ccacugcuccaaaaccgacauaagaaagccagacuacgguuugca
acugcacauggggacaaagaucguacuuuuuggagaaauguccuc
uggucugaugaaacaaaaauagaacuguuuggccauaaugaccau
cguuauguuuggaggaagaagggggaggcuugcaagccgaagaac
accaucccaaccgugaagcacggggguggcagcaucauguugugg
gggugcuuugcugcaggagggacuggugcacuucacaaaauagau
ggcaucauggacgcggugcaguauguggauauauugaagcaacau
cucaagacaucagucaggaaguuaaagcuuggucgcaaauggguu
uuccaacacgacaaugaccccaagcauacuuccaaaguuguggca
aaauggcuuaaggacaacaaagucaagguauuggaguggccauca
caaagcccugaccucaauccuauagaaaauuugugggcagaacug
aaaaagcgugugcgagcaaggaggccuacaaaccugacucaguua
caccagcucugucaggaggaaugggccaaaauucacccaaauuau
ugugggaagcuuguggaaggcuacccgaaacguuugacccaaguu
aaacaauuuaaaggcaaugcuaccaaauacuag.

In some forms, the SB100X transposase has amino acid sequence

(SEQ ID NO: 35)
MGKSKEISQDLRKRIVDLHKSGSSLGAISKRLAVPRSSVQTIVRK
YKHHGTTQPSYRSGRRRVLSPRDERTLVRKVQINPRITAKDLVKM
LEETGTKVSISTVKRVLYRHNLKGHSARKKPLLQNRHKKARLRFA
TAHGDKDRTFWRNVLWSDETKIELFGHNDHRYVWRKKGEACKPKN
TIPTVKHGGGSIMLWGCFAAGGTGALHKIDGIMDAVQYVDILKQH
LKTSVRKLKLGRKWVFQHDNDPKHISKVVAKWLKDNKVKVLEWPS
QSPDLNPIENLWAELKKRVRARRPTNLTQLHQLCQEEWAKIHPNY
CGKLVEGYPKRLTQVKQFKGNATKY.

In some forms, transposase enzyme mRNA is prepared by in vitro transcription, for example, from a plasmid including the transposase nucleic acid sequence. Exemplary plasmids including the SB100X nucleic acid sequence include pcDNA3.1, having a nucleic acid sequence

(SEQ ID NO: 36)
gacggatcgggagatctcccgatcccctatggtgcactctcagta
caatctgctctgatgccgcatagttaagccagtatctgctccctg
cttgtgtgttggaggtcgctgagtagtgcgcgagcaaaatttaag
ctacaacaaggcaaggcttgaccgacaattgcatgaagaatctgc
ttagggttaggcgttttgcgctgcttcgcgatgtacgggccagat
atacgcgttgacattgattattgactagttattaatagtaatcaa
ttacggggtcattagttcatagcccatatatggagttccgcgtta
cataacttacggtaaatggcccgcctggctgaccgcccaacgacc
cccgcccattgacgtcaataatgacgtatgttcccatagtaacgc
caatagggactttccattgacgtcaatgggtggagtatttacggt
aaactgcccacttggcagtacatcaagtgtatcatatgccaagta
cgccccctattgacgtcaatgacggtaaatggcccgcctggcatt
atgcccagtacatgaccttatgggactttcctacttggcagtaca
tctacgtattagtcatcgctattaccatggtgatgcggttttggc
agtacatcaatgggcgtggatagcggtttgactcacggggatttc
caagtctccaccccattgacgtcaatgggagtttgttttggcacc
aaaatcaacgggactttccaaaatgtcgtaacaactccgccccat
tgacgcaaatgggcggtaggcgtgtacggtgggaggtctatataa
gcagagctctctggctaactagagaacccactgcttactggctta
tcgaaattaatacgactcactatagggagacccaagctggctagc
caccatgggaaaatcaaaagaaatcagccaagacctcagaaaaag
aattgtagacctccacaagtctggttcatccttgggagcaatttc
caaacgcctggcggtaccacgttcatctgtacaaacaatagtacg
caagtataaacaccatgggaccacgcagccgtcataccgctcagg
aaggagacgcgttctgtctcctagagatgaacgtactttggtgcg
aaaagtgcaaatcaatcccagaacaacagcaaaggaccttgtgaa
gatgctggaggaaacaggtacaaaagtatctatatccacagtaaa
acgagtcctatatcgacataacctgaaaggccactcagcaaggaa
gaagccactgctccaaaaccgacataagaaagccagactacggtt
tgcaactgcacatggggacaaagatcgtactttttggagaaatgt
cctctggtctgatgaaacaaaaatagaactgtttggccataatga
ccatcgttatgtttggaggaagaagggggaggcttgcaagccgaa
gaacaccatcccaaccgtgaagcacgggggtggcagcatcatgtt
gtgggggtgctttgctgcaggagggactggtgcacttcacaaaat
agatggcatcatggacgcggtgcagtatgtggatatattgaagca
acatctcaagacatcagtcaggaagttaaagcttggtcgcaaatg
ggttttccaacacgacaatgaccccaagcatacttccaaagttgt
ggcaaaatggcttaaggacaacaaagtcaaggtattggagtggcc
atcacaaagccctgacctcaatcctatagaaaatttgtgggcaga
actgaaaaagcgtgtgcgagcaaggaggcctacaaacctgactca
gttacaccagctctgtcaggaggaatgggccaaaattcacccaaa
ttattgtgggaagcttgtggaaggctacccgaaacgtttgaccca
agttaaacaatttaaaggcaatgctaccaaatactagaagcttaa
gtttaaaccgctgatcagcctcgactgtgccttctagttgccagc
catctgttgtttgcccctcccccgtgccttccttgaccctggaag
gtgccactcccactgtcctttcctaataaaatgaggaaattgcat
cgcattgtctgagtaggtgtcattctattctggggggtggggtgg
ggcaggacagcaagggggaggattgggaagacaatagcaggcatg
ctggggatgcggtgggctctatggcttctgaggcggaaagaacca
gctggggctctagggggtatccccacgcgccctgtagcggcgcat
taagcgcggcgggtgtggtggttacgcgcagcgtgaccgctacac
ttgccagcgccctagcgcccgctcctttcgctttcttcccttcct
ttctcgccacgttcgccggctttccccgtcaagctctaaatcggg
ggctccctttagggttccgatttagtgctttacggcacctcgacc
ccaaaaaacttgattagggtgatggttcacgtagtgggccatcgc
cctgatagacggtttttcgccctttgacgttggagtccacgttct
ttaatagtggactcttgttccaaactggaacaacactcaacccta
tctcggtctattcttttgatttataagggattttgccgatttcgg
cctattggttaaaaaatgagctgatttaacaaaaatttaacgcga
attaattctgtggaatgtgtgtcagttagggtgtggaaagtcccc
aggctccccagcaggcagaagtatgcaaagcatgcatctcaatta
gtcagcaaccaggtgtggaaagtccccaggctccccagcaggcag
aagtatgcaaagcatgcatctcaattagtcagcaaccatagtccc
gcccctaactccgcccatcccgcccctaactccgcccagttccgc
ccattctccgccccatggctgactaattttttttatttatgcaga
ggccgaggccgcctctgcctctgagctattccagaagtagtgagg
aggcttttttggaggcctaggcttttgcaaaaagctcccgggagc
ttgtatatccattttcggatctgatcaagagacaggatgaggatc
gtttcgcatgattgaacaagatggattgcacgcaggttctccggc
cgcttgggtggagaggctattcggctatgactgggcacaacagac
aatcggctgctctgatgccgccgtgttccggctgtcagcgcaggg
gcgcccggttctttttgtcaagaccgacctgtccggtgccctgaa
tgaactgcaggacgaggcagcgcggctatcgtggctggccacgac
gggcgttccttgcgcagctgtgctcgacgttgtcactgaagcggg
aagggactggctgctattgggcgaagtgccggggcaggatctcct
gtcatctcaccttgctcctgccgagaaagtatccatcatggctga
tgcaatgcggcggctgcatacgcttgatccggctacctgcccatt
cgaccaccaagcgaaacatcgcatcgagcgagcacgtactcggat
ggaagccggtcttgtcgatcaggatgatctggacgaagagcatca
ggggctcgcgccagccgaactgttcgccaggctcaaggcgcgcat
gcccgacggcgaggatctcgtcgtgacccatggcgatgcctgctt
gccgaatatcatggtggaaaatggccgcttttctggattcatcga
ctgtggccggctgggtgtggcggaccgctatcaggacatagcgtt
ggctacccgtgatattgctgaagagcttggcggcgaatgggctga
ccgcttcctcgtgctttacggtatcgccgctcccgattcgcagcg
catcgccttctatcgccttcttgacgagttcttctgagcgggact
ctggggttcgaaatgaccgaccaagcgacgcccaacctgccatca
cgagatttcgattccaccgccgccttctatgaaaggttgggcttc
ggaatcgttttccgggacgccggctggatgatcctccagcgcggg
gatctcatgctggagttcttcgcccaccccaacttgtttattgca
gcttataatggttacaaataaagcaatagcatcacaaatttcaca
aataaagcatttttttcactgcattctagttgtggtttgtccaaa
ctcatcaatgtatcttatcatgtctgtataccgtcgacctctagc
tagagcttggcgtaatcatggtcatagctgtttcctgtgtgaaat
tgttatccgctcacaattccacacaacatacgagccggaagcata
aagtgtaaagcctggggtgcctaatgagtgagctaactcacatta
attgcgttgcgctcactgcccgctttccagtcgggaaacctgtcg
tgccagctgcattaatgaatcggccaacgcgcggggagaggcggt
ttgcgtattgggcgctcttccgcttcctcgctcactgactcgctg
cgctcggtcgttcggctgcggcgagcggtatcagctcactcaaag
gcggtaatacggttatccacagaatcaggggataacgcaggaaag
aacatgtgagcaaaaggccagcaaaaggccaggaaccgtaaaaag
gccgcgttgctggcgtttttccataggctccgcccccctgacgag
catcacaaaaatcgacgctcaagtcagaggtggcgaaacccgaca
ggactataaagataccaggcgtttccccctggaagctccctcgtg
cgctctcctgttccgaccctgccgcttaccggatacctgtccgcc
tttctcccttcgggaagcgtggcgctttctcatagctcacgctgt
aggtatctcagttcggtgtaggtcgttcgctccaagctgggctgt
gtgcacgaaccccccgttcagcccgaccgctgcgccttatccggt
aactatcgtcttgagtccaacccggtaagacacgacttatcgcca
ctggcagcagccactggtaacaggattagcagagcgaggtatgta
ggcggtgctacagagttcttgaagtggtggcctaactacggctac
actagaagaacagtatttggtatctgcgctctgctgaagccagtt
accttcggaaaaagagttggtagctcttgatccggcaaacaaacc
accgctggtagcggtggtttttttgtttgcaagcagcagattacg
cgcagaaaaaaaggatctcaagaagatcctttgatcttttctacg
gggtctgacgctcagtggaacgaaaactcacgttaagggattttg
gtcatgagattatcaaaaaggatcttcacctagatccttttaaat
taaaaatgaagttttaaatcaatctaaagtatatatgagtaaact
tggtctgacagttaccaatgcttaatcagtgaggcacctatctca
gcgatctgtctatttcgttcatccatagttgcctgactccccgtc
gtgtagataactacgatacgggagggcttaccatctggccccagt
gctgcaatgataccgcgagacccacgctcaccggctccagattta
tcagcaataaaccagccagccggaagggccgagcgcagaagtggt
cctgcaactttatccgcctccatccagtctattaattgttgccgg
gaagctagagtaagtagttcgccagttaatagtttgcgcaacgtt
gttgccattgctacaggcatcgtggtgtcacgctcgtcgtttggt
atggcttcattcagctccggttcccaacgatcaaggcgagttaca
tgatcccccatgttgtgcaaaaaagcggttagctccttcggtcct
ccgatcgttgtcagaagtaagttggccgcagtgttatcactcatg
gttatggcagcactgcataattctcttactgtcatgccatccgta
agatgcttttctgtgactggtgagtactcaaccaagtcattctga
gaatagtgtatgcggcgaccgagttgctcttgcccggcgtcaata
cgggataataccgcgccacatagcagaactttaaaagtgctcatc
attggaaaacgttcttcggggcgaaaactctcaaggatcttaccg
ctgttgagatccagttcgatgtaacccactcgtgcacccaactga
tcttcagcatcttttactttcaccagcgtttctgggtgagcaaaa
acaggaaggcaaaatgccgcaaaaaagggaataagggcgacacgg
aaatgttgaatactcatactcttcctttttcaatattattgaagc
atttatcagggttattgtctcatgagcggatacatatttgaatgt
atttagaaaaataaacaaataggggttccgcgcacatttccccga
aaagtgccacctgacgtc.

### 3. Viral Vector

Gene editing compositions for modifying the genome of a cell include a viral vector. Typically, the transposon is contained within a viral vector containing a nucleic acid sequence that encodes one or more transposons. In preferred forms, the viral vector is an adeno-associated viral (AAV) vector.

a. Adeno-Associated Virus Vector

A preferred vector for inclusion in the gene editing compositions or for providing elements of the gene editing compositions (e.g., transposon) is an adeno-associated viral (AAV) vector.

AAV is a non-pathogenic, single-stranded DNA virus that has been actively employed for delivering therapeutic genes in both in vitro and in vivo systems (Choi, et al., Curr. Gene Ther., 5:299-310, (2005)). AAV is a replication-deficient virus that belongs to the parvovirus family, and is dependent on co-infection with other viruses, mainly adenoviruses, to replicate. Initially distinguished serologically, molecular cloning of AAV genes has identified hundreds of unique AAV strains in numerous species. Each end of the single-stranded DNA genome contains an inverted terminal repeat (ITR), which is the only cis-acting element required for genome replication and packaging. The single-stranded AAV genome contains three genes, Rep (Replication), Cap (Capsid), and aap (Assembly). These three genes give rise to at least nine gene products through the use of three promoters, alternative translation start sites, and differential splicing. These coding sequences are flanked by the ITRs. The Rep gene encodes four proteins (Rep78, Rep68, Rep52, and Rep40), while Cap expression gives rise to the viral capsid proteins (VP; VP1/VP2/VP3), which form the outer capsid shell that protects the viral genome, as well as being actively involved in cell binding and internalization. It is estimated that the viral coat includes 60 proteins, arranged into an icosahedral structure with the capsid proteins in a molar ratio of 1:1:10 (VP1:VP2:VP3).

Recombinant AAV (rAAV), which lacks viral DNA, is essentially a protein-based nanoparticle engineered to traverse the cell membrane, where it can ultimately traffic and deliver its DNA cargo into the nucleus of a cell. In the absence of Rep proteins, ITR-flanked transgenes encoded within rAAV can form circular concatemers that persist as episomes in the nucleus of transduced cells. Because recombinant episomal DNA does not integrate into host genomes, it will eventually be diluted over time as the cell undergoes repeated rounds of replication. This will eventually result in the loss of the transgene and transgene expression, with the rate of transgene loss dependent on the turnover rate of the transduced cell. These characteristics make rAAV ideal for certain gene therapy applications.

AAV can be advantageous over other viral vectors due to low toxicity (this can be due to the purification method not requiring ultra centrifugation of cell particles that can activate the immune response) and low probability of causing insertional mutagenesis because AAV does not integrate into the host genome (primarily remaining episomal).

Other suitable viral vectors include, without limitation, vectors derived from bacteriophages, baculoviruses, retroviruses (such as lentiviruses), adenoviruses, poxviruses, and Epstein-Barr viruses. In some forms, the viral vector is derived from a DNA virus (e.g., dsDNA or ssDNA virus) or an RNA virus (e.g., an ssRNA virus).

Numerous vectors and expression systems are commercially available from commercial vendors including Addgene, Novagen (Madison, WI), Clontech (Palo Alto, CA), Stratagene (La Jolla, CA), and Invitrogen/Life Technologies (Carlsbad, CA).

The AAV vector used in the disclosed compositions and methods can be a naturally occurring serotype of AAV including, but not limited to, AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64R1, rAAV2-retro, AAV-DJ, AAV-PHP.B, AAV-PHP.S, AAV-PHP.eB, or other engineered versions of AAV. In preferred forms, the AAV used in the disclosed compositions and methods is AAV6 or AAV9.

Twelve natural serotypes of AAV have thus far been identified, with the best characterized and most commonly used being AAV2. These serotypes differ in their tropism, or the types of cells they infect, making AAV a very useful system for preferentially transducing specific cell types. For example, AAV serotypes 1, 2, 5 or a hybrid capsid AAV1, AAV2, AAV5 or any combination thereof can be used for targeting brain or neuronal cells; AAV4 can be selected for targeting cardiac cells. AAV8 is useful for delivery to the liver cells. Researchers have further refined the tropism of AAV through pseudotyping, or the mixing of a capsid and genome from different viral serotypes. These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, AAV2/5 targets neurons that are not efficiently transduced by AAV2/2, and is distributed more widely in the brain, indicating improved transduction efficiency.

Other engineered AAVs have also been developed and can be used for the purpose of introducing transgenes, and in the disclosed compositions and methods. These are well known in the art and are contemplated for use in the disclosed methods and compositions.

One of skill in the art would be able to determine the optimal AAV serotype to be used for the respective application. The AAV can be AAV1, AAV2, AAV3, AAV4, AAV5, AAV6, AAV7, AAV8, AAV9, AAV10, AAV11, AAV12, artificial variants such as AAV.rhlO, AAV.rh32/33, AAV.rh43, AAV.rh64R1, rAAV2-retro, AAV-DJ, AAV-PHP.B, AAV-PHP.S, and AAV-PHP.eB, or combinations thereof. In preferred forms, the AAV vector for inclusion in the gene editing compositions or for providing elements of the gene editing compositions (e.g., transposon) is AAV6 or AAV9.

In some forms, more than one (e.g., 2, 3, 4, 5, 6, 10 or more) AAV vectors are introduced to the same target cell.

An exemplary nucleic acid sequence for a vector for use with the AAV system is (the transposon sequence is indicated in bold):

(SEQ ID NO: 43)
cctgcaggcagctgcgcgctcgctcgctcactgaggccgcccggg
caaagcccgggcgtcgggcgacctttggtcgcccggcctcagtga
gcgagcgagcgcgcagagagggagtggccaactccatcactaggg
gttcctgcggccgcacgcgttctagactatacagttgaagtcgga
agtttacatacacttaagttggagtcattaaaactcgtttttcaa
ctactccacaaatttcttgttaacaaacaatagttttggcaagtc
agttaggacatctactttgtgcatgacacaagtcatttttccaac
aattgtttacagacagattatttcacttataattcactgtatcac
aattccagtgggtcagaagtttacatacactaagttgactgtgcc
tttaaacagcttggaaaattccagaaaatgatgtcatggctttag
agaggatcctaggtcttgaaaggagtgggaattggctccggtgcc
cgtcagtgggcagagcgcacatcgcccacagtccccgagaagttg
gggggaggggtcggcaattgatccggtgcctagagaaggtggcgc
ggggtaaactgggaaagtgatgtcgtgtactggctccgccttttt
cccgagggtgggggagaaccgtatataagtgcagtagtcgccgtg
aacgttctttttcgcaacgggtttgccgccagaacacaggaccgg
ttctagacgtacggccaccatgggaattcttgagtgtatgtaaac
ttctgacccactgggaatgtgatgaaagaaataaaagctgaaatg
aatcattctctctactattattctgatatttcacattcttaaaat
aaagtggtgatcctaactgacctaagacagggaatttttactagg
attaaatgtcaggaattgtgaaaaagtgagtttaaatgtatttgg
ctaaggtgtatgtaaacttccgacttcaactgtataggcatgcgg
taaccacgtgcggaccgagcggccgcaggaacccctagtgatgga
gttggccactccctctctgcgcgctcgctcgctcactgaggccgg
gcgaccaaaggtcgcccgacgcccgggctttgcccgggcggcctc
agtgagcgagcgagcgcgcagctgcctgcaggggcgcctgatgcg
gtattttctccttacgcatctgtgcggtatttcacaccgcatacg
tcaaagcaaccatagtacgcgccctgtagcggcgcattaagcgcg
gcgggtgtggtggttacgcgcagcgtgaccgctacacttgccagc
gccctagcgcccgctcctttcgctttcttcccttcctttctcgcc
acgttcgccggctttccccgtcaagctctaaatcgggggctccct
ttagggttccgatttagtgctttacggcacctcgaccccaaaaaa
cttgatttgggtgatggttcacgtagtgggccatcgccctgatag
acggtttttcgccctttgacgttggagtccacgttctttaatagt
ggactcttgttccaaactggaacaacactcaaccctatctcgggc
tattcttttgatttataagggattttgccgatttcggcctattgg
ttaaaaaatgagctgatttaacaaaaatttaacgcgaattttaac
aaaatattaacgtttacaattttatggtgcactctcagtacaatc
tgctctgatgccgcatagttaagccagccccgacacccgccaaca
cccgctgacgcgccctgacgggcttgtctgctcccggcatccgct
tacagacaagctgtgaccgtctccgggagctgcatgtgtcagagg
ttttcaccgtcatcaccgaaacgcgcgagacgaaagggcctcgtg
atacgcctatttttataggttaatgtcatgataataatggtttct
tagacgtcaggtggcacttttcggggaaatgtgcgcggaacccct
atttgtttatttttctaaatacattcaaatatgtatccgctcatg
agacaataaccctgataaatgcttcaataatattgaaaaaggaag
agtatgagtattcaacatttccgtgtcgcccttattccctttttt
gcggcattttgccttcctgtttttgctcacccagaaacgctggtg
aaagtaaaagatgctgaagatcagttgggtgcacgagtgggttac
atcgaactggatctcaacagcggtaagatccttgagagttttcgc
cccgaagaacgttttccaatgatgagcacttttaaagttctgcta
tgtggcgcggtattatcccgtattgacgccgggcaagagcaactc
ggtcgccgcatacactattctcagaatgacttggttgagtactca
ccagtcacagaaaagcatcttacggatggcatgacagtaagagaa
ttatgcagtgctgccataaccatgagtgataacactgcggccaac
ttacttctgacaacgatcggaggaccgaaggagctaaccgctttt
ttgcacaacatgggggatcatgtaactcgccttgatcgttgggaa
ccggagctgaatgaagccataccaaacgacgagcgtgacaccacg
atgcctgtagcaatggcaacaacgttgcgcaaactattaactggc
gaactacttactctagcttcccggcaacaattaatagactggatg
gaggcggataaagttgcaggaccacttctgcgctcggcccttccg
gctggctggtttattgctgataaatctggagccggtgagcgtggg
tctcgcggtatcattgcagcactggggccagatggtaagccctcc
cgtatcgtagttatctacacgacggggagtcaggcaactatggat
gaacgaaatagacagatcgctgagataggtgcctcactgattaag
cattggtaactgtcagaccaagtttactcatatatactttagatt
gatttaaaacttcatttttaatttaaaaggatctaggtgaagatc
ctttttgataatctcatgaccaaaatcccttaacgtgagttttcg
ttccactgagcgtcagaccccgtagaaaagatcaaaggatcttct
tgagatcctttttttctgcgcgtaatctgctgcttgcaaacaaaa
aaaccaccgctaccagcggtggtttgtttgccggatcaagagcta
ccaactctttttccgaaggtaactggcttcagcagagcgcagata
ccaaatactgtccttctagtgtagccgtagttaggccaccacttc
aagaactctgtagcaccgcctacatacctcgctctgctaatcctg
ttaccagtggctgctgccagtggcgataagtcgtgtcttaccggg
ttggactcaagacgatagttaccggataaggcgcagcggtcgggc
tgaacggggggttcgtgcacacagcccagcttggagcgaacgacc
tacaccgaactgagatacctacagcgtgagctatgagaaagcgcc
acgcttcccgaagggagaaaggcggacaggtatccggtaagcggc
agggtcggaacaggagagcgcacgagggagcttccagggggaaac
gcctggtatctttatagtcctgtcgggtttcgccacctctgactt
gagcgtcgatttttgtgatgctcgtcaggggggcggagcctatgg
aaaaacgccagcaacgcggcctttttacggttcctggccttttgc
tggccttttgctcacatgt.

b. Other Viral Vector

In some forms, the vector for inclusion in the gene editing compositions or for providing elements of the gene editing compositions (e.g., transposon) is a viral vector such as a vesicular stomatitis (VSV) vector, a Bocavirus vector, such as a human bocavirus 1 (HBoV1) vector, a Herpes simplex virus (HSV) vector, or an adenovirus vector (AdV).

In some forms, the viral vector is a Herpes simplex virus (HSV) vector. Herpes simplex viruses (HSV) are large, enveloped dsDNA viruses characteristic of their lytic and latent nature of infection, which result in life-long latent infection of neurons and allows for long-term transgene expression. Deletion of HSV genes has generated expression vectors with low toxicity and an excellent packaging capacity of >30 kb foreign DNA. In some forms, the viral vector is a Vesicular stomatitis virus (VSV) vector. Vesicular stomatitis virus is a non-segmented, negative-stranded RNA virus that belongs to the family Rhabdoviridae, genus Vesiculovirus. VSV infects a broad range of animals, including cattle, horses, and swine. The genome of the virus codes for five major proteins, glycoprotein (G), matrix protein (M), nucleoprotein (N), large protein (L), and phosphoprotein (P). The G protein mediates both viral binding and host cell fusion with the endosomal membrane following endocytosis. The L and P proteins are subunits of the viral RNA-dependent RNA polymerase. The simple structure and rapid high-titer growth of VSV in mammalian and many other cells has made recombinant VSV a useful tool in the fields of cellular and molecular biology and virology.

In some forms, the viral vector is a human Bocavirus vector (HBoV). Exemplary human bocavirus vectors include human bocaviruses 1-4 (HBoV1-4), As well as Gorilla BoV.

In other forms, the viral vector is an adenovirus vector. In some forms, the vector is a chimeric vector, such as a vector that is based on a chimeric virus formed from a combination of one or more components from two or more different viral vectors. An exemplary chimeric viral vector is a chimeric bocavirus/adeno-associated virus vector. Therefore, in some forms, the vector is a chimeric HBoVt/AAV2 vector (e.g., rAAV2/HBoV1 chimeras).

### B. Cells

The gene editing compositions and methods are configured to achieve genomic modification of any cell type. For example, the cell can be a prokaryotic cell or a eukaryotic cell. In some forms, the cell is a fungal cell, such as a yeast cell. In some forms, the cell is an algal cell.

In some forms, the cell is a eukaryotic cell, such as a mammalian cell. In some forms the mammalian cell is derived from or is designed to be administered into a non-human mammal, e.g., primate, bovine, ovine, porcine, canine, rodent, Leporidae such as monkey, cow, sheep, pig, dog, rabbit, rat or mouse cell. In some forms, the cell is derived from a non-mammalian eukaryotic cell such as poultry bird (e.g., chicken), vertebrate fish (e.g., salmon) or shellfish (e.g., oyster, claim, lobster, shrimp) cell. In some forms, the cell is a plant cell. For example, in some forms, the cell is derived from a monocot or dicot of a crop or grain plant, such as cassava, corn, sorghum, soybean, wheat, oat or rice, or from a tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lactuca; plants of the genus Spinacia; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.).

In preferred forms, the cell to be modified is a human cell including, but not limited to, skin cells, lung cells, heart cells, kidney cells, pancreatic cells, muscle cells, neuronal cells, human embryonic stem cells, and pluripotent stem cells. More preferably, the cell to be modified can be a T cell (e.g., CD8+ T cells such as effector T cells, memory T cells, central memory T cells, and effector memory T cells, or CD4+ T cells such as Th1 cells, Th2 cells, Th17 cells, and Treg cells), hematopoietic stem cell (HSC), macrophage, natural killer cell (NK), or dendritic cell (DC).

In some forms, the cell is from an established cell line, or a primary cell. The term “primary cell,” refers to cells and cell cultures derived from a subject and allowed to grow in vitro for a limited number of passages, i.e. splitting, of the culture.

### 1. Sources of Human Cells

In preferred forms, the cells are obtained from a human subject. For example, In some forms, the cells are autologous cells, i.e., cells obtained from a subject prior to genetic modification and re-introduction to the same subject following modification. In other forms, the cells are heterologous cells, i.e., cells obtained from a different subject than the intended recipient. In some forms, the cells are frozen prior to or after genetic modification. Methods and compositions for freezing and thawing viable eukaryotic cells are known in the art. In some forms, the cells are autologous immune cells, such as T cells or progenitor cells/stem cells.

In some forms, cells are obtained from a healthy subject. In other forms, cells are obtained from a subject identified as having or at risk of having a disease or disorder, such as cancer and/or an auto-immune disease.

a. Sources of T Cells

In some forms, the cells are human immune cells, such as T cells. Therefore, in some forms, prior to expansion and genetic modification, T cells are obtained from a diseased or healthy subject. T cells can be obtained from a number of samples, including peripheral blood mononuclear cells, bone marrow, lymph node tissue, cord blood, thymus tissue, tissue from a site of infection, ascites, pleural effusion, spleen tissue, and tumors. In some forms, T cells are obtained from a unit of blood collected from a subject using any number of techniques known to the skilled artisan, such as FICOLL™ separation. In one preferred form, cells from the circulating blood of an individual are obtained by apheresis. The apheresis product typically contains lymphocytes, including T cells, monocytes, granulocytes, B cells, other nucleated white blood cells, red blood cells, and platelets. The cells collected by apheresis can be washed to remove the plasma fraction and to place the cells in an appropriate buffer or media for subsequent processing steps. In some forms, the cells are washed with phosphate buffered saline (PBS). In some forms, the wash solution lacks calcium and can lack magnesium or can lack many if not all divalent cations. After washing, the cells can be resuspended in a variety of biocompatible buffers, such as, for example, Ca2+-free, Mg2+-free PBS, PLASMALYTE A, or other saline solution with or without buffer. Alternatively, the undesirable components of the apheresis sample are removed and the cells directly resuspended in culture media.

In some forms, T cells are isolated from peripheral blood lymphocytes by lysing the red blood cells and depleting the monocytes, for example, by centrifugation through a PERCOLL™ gradient or by counterflow centrifugal elutriation. In specific forms, a specific subpopulation of T cells, such as CD3+, CD28+, CD4+, CD8+, CD45RAA+, and CD45RO+ T cells, is further isolated by positive or negative selection techniques. For example, in some forms, T cells are isolated by incubation with anti-CD3/anti-CD28 (i.e., 3×28)-conjugated beads, such as DYNABEADS® M-450 CD3/CD28 T, for a time period sufficient for positive selection of the desired T cells.

### 2. Genetically Modified (Transgenic) Cells

Cells modified according to the described compositions and systems for genetic modification are described.

In some forms the cell expresses one or more genes of interest that were introduced to the cell in a transposon delivered to the cell by transduction with a viral vector containing the transposon. In some forms, the cell expresses one or more gene products that were not expressed in the same cell prior to transduction by a viral vector containing a transposon including the gene and electroporation with transposase mRNA.

In preferred forms, the genetically modified cell is a human cell, such as a human immune cell. In some forms, the genetically modified cell is a human immune cell. In some forms, the genetically modified cell is a human T cell modified to express a chimeric antigen receptor (CAR T cell). In a particular form, the genetically modified cell is a human T cell modified to express a CAR (CAR T cell) specific for CD22, CD19, or both CD22 and CD19. In some forms, a plurality of genetically modified cells are combined with excipients and/or other reagents suitable for administration to a subject in the form of a “living drug” or therapeutic agent. In some forms, a plurality of cells genetically modified to express a chimeric antigen receptor are combined with excipients and/or other reagents suitable for administration to a subject to provide a T cell therapy for a subject in need thereof. In some forms, compositions containing CAR T cells include between about 10⁴ and about 10⁹ cells per kg body weight of the intended recipient (i.e, between 7×10⁵ and 7×10¹⁰ cells for an average adult), preferably 10⁵ to 10⁷ cells/kg body weight, including all integer values within those ranges.

### C. Pharmaceutical Compositions

Pharmaceutical compositions containing a genetically modified cell, or a population of genetically modified cells are provided. In some forms, the pharmaceutical compositions include one or more of a pharmaceutically acceptable buffer, carrier, diluent or excipients. In some forms, the pharmaceutical compositions include a specific number or population of cells, for example, expanded by culturing and expanding an isolated genetically modified cell (e.g., CAR T cell), e.g., a homogenous population. Therefore, in some forms, pharmaceutical compositions include a homogenous population of modified cells. In other forms, the pharmaceutical compositions include populations of cells that contain variable or different genetically modified cells, e.g., a heterogeneous population. In some forms, the pharmaceutical compositions include cells that are bispecific or multi-specific. In some forms, the cells have been isolated from a diseased or healthy subject prior to genetic modification. Introduction of gene editing compositions (e.g., mRNA encoding transposase and the one or more AAV vectors including a transposon) to the cell can be performed ex vivo.

The term “Pharmaceutically acceptable carrier” describes a pharmaceutically acceptable material, composition, or vehicle that is involved in carrying or transporting a compound of interest from one tissue, organ, or portion of the body to another tissue, organ, or portion of the body. For example, in some forms the carrier is a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

In some forms, pharmaceutical compositions include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The pharmaceutical compositions can be formulated for delivery via any route of administration. The term “Route of administration” can refer to any administration pathway known in the art, including but not limited to aerosol, nasal, oral, intravenous, intramuscular, intraperitoneal, inhalation, transmucosal, transdermal, parenteral, implantable pump, continuous infusion, topical application, capsules and/or injections. The pharmaceutical compositions are preferably formulated for intravenous administration.

Typically, the disclosed pharmaceutical compositions are administered in a manner appropriate to a disease to be treated (or prevented). The quantity and frequency of administration is typically determined by such factors as the condition of the patient, and the type and severity of the patient's disease, although appropriate dosages can be determined by clinical trials.

The disclosed pharmaceutical compositions can be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### III. Methods

Methods for providing genetically modified cells are provided. Typically, the methods introduce user-defined genetic modifications into eukaryotic cells in a controllable and highly efficient manner. In some forms, the methods facilitate immune cell engineering. For example, in some forms the methods provide genetically modified immune cells for production, research and development of cell therapy.

A composite gene delivery system for highly efficient engineering of therapeutic immune cells has been established. This system, termed MAJESTIC (mRNA AAV-Sleeping-Beauty Joint Engineering of Stable Therapeutic Immune Cells), combines the merits of mRNA, AAV vector, and transposon into one composite system. In MAJESTIC, the mRNA component encodes a transposase that mediates a pulse of genomic integration of the Sleeping Beauty (SB) transposon, which carries genes-of-interest and is embedded within the AAV vector. This system can transduce diverse immune cell types with minimal cellular toxicity, leading to highly efficient and stable therapeutic cargo delivery. As demonstrated in the Examples, the MAJESTIC system showed higher cell viability, chimeric antigen receptor (CAR) transgene expression, therapeutic cell yield, as well as prolonged transgene expression as compared with conventional gene delivery systems, such as lentiviral vector or DNA transposon/transposase electroporation alone. This system also demonstrated versatility for engineering different cell therapy constructs such as canonical CAR, bi-specific CAR, kill switch CAR and synthetic TCR; and for CAR delivery into various immune cells including T cell, natural killer cells, myeloid cells and induced pluripotent stem cells.

In some forms, the methods include one or more steps of introducing a gene of interest into a cell, including introducing:
(i) a viral vector including a transposon encoding the gene of interest; and
(ii) mRNA including a sequence that encodes one or more transposase enzymes configured to specifically mediate targeted integration of the transposon into the cellular genome,
wherein the mRNA is introduced to the cell via electroporation.
   A. Methods of CAR-T Generation with MAJESTIC Systems

Chimeric antigen receptor (CAR) T cells have recently become powerful players in the arsenal of immune-based cancer therapy. More recently, gene-editing technologies have enabled more direct engineering of immune cells. However, current lentiviral, retroviral, or CRISPR/Cas9 based methods have various limitations in CAR targeting efficiency and modularity, especially for generation of multi-component CAR T cells. Therefore, methods for cellular genome engineering that permit simple, efficient, and versatile permutations of combinatorial or simultaneous knockout and knock-in genomic modifications are provided. In particular, the AAV-SB transposon method which uses a combination of viral transposon delivery and non-viral (electroporation) delivery of transposase in the form of mRNA to the same cell to generate a stable transgenic cell (e.g., CAR-T cell) at high efficiency in one step is provided.

In preferred forms, the mRNA is introduced to the cell via electroporation. Electroporation is temporary destabilization of the cell membrane by insertion of a pair of electrodes into it so that DNA molecules in the surrounding media of the destabilized membrane would be able to penetrate into cytoplasm and nucleoplasm of the cell. The mRNA encoding transposase can also be introduced via direct electroporation.

Advantages of this AAV-transposon/mRNA electroporation method include, but are not limited to, design simplicity, higher delivery efficiency, lower toxicity, reduced exhaustion, increased effector function, and long term CAR enrichment (e.g., compared to standard approaches such as lentiviral CRISPR/Cas9 based approaches). This system can be used by, for example, both the scientific and clinical community for CAR-T research and production.

Gene editing compositions can be introduced to the cells in different compositions at the same time, or the gene editing compositions can be introduced to cells separated by a period of time from one or more minutes, hours, days or weeks. For example, in some forms, cells can be introduced to an mRNA encoding transposase enzyme via electroporation, followed by a vector (e.g., AAV vector) containing a transposon including a sequence that encodes one or more genes of interest. Alternatively, in other forms, cells can be first introduced to a vector (e.g., AAV vector) containing a transposon including a sequence that encodes one or more genes of interest, followed by electroporation to introduce mRNA encoding transposase enzyme. In some forms, a vector (e.g., AAV vector) containing a transposon including a sequence that encodes one or more genes of interest is introduced to cells at the same time as, or immediately before or after electroporation to introduce mRNA encoding transposase enzyme.

In some forms, the cells are isolated from a diseased or healthy subject prior to genetic modification. Therefore, in some forms, where the cells are intended to be genetically modified for use as therapeutic cells in a subject, the methods include one or more steps for obtaining cells from a subject who is the intended recipient of the modified cells. Methods for obtaining live cells from a subject for the purposes of genetic manipulation are known in the art.

In some forms, the methods include one or more steps to increase the number or population of cells, for example, by culturing and expanding an isolated genetically modified cell (e.g., CAR T cell), e.g., a homogenous population.

The following exemplary methods provides example materials and protocols that can be used to implement and use the disclosed systems.

1. Construction of AAV-SB Vector and SB100x Transposase mRNA

The methods include one or more steps to construct an AAV-SB vector.

In some forms, the methods introduce one or more CAR sequences into cells.

In an exemplary form, to create hybrid AAV-SB-CAR vector, the AAV-SB-CRISPR vector is used as a backbone, which has an sgRNA/SB100x expression cassette nested between SB arms and AAV ITRs. The sgRNA/SB100x expression cassette is replaced between the U6 promoter and the short polyA sequence with a CAR, or other expression cassettes (e.g. CD22, BCMA). CAR sequences are obtained via either:
(1) PCR amplification of CAR sequences from existing CAR constructs; or
(2) IDT gene synthesis (gBlock).

An exemplary method for generation of CD22BBz CAR is as previously described in Haso, et al., Blood., 121(7):1165-74 (2013).

An exemplary method generates a CD22 binding scFV (m971) specific for the human CD22 followed by CD8 hinge-transmembrane-regions linked to 4-1BB (CD137) intracellular domains and CD3ζ intracellular domain.

Another exemplary method generates a CD19 binding scFv (FMC63) (which can be found from NCBI (GenBank: HM852952)) and is followed by CD8 hinge-transmembrane-regions linked to 4-1BB (CD137) intracellular domains and CD3ζ intracellular domain, as described in Kochenderfer, et al., J. Immunother., 32(7):689-702 (2009)).

In some forms, the methods include one or more steps to provide transposase mRNA via in vitro mRNA transcription. The SB100x transposase is cloned into the Nco I and Hind III restriction endonuclease sites of the empty vector pcDNA3.1, which is used for in vitro transcription of mRNA.

For example, in an exemplary method, SB100x mRNA is transcribed from the SB100x plasmid using the HiScribe T7 ARCA mRNA (with tailing) Kit (NEB). Following RNA transcription, DNase treatment, and poly-A tailing, RNA purification is conducted using the Monarch RNA Cleanup Kit (50 ug) (NEB). After the concentration of the product was measured via Nandrop (with default RNA settings), the RNA is aliquoted and stored in −80° C.

In some forms, the methods thaw one or more frozen samples of mRNA encoding transposase enzyme on ice shortly before use in electroporation.

### 2. Preparation of Cells

The methods include one or more steps to obtain isolated cells for genomic modification. In some forms, the cells are obtained from an intended recipient. Therefore, in some forms, the methods include one or more steps to harvest viable cells from a live subject, for example, from a biological sample, such as blood or bone marrow. Methods for obtaining biological sample including cells from a subject are known in the art.

In some forms, the cells are one or more selected from HEK293T, NALM6, MM.1R, MCF7, NK-92, THP-1, human CD14+ monocytes, human PBMC, and human iPSC. These cells are available from multiple commercial sources, including, for example, ThermoFisher, American Type Culture Collection (ATCC), and STEMCELL.

In an exemplary method, HEK293T and MCF7 cells are cultured in DMEM (Gibco) media supplemented with 10% FBS (CORNING) and 200 U/mL penicillin-streptomycin (Gibco) (referred to as D10).

In an exemplary method, NALM6 and MM.1R cells are cultured in RPMI-1640 (Gibco) media supplemented with 10% FBS and 200 U/mL penicillin-streptomycin.

In an exemplary method, NK-92 cells are cultured in Alpha Minimum Essential medium (MEM) (Gibco) supplemented with 12.5% horse serum, 12.5% FBS, 0.2 mM inositol, 0.1 mM 2-mercaptoethanol, 0.02 mM folic acid, and 200 U/mL human IL-2.

In an exemplary method, THP-1 and CD14+ monocytes are cultured in RPMI-1640 media supplemented with 10% FBS, 1% Glutamax, and 1% penicillin-streptomycin. 20 ng/mL of human GM-CSF (BioLegend) is used to differentiate monocytes into macrophages.

In an exemplary method, human PBMCs are cultured in X-VIVO™ 15 media (Lonza) supplied with 5% human AB serum and IL-2.

In an exemplary method, Human iPSCs are cultured in StemFlex™ Medium (Gibco).

### 3. AAV Production, Purification, and Titration

The methods include one or more steps to obtain, purify and titrate viral vectors for use in the methods of introducing transposon nucleic acids to cells.

In exemplary forms, the methods prepare HEK293T cells in 150 mm-dishes. D10 media is replaced by 13 mL pre-warmed DMEM (FBS-free), and for each 150 mm-dish, HEK293T cells are transiently transfected with 5.2 μg transfer, 8.9% g AAV6 serotype and 10.4 μg pDF6 plasmids, which are pre-mixed with 130 μL of PEI (1 mg/mL) in 450 μL Opti-MEM medium. After 6 h of transfection, DMEM is replaced with 20 mL pre-warmed D10 media. Transfected cells are dislodged and collected in 50 mL Falcon tubes 72 h post-transfection for AAV purification.

In an exemplary method, AAV purification is performed according to methods known in the art, and viral titer is measured via RT-qPCR with a Taqman probe targeting the EFS sequence in the AAV vector.

An exemplary method for AAV purification and titration includes the following steps:
(i) Mix transfected cells with pure chloroform (1/10 volume).
(ii) Incubate cells at 37° C. with vigorous shaking for 1 hour.
(iii) Add NaCl to a final concentration of 1 M.
(iv) Centrifuge at 20,000 g at 4° C. for 15 minutes.
(v) Transfer aqueous layer to another tube and discard the chloroform layer.
(vi) Add PEG8000 to the sample until 10% (w/v) and shake until dissolved.
(vii) Incubate the mixture at 4° C. for 1 hour and then centrifuge at 20,000 g at 4° C.
(viii) Discard supernatant and suspend the pellet in DPBS with MgCl2.
(ix) Treat the sample with universal nuclease and incubate at 37° C. for 30 minutes.
(x) Add chloroform (1:1 volume), shake and centrifuge at 12,000 g at 4° C. for 15 minutes.
(xi) Isolate the aqueous layer and concentrate through a 100-kDa MWCO.
   Important step: concentrate AAV at high concentration so the volume can be reduced when performing the infection, which can decrease the toxicity of AAV.

AAV should be aliquoted and stored at −80° C.
(xii) Titer virus by qPCR using custom Taqman assays (ThermoFisher) targeted to promoter U6.

### 4. Human Primary T Cell, NK92, Macrophage, THP-1 Electroporation

The methods include one or more steps to introduce mRNA encoding transposase enzyme to cells by electroporation.

Typically, before electroporation, cells are collected, washed, and counted. In an exemplary method, 5e⁵-3e⁶ cells are used per reaction, depending on the specific experiment. In some forms, the methods include providing between 0.1 g and 100 μg, inclusive, of transposase mRNA per million cells, for example between 1 g and 10 g, inclusive. In a preferred form, the methods administer about 1 g of SB100x mRNA. In an exemplary method, 100 μL Buffer R with the cells and SB100x mRNA mixture is loaded into a Neon Pipette. Typically, the methods include one or more steps to avoid the production of bubbles and/or remove bubbles from the mixture.

In an exemplary method, the electroporation parameter is set at about 1600 V, 10 ms, and 3 pulses for T cells, THP-1 cells, and NK-92 cells; 1900 V, 30 ms, and 1 pulse for macrophages. Cells are immediately transferred to a 24-well plate with pre-warmed media after electroporation.

In some forms, the methods introduce specific quantities of viral vector including transposon (e.g., AAV-SB) to the same cells at a time point 24 hours prior to or after electroporation. For example, in some forms, the methods introduce specific quantities of viral vector including transposon (e.g., AAV-SB) to the same cells at a time point that is between about 24 hours before to about 24 hours after electroporation, inclusive, such as between 10 hours before and 10 hours after electroporation. Therefore, in some forms, the methods introduce virus-transposon (e.g., AAV-SB) 1 hour, 2 hours, 3 hours, 4 hours, 5 hours, 6 hours, 7 hours, 8 hours or 9 hours before or after electroporation. In a preferred form, the methods introduce virus-transposon (e.g., AAV-SB) from 1 hour to 4 hours before electroporation. In a preferred from, the methods introduce virus-transposon (e.g., AAV-SB) from 1 hour to 4 hours before electroporation, most preferably about 4 hours before electroporation.

### 5. AAV Transduction

The methods include one or more steps to introduce the transposon into the cells via AAV viral transduction. In some forms, the methods first electroporate the cells (e.g., human CD4 and/or CD8 T cells) with transposase mRNA (e.g., SB100x mRNA), then transduced them with a titration series of AAV-SB virus. In some forms, the methods administer AAV to the cells in an amount that is a multiplicity of infection (MOI) of, for example, between 1E1 and 1E10, for example, 1E1, 1E2, 1E3, 1E4, 1E5, 1E6, 1E7, 1E8, 1E9 and 1E10. In an exemplary method, the amount of AAV-SB administered to the cells is between 1E3 and 1E5, such as 1E3 or 1E4.

### 6. CAR-T Detection by Flow Cytometry

In some forms, the methods include one or more steps to identify genetically-modified cells by flow-cytometry. In an exemplary method, T cells (or other immune cells) are collected and washed once with PBS. For CAR constructs lacking a Flag tag (e.g for CD22 and BCMA CARs), cells are incubated with CD22-Fc or BCMA-Fc protein in PBS for 30 min on ice, then stained with anti-human IgG Fc-PE and other immune markers antibodies and incubated on ice for 30 min. For CAR constructs containing a Flag tag, Fc protein incubation is skipped and Flag is stained directly with an anti-Flag antibody. For the CD19.20.CAR detection, cells are incubated with biotinylated protein L (R&D) on the ice for 30 min, then stained with APC streptavidin for 30 min on the ice. Cells are washed with MACS buffer (0.5% BSA and 2 mM EDTA in PBS) and then analyzed on a BD FACSAria cytometer. Data analysis is typically performed using techniques known in the art. In exemplary forms, the methods employ software such as FlowJo software 9.9.4 (Threestar, Ashland, OR).

### 7. Equipment

Any of the described methods can utilize any of the following equipment:
(i) PCR Thermocycler
(ii) Tissue culture hood
(iii) 15-cm tissue culture dishes (Corning)
(iv) Retronectin-coated plates (Takara)
(v) An electroporation system (e.g., Neon® (ThermoFisher), Nucleofactor (Lonza), or ATX/GTX (Maxcyte))
(vi) Bioanalyzer (Agilent)
(vii) Pipettes and tips
(viii) Next generation sequencing machines (Illumina)
(ix) Cell culture incubators (37° C., 5% CO₂)
(x) Countess automated cell counter (Thermo Fisher)
(xi) Plate reader (PerkinElmer)
(xii) BD FACSAria II (BD Biosciences)
(xiii) FlowJo software 9.9.4 (Treestar, Ashland, OR)

### IV. Methods of Treatment

Methods of treatment including cells and other therapeutic agents produced according to the MAJESTIC system are described. In preferred embodiments, the methods include Adoptive Cell Therapy (ACT) employing cells prepared according to the described methods for genetic manipulation of cells.

An exemplary method involves treating a subject (e.g., a human) having a disease, disorder, or condition by administering to the subject an effective amount of a pharmaceutical composition including genetically-modified cells prepared according to the MAJESTIC system. In some forms, the methods treat a subject having a disease, disorder, or condition associated with an elevated expression or specific expression of an antigen by administering to the subject an effective amount of a pharmaceutical composition including cells modified according to the disclosed methods. In some forms, the methods treat a subject having a disease, disorder, or condition associated with an elevated expression or specific expression of an antigen by administering to the subject an effective amount of a pharmaceutical composition including T cells modified to contain a CAR that targets the antigen.

Methods of treating a subject having a disease, disorder, or condition including administering to the subject an effective amount of a pharmaceutical composition including live, viable cells engineered to express a gene of interest are provided. In some forms, the methods treat a subject having a disease, disorder, or condition associated with an elevated expression or specific expression of an antigen. In some forms, the methods include administering to the subject an effective amount of a T cell modified to express a CAR that targets the antigen. For example, in some forms, the methods treat a subject having a disease, disorder, or condition by administering to the subject an effective amount of a pharmaceutical composition having a genetically modified cell, where the cell is modified by introducing to the cell
(i) a viral vector including a transposon encoding the gene of interest; and
(ii) mRNA including a sequence that encodes one or more transposase enzymes configured to specifically mediate targeted integration of the transposon into the cellular genome,
wherein the mRNA is introduced to the cell via electroporation.
   The cell can have been isolated from the subject having the disease, disorder, or condition, or from a healthy donor, prior to genetic modification.

### A. Diseases to be Treated

Methods of treating a subject having a disease or disorder are provided. The subject to be treated can have a disease, disorder, or condition such as but not limited to, cancer, an immune system disorder such autoimmune disease, an inflammatory disease, a neuronal disorder, HIV/AIDS, diabetes, a cardiovascular disease, an infectious disease, or combinations thereof. The disease, disorder, or condition can be associated with an elevated expression or specific expression of an antigen. In some forms, the methods treat or prevent cancer and/or autoimmune disease in a subject in need thereof.

### 1. Cancer

In some forms, the methods treat or prevent a cancer in a subject.

Cancer is a disease of genetic instability, allowing a cancer cell to acquire the hallmarks proposed by Hanahan and Weinberg, including (i) self-sufficiency in growth signals; (ii) insensitivity to anti-growth signals; (iii) evading apoptosis; (iv) sustained angiogenesis; (v) tissue invasion and metastasis; (vi) limitless replicative potential; (vii) reprogramming of energy metabolism; and (viii) evading immune destruction (Cell, 144:646-674, (2011)).

Tumors, which can be treated in accordance with the disclosed methods, are classified according to the embryonic origin of the tissue from which the tumor is derived. Carcinomas are tumors arising from endodermal or ectodermal tissues such as skin or the epithelial lining of internal organs and glands. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

Table 2 provides a non-limiting list of cancers for which the CAR of the disclosed methods and compositions can target a specific or an associated antigen.

TABLE 2

| | | | | |
|---|---|---|---|---|
| Acute | Acute Myeloid | Adrenocortical | AIDS-Related | Kaposi Sarcoma |
| Lymphoblastic | Leukemia | Carcinoma | Cancers | |
| Leukemia | (AML) | | | |
| (ALL) | | | | |
| AIDS-Related | Primary CNS | Anal Cancer | Appendix | Astrocytomas |
| Lymphoma | Lymphoma | | Cancer | |
| | | | (Gastrointestinal | |
| | | | Carcinoid | |
| | | | Tumors) | |
| Atypical | Brain Cancer | Basal Cell | Bile Duct | Bladder Cancer |
| Teratoid/ | | Carcinoma of the | Cancer | |
| Rhabdoid | | Skin | | |
| Tumor | | | | |
| Bone Cancer | Brain Tumors | Breast Cancer | Bronchial | Burkitt |
| (includes | | | Tumors | Lymphoma |
| Ewing Sarcoma | | | | |
| and | | | | |
| Osteosarcoma | | | | |
| and Malignant | | | | |
| Fibrous | | | | |
| Histiocytoma) | | | | |
| Non-Hodgkin | Carcinoid | Carcinoma of | Cardiac (Heart) | Embryonal |
| Lymphoma | Tumors | Unknown Primary | Tumors | Tumors |
| Germ Cell | Primary CNS | Cervical Cancer | Cholangio- | Chordoma |
| Tumor | Lymphoma | | carcinoma | |
| Chronic | Chronic | Chronic | Colorectal | Cranio- |
| Lymphocytic | Myelogenous | Myeloproliferative | Cancer | pharyngioma |
| Leukemia | Leukemia | Neoplasms | | |
| (CLL) | (CML) | | | |
| Cutaneous | Ductal | Endometrial Cancer | Ependymoma | Esophageal |
| T-Cell | Carcinoma | | | Cancer |
| Lymphoma | In Situ | | | |
| (Mycosis | (DCIS) | | | |
| Fungoides and | | | | |
| Sézary | | | | |
| Syndrome) | | | | |
| Esthesioneuro- | Ewing Sarcoma | Extracranial Germ | Eye Cancer | Intraocular |
| blastoma | | Cell Tumor | | Melanoma |
| Fallopian Tube | Fibrous | Osteosarcoma | Gallbladder | Gastric Cancer |
| Cancer | Histiocytoma of | | Cancer | |
| | Bone | | | |
| Stomach | Gastrointestinal | Gastrointestinal | Central | Extracranial |
| Cancer | Carcinoid | Stromal Tumors | Nervous | Germ Cell |
| | Tumor | (GIST) | System Germ | Tumors |
| | | | Cell Tumors | |
| Extragonadal | Ovarian Germ | Testicular Cancer | Gestational | Hairy Cell |
| Germ Cell | Cell Tumors | | Trophoblastic | Leukemia |
| Tumors | | | Disease | |
| Head and Neck | Heart Tumors | Hepatocellular | Histiocytosis | Hodgkin |
| Cancer | | (Liver) Cancer | (Langerhans | Lymphoma |
| | | | Cell) | |
| Hypopharyngeal | Intraocular | Islet Cell Tumors | Pancreatic | Kidney Cancer |
| Cancer | Melanoma | | Neuroendocrine | |
| | | | Tumors | |
| Renal Cell | Langerhans | Laryngeal Cancer | Leukemia | Lip and Oral |
| Cancer | Cell | | | Cavity Cancer |
| | Histiocytosis | | | |
| Liver Cancer | Lung Cancer | Lymphoma | Male Breast | Malignant |
| | (Non-Small | | Cancer | Fibrous |
| | Cell and Small | | | Histiocytoma of |
| | Cell) | | | Bone and |
| | | | | Osteosarcoma |
| Melanoma | Intraocular | Merkel Cell | Malignant | Metastatic |
| | (Eye) | Carcinoma (Skin | Mesothelioma | Cancer |
| | Melanoma | Cancer) | | |
| Metastatic | Midline Tract | Mouth Cancer | Multiple | Multiple |
| Squamous | Carcinoma | | Endocrine | Myeloma/Plasma |
| Neck Cancer | With NUT | | Neoplasia | Cell |
| with Occult | Gene Changes | | Syndromes | Neoplasms |
| Primary | | | | |
| Mycosis | Myelodysplastic | Myelodysplastic/ | Nasal Cavity | Nasopharyngeal |
| Fungoides | Syndromes | Myeloproliferative | and Paranasal | Cancer |
| (Lymphoma) | | Neoplasms | Sinus Cancer | |
| Neuroblastoma | Non-Small Cell | Oral Cancer | Oropharyngeal | Ovarian Cancer |
| | Lung Cancer | | Cancer | |
| Pancreatic | Papillomatosis | Paraganglioma | Paranasal Sinus | Parathyroid |
| Cancer | | | and Nasal | Cancer |
| | | | Cavity Cancer | |
| Penile Cancer | Pharyngeal | Pheochromocytoma | Pituitary | Plasma Cell |
| | Cancer | | Tumor | Neoplasm/ |
| | | | | Multiple |
| | | | | Myeloma |
| Pleuropulmonary | Primary Central | Primary Peritoneal | Prostate Cancer | Rectal Cancer |
| Blastoma | Nervous | Cancer | | |
| | System (CNS) | | | |
| | Lymphoma | | | |
| Recurrent | Retinoblastoma | Rhabdomyosarcoma | Salivary Gland | Sarcoma |
| Cancer | | | Cancer | |
| Vascular | Uterine | Sézary Syndrome | Small Cell | Small Intestine |
| Tumors | Sarcoma | (Lymphoma) | Lung Cancer | Cancer |
| Soft Tissue | Squamous Cell | Stomach (Gastric) | Throat Cancer | Thymoma |
| Sarcoma | Carcinoma | Cancer | | |
| Thymic | Thyroid Cancer | Transitional Cell | Carcinoma of | Ureter and |
| Carcinoma | | Cancer of the Renal | Unknown | Renal Pelvis |
| | | Pelvis and Ureter | Primary | |
| Transitional | Urethral Cancer | Uterine Cancer | Vaginal Cancer | Vulvar Cancer |
| Cell Cancer | | | | |
| Wilms Tumor | | | | |

The disclosed compositions and methods can be used in the treatment of one or more cancers provided in Table 4.

The disclosed compositions and methods of treatment thereof are generally suited for treatment of carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth.

The types of cancer that can be treated with the provided compositions and methods include, but are not limited to, cancers such as vascular cancer such as multiple myeloma, adenocarcinomas and sarcomas, of bone, bladder, brain, breast, cervical, colorectal, esophageal, kidney, liver, lung, nasopharangeal, pancreatic, prostate, skin, stomach, and uterine. In some forms, the compositions are used to treat multiple cancer types concurrently. The compositions can also be used to treat metastases or tumors at multiple locations.

Exemplary tumor cells include, but are not limited to, tumor cells of cancers, including leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as, but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenstram's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytom and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, and carcinosarcoma. For a review of such disorders, see Fishman et al., 1985, Medicine, 2d Ed., J.B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

### 2. Immune System Disorders

In some forms, the methods treat or prevent one or more immune system disorders, including autoimmune diseases.

Under certain circumstances, the ability of the immune system to distinguish self from foreign antigens can be misdirected against healthy tissues, resulting in the undesirable attack and destruction of normal host cells (i.e., autoimmune diseases). Autoimmune diseases include over 100 types of diseases, with varied etiology and prognoses based on factors such as the affected region, the age of onset, response to the therapeutic agents and clinical manifestation may vary among different people (Muhammad, et al., Chimeric Antigen Receptor Based Therapy as a Potential Approach in Autoimmune Diseases: How Close Are We to the Treatment, Frontiers in Immunology, 11 (2020)).

Auto-antibody-secreting B lymphocytes and self-reactive T-lymphocytes play a key role in the development of autoimmune diseases. Based on the extent of tissue damage, autoimmunity is classified into two general categories, including organ-specific and systemic autoimmune. The former involves a specific area of the body such as type I diabetes (T1D), multiple sclerosis (MS), rheumatoid arthritis (RA), inflammatory bowel diseases (IBDs), and myasthenia gravis (MG), while the latter affects multiple regions of the body, causing systemic lupus erythematosus (SLE) and Sjögren's syndrome (SS). Therefore, in some forms, the methods treat or prevent one or more organ-specific autoimmune diseases in a subject. In other forms, the methods treat or prevent one or more systemic autoimmune diseases in a subject.

In some forms, the methods reduce or prevent one or more physiological processes associated with the development or progression of autoimmune disease in a subject. For example, in some forms, the methods reduce or prevent one or more of epitope spreading, for example, where infections alter the primary epitope into the secondary epitope or form several neoepitopes on antigen-presenting cells; bystander activation or pre-primed autoreactive T cell activation in a T cell receptor (TCR)-independent manner; persistent virus infection, or the constant presence of viral antigens that prompt immune responses; or immunological cross-reactivity between a host and pathogen, for example, due to shared immunologic epitopes or sequence similarities.

a. Immune System Disorder to be Treated

Non-limiting examples of immune system disorders include 22q11.2 deletion syndrome, Achondroplasia and severe combined immunodeficiency, Adenosine Deaminase 2 deficiency, Adenosine deaminase deficiency, Adult-onset immunodeficiency with anti-interferon-gamma autoantibodies, Agammaglobulinemia, non-Bruton type, Aicardi-Goutieres syndrome, Aicardi-Goutieres syndrome type 5, Allergic bronchopulmonary aspergillosis, Alopecia, Alopecia totalis, Alopecia universalis, Amyloidosis AA, Amyloidosis familial visceral, Ataxia telangiectasia, Autoimmune lymphoproliferative syndrome, Autoimmune lymphoproliferative syndrome due to CTLA4 haploinsuffiency, Autoimmune polyglandular syndrome type 1, Autosomal dominant hyper IgE syndrome, Autosomal recessive early-onset inflammatory bowel disease, Autosomal recessive hyper IgE syndrome, Bare lymphocyte syndrome 2, Barth syndrome, Blau syndrome, Bloom syndrome, Bronchiolitis obliterans, C1q deficiency, Candidiasis familial chronic mucocutaneous, autosomal recessive, Cartilage-hair hypoplasia, CHARGE syndrome, Chediak-Higashi syndrome, Cherubism, Chronic atypical neutrophilic dermatosis with lipodystrophy and elevated temperature, Chronic graft versus host disease, Chronic granulomatous disease, Chronic Infantile Neurological Cutaneous Articular syndrome, Chronic mucocutaneous candidiasis (CMC), Cohen syndrome, Combined immunodeficiency with skin granulomas, Common variable immunodeficiency, Complement component 2 deficiency, Complement component 8 deficiency type 1, Complement component 8 deficiency type 2, Congenital pulmonary alveolar proteinosis, Cryoglobulinemia, Cutaneous mastocytoma, Cyclic neutropenia, Deficiency of interleukin-1 receptor antagonist, Dendritic cell, monocyte, B lymphocyte, and natural killer lymphocyte deficiency, Dyskeratosis congenital, Dyskeratosis congenita autosomal dominant, Dyskeratosis congenita autosomal recessive, Dyskeratosis congenita X-linked, Epidermodysplasia verruciformis, Familial amyloidosis, Finnish type, Familial cold autoinflammatory syndrome, Familial Mediterranean fever, Familial mixed cryoglobulinemia, Felty's syndrome, Glycogen storage disease type 1B, Griscelli syndrome type 2, Hashimoto encephalopathy, Hashimoto's syndrome, Hemophagocytic lymphohistiocytosis, Hennekam syndrome, Hepatic venoocclusive disease with immunodeficiency, Hereditary folate malabsorption, Hermansky Pudlak syndrome 2, Herpes simplex encephalitis, Hoyeraal Hreidarsson syndrome, Hyper IgE syndrome, Hyper-IgD syndrome, ICF syndrome, Idiopathic acute eosinophilic pneumonia, Idiopathic CD4 positive T-lymphocytopenia, IL12RB1 deficiency, Immune defect due to absence of thymus, Immune dysfunction with T-cell inactivation due to calcium entry defect 1, Immune dysfunction with T-cell inactivation due to calcium entry defect 2, Immunodeficiency with hyper IgM type 1, Immunodeficiency with hyper IgM type 2, Immunodeficiency with hyper IgM type 3, Immunodeficiency with hyper IgM type 4, Immunodeficiency with hyper IgM type 5, Immunodeficiency with thymoma, Immunodeficiency without anhidrotic ectodermal dysplasia, Immunodysregulation, polyendocrinopathy and enteropathy X-linked, Immunoglobulin A deficiency 2, Intestinal atresia multiple, IRAK-4 deficiency, Isolated growth hormone deficiency type 3, Kawasaki disease, Large granular lymphocyte leukemia, Leukocyte adhesion deficiency type 1, LRBA deficiency, Lupus, Lymphocytic hypophysitis, Majeed syndrome, Melkersson-Rosenthal syndrome, MHC class 1 deficiency, Muckle-Wells syndrome, Multifocal fibrosclerosis, Multiple sclerosis, MYD88 deficiency, Neonatal systemic lupus erythematosus, Netherton syndrome, Neutrophil-specific granule deficiency, Nijmegen breakage syndrome, Omenn syndrome, Osteopetrosis autosomal recessive 7, Palindromic rheumatism, Papillon Lefevre syndrome, Partial androgen insensitivity syndrome, PASLI disease, Pearson syndrome, Pediatric multiple sclerosis, Periodic fever, aphthous stomatitis, pharyngitis and adenitis, PGM3-CDG, Poikiloderma with neutropenia, Pruritic urticarial papules plaques of pregnancy, Purine nucleoside phosphorylase deficiency, Pyogenic arthritis, pyoderma gangrenosum and acne, Relapsing polychondritis, Reticular dysgenesis, Sarcoidosis, Say Barber Miller syndrome, Schimke immunoosseous dysplasia, Schnitzler syndrome, Selective IgA deficiency, Selective IgM deficiency, Severe combined immunodeficiency, Severe combined immunodeficiency due to complete RAG1/2 deficiency, Severe combined immunodeficiency with sensitivity to ionizing radiation, Severe combined immunodeficiency, Severe congenital neutropenia autosomal recessive 3, Severe congenital neutropenia X-linked, Shwachman-Diamond syndrome, Singleton-Merten syndrome, SLC35C1-CDG (CDG-IIc), Specific antibody deficiency, Spondyloenchondrodysplasia, Stevens-Johnson syndrome, T-cell immunodeficiency, congenital alopecia and nail dystrophy, TARP syndrome, Trichohepatoenteric syndrome, Tumor necrosis factor receptor-associated periodic syndrome, Twin to twin transfusion syndrome, Vici syndrome, WHIM syndrome, Wiskott Aldrich syndrome, Woods Black Norbury syndrome, X-linked agammaglobulinemia, X-linked lymphoproliferative syndrome, X-linked lymphoproliferative syndrome 1, X-linked lymphoproliferative syndrome 2, X-linked magnesium deficiency with Epstein-Barr virus infection and neoplasia, X-linked severe combined immunodeficiency, and ZAP-70 deficiency.

The disclosed compositions and methods can also be used to treat autoimmune diseases or disorders. Exemplary autoimmune diseases or disorders, which are not mutually exclusive with the immune system disorders described above, include Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Bald disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjagren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, Vogt-Koyanagi-Harada Disease, and Wegener's granulomatosis (or Granulomatosis with Polyangiitis (GPA)).

### 3. Other Disease or Disorders

In some forms the methods treat one or more additional disease or disorder in a subject in need thereof. For example, in some forms the methods treat one or more genetic disease or disorders in a subject, such as a hereditary genetic disease or disorder, or a somatic genetic disease or disorder in a subject.

Any of the methods can include treating a subject having an underlying disease or disorder. For example, in some forms, the methods treat a disease or disorder, such as a cancer or auto-immune disease in a patient having another disease or disorder, such as diabetes, a bacterial infection (e.g., Tuberculosis), viral infection (e.g., Hepatitis, HIV, HPV infection, etc.), or a drug-associated disease or disorder. In some forms, the methods treat an immunocompromised subject. In some forms, the methods treat a subject having a disease of the kidney, liver, heart, lung, brain, bladder, reproductive system, bowel/intestines, stomach, bones or skin.

### B. Effective Amounts

The effective amount or therapeutically effective amount of a pharmaceutical compositions including modified cells, such as therapeutic T cells, can be a dosage sufficient to treat, inhibit, or alleviate one or more symptoms of a disease or disorder, such as a cancer or autoimmune disease, or to otherwise provide a desired pharmacologic and/or physiologic effect, for example, reducing, inhibiting, or reversing one or more of the underlying pathophysiological mechanisms underlying a disease or disorder, such as cancer or autoimmune disease.

In some forms, when administration of the pharmaceutical compositions including modified cells, such as therapeutic T cells, elicits an anti-cancer response, the amount administered can be expressed as the amount effective to achieve a desired anti-cancer effect in the recipient. For example, in some forms, the amount of the pharmaceutical compositions including modified cells, such as therapeutic T cells, is effective to inhibit the viability or proliferation of cancer cells in the recipient. In some forms, the amount of the pharmaceutical composition including modified cells, such as therapeutic T cells, is effective to reduce the tumor burden in the recipient, or reduce the total number of cancer cells, and combinations thereof. In other forms, the amount of the pharmaceutical compositions including modified cells, such as therapeutic T cells, is effective to reduce one or more symptoms or signs of cancer in a cancer patient, or signs of an autoimmune disease in a patient having an autoimmune disease or disorder. Signs of cancer can include cancer markers, such as PSMA levels in the blood of a patient.

The effective amount of the pharmaceutical compositions including modified cells, such as therapeutic T cells, that is required will vary from subject to subject, depending on the species, age, weight and general condition of the subject, the severity of the disorder being treated, and its mode of administration. Thus, it is not possible to specify an exact amount for every pharmaceutical composition. However, an appropriate amount can be determined by one of ordinary skill in the art using only routine experimentation given the teachings herein. For example, effective dosages and schedules for administering the pharmaceutical compositions including therapeutic T cells can be determined empirically, and making such determinations is within the skill in the art. In some forms, the dosage ranges for the administration of the compositions including therapeutic T cells are those large enough to effect reduction in cancer cell proliferation or viability, or to reduce tumor burden for example.

The dosage should not be so large as to cause adverse side effects, such as unwanted cross-reactions, anaphylactic reactions, and the like. Generally, the dosage will vary with the age, condition, and sex of the patient, route of administration, whether other drugs are included in the regimen, and the type, stage, and location of the disease to be treated. The dosage can be adjusted by the individual physician in the event of any counter-indications. It will also be appreciated that the effective dosage of the composition including therapeutic T cells used for treatment can increase or decrease over the course of a particular treatment. Changes in dosage can result and become apparent from the results of diagnostic assays.

Dosage can vary, and can be administered in one or more dose administrations daily, for one or several days. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

It can generally be stated that a pharmaceutical composition containing CAR T cells described herein can be administered at a dosage of 10⁴ to 10⁹ cells/kg body weight, preferably 10⁵ to 10⁷ cells/kg body weight, including all integer values within those ranges. In some forms, patients can be treated by infusing a disclosed pharmaceutical composition containing CAR expressing cells (e.g., T cells) in the range of about 10⁴ to 10¹² or more cells per square meter of body surface (cells/m).

The infusion can be repeated as often and as many times as the patient can tolerate until the desired response is achieved. CAR T cell compositions can also be administered once or multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In some forms, the unit dosage is in a unit dosage form for intravenous injection. In some forms, the unit dosage is in a unit dosage form for oral administration. In some forms, the unit dosage is in a unit dosage form for inhalation. In some forms, the unit dosage is in a unit dosage form for intra-tumoral injection.

Treatment can be continued for an amount of time sufficient to achieve one or more desired therapeutic goals, for example, a reduction of the amount of cancer cells relative to the start of treatment, or complete absence of cancer cells in the recipient. Treatment can be continued for a desired period of time, and the progression of treatment can be monitored using any means known for monitoring the progression of anti-cancer treatment in a patient. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one year, two years, three years, or up to five years.

The efficacy of administration of a particular dose of the pharmaceutical compositions including modified cells, such as therapeutic T cells, according to the methods described herein can be determined by evaluating the aspects of the medical history, signs, symptoms, and objective laboratory tests that are known to be useful in evaluating the status of a subject in need for the treatment of cancer or other diseases and/or conditions. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: (1) a subject's physical condition is shown to be improved (e.g., a tumor has partially or fully regressed), (2) the progression of the disease or condition is shown to be stabilized, or slowed, or reversed, or (3) the need for other medications for treating the disease or condition is lessened or obviated, then a particular treatment regimen will be considered efficacious. In some forms, efficacy is assessed as a measure of the reduction in tumor volume and/or tumor mass at a specific time point (e.g., 1-5 days, weeks, or months) following treatment.

### C. Modes of Administration

Any of the disclosed genetically modified cells (e.g., CAR T cells) can be used therapeutically in combination with a pharmaceutically acceptable carrier. The compositions described herein can be conveniently formulated into pharmaceutical compositions composed of one or more of the compounds in association with a pharmaceutically acceptable carrier. See, e.g., Remington's Pharmaceutical Sciences, latest edition, by E.W. Martin Mack Pub. Co., Easton, PA, which discloses typical carriers and conventional methods of preparing pharmaceutical compositions that can be used in conjunction with the preparation of formulations of the therapeutics described herein and which is incorporated by reference herein. These most typically would be standard carriers for administration of compositions to humans. In one aspect, for humans and non-humans, these include solutions such as sterile water, saline, and buffered solutions at physiological pH. Other therapeutics can be administered according to standard procedures used by those skilled in the art.

The pharmaceutical compositions including modified cells, such as therapeutic T cells, described herein can include, but are not limited to, carriers, thickeners, diluents, buffers, preservatives, surface active agents and the like in addition to the therapeutic(s) of choice.

Pharmaceutical compositions containing one or more modified cells, such as therapeutic T cells, and optionally one or more additional therapeutic agents can be administered to the subject in a number of ways depending on whether local or systemic treatment is desired, and on the area to be treated. Thus, for example, a pharmaceutical composition including modified cells, such as therapeutic T cells, can be administered as an intravenous infusion, or directly injected into a specific site, for example, into or surrounding a tumor. Moreover, a pharmaceutical composition can be administered to a subject as an ophthalmic solution and/or ointment to the surface of the eye, vaginally, rectally, intranasally, orally, by inhalation, or parenterally, for example, by intradermal, subcutaneous, intramuscular, intraperitoneal, intrarectal, intraarterial, intralymphatic, intravenous, intrathecal and intratracheal routes. In some forms, the compositions are administered directly into a tumor or tissue, e.g., stereotactically.

Parenteral administration, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., intraocular injection, intra-retinal injection, or sub-retinal injection); subcutaneous injection or deposition including subcutaneous infusion (such as by osmotic pumps); direct application by a catheter or other placement device (e.g., an implant including a porous, non-porous, or gelatinous material).

Preparations for parenteral administration include sterile aqueous or non-aqueous solutions, suspensions, and emulsions which can also contain buffers, diluents and other suitable additives. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Administration of the pharmaceutical compositions containing one or more genetically modified cells (e.g., CAR T cells) can be localized (i.e., to a particular region, physiological system, tissue, organ, or cell type) or systemic.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### D. Combination Therapy

Any of the disclosed pharmaceutical compositions including modified cells, such as therapeutic T cells (e.g., containing a population of CAR cells), can be used alone, or in combination with other therapeutic agents or treatment modalities, for example, chemotherapy or stem-cell transplantation. As used herein, “combination” or “combined” refer to either concomitant, simultaneous, or sequential administration of the therapeutics.

In some forms, the pharmaceutical compositions and other therapeutic agents are administered separately through the same route of administration. In other forms, the pharmaceutical compositions and other therapeutic agents are administered separately through different routes of administration. The combinations can be administered either concomitantly (e.g., as an admixture), separately but simultaneously (e.g., via separate intravenous lines into the same subject; one agent is given orally while the other agent is given by infusion or injection, etc.), or sequentially (e.g., one agent is given first followed by the second).

Examples of preferred additional therapeutic agents include other conventional therapies known in the art for treating the desired disease, disorder or condition. In some forms, the therapeutic agent is one or more other targeted therapies (e.g., a targeted cancer therapy) and/or immune-checkpoint blockage agents (e.g., anti-CTLA-4, anti-PD1, and/or anti-PDL1 agents such as antibodies).

The compositions and methods described herein may be used as a first therapy, second therapy, third therapy, or combination therapy with other types of therapies known in the art, such as chemotherapy, surgery, radiation, gene therapy, immunotherapy, bone marrow transplantation, stem cell transplantation, targeted therapy, cryotherapy, ultrasound therapy, photodynamic therapy, radio-frequency ablation or the like, in an adjuvant setting or a neoadjuvant setting.

The disclosed pharmaceutical compositions and/or other therapeutic agents, procedures or modalities can be administered during periods of active disease, or during a period of remission or less active disease. The pharmaceutical compositions can be administered before the additional treatment, concurrently with the treatment, post-treatment, or during remission of the disease or disorder. When administered in combination, the disclosed pharmaceutical compositions and the additional therapeutic agents (e.g., second or third agent), or all, can be administered in an amount or dose that is higher, lower or the same than the amount or dosage of each agent used individually, e.g., as a monotherapy. In certain forms, the administered amount or dosage of the disclosed pharmaceutical composition, the additional therapeutic agent (e.g., second or third agent), or all, is lower (e.g., at least 20%, at least 30%, at least 40%, or at least 50%) than the amount or dosage of each agent used individually, e.g., as a monotherapy (e.g., required to achieve the same therapeutic effect).

### 1. Additional Anti-Cancer Agents

In some forms, the methods administer one or more additional anti-cancer agents to a subject.

In the context of cancer, targeted therapies are therapeutic agents that block the growth and spread of cancer by interfering with specific molecules (“molecular targets”) that are involved in the growth, progression, and spread of cancer. Many different targeted therapies have been approved for use in cancer treatment. These therapies include hormone therapies, signal transduction inhibitors, gene expression modulators, apoptosis inducers, angiogenesis inhibitors, immunotherapies, and toxin delivery molecules. Numerous antineoplastic drugs can be used in combination with the disclosed pharmaceutical compositions. In some forms, the additional therapeutic agent is a chemotherapeutic or antineoplastic drug. The majority of chemotherapeutic drugs can be divided into alkylating agents, antimetabolites, anthracyclines, plant alkaloids, topoisomerase inhibitors, monoclonal antibodies, and other anti-tumor agents.

### 2. Additional Therapeutic Agents Against Autoimmune Diseases

In some forms, the methods also include administering one or more conventional therapies for autoimmune diseases to the subject.

Exemplary therapies for autoimmune diseases include immunosuppressive agents, such as steroids or cytostatic drugs, analgesics, non-steroidal anti-inflammatory drugs, glucocorticoids, immunosuppressive and immunomodulatory agents, such as methotrexate, leflunomide, hydroxychloroquine, and sulfasalazine. In some forms, the methods administer one or more disease-modifying antirheumatic drugs (DMARDs). In some forms, the methods administer one or more biologic agents for localized treatment (i.e., agents that do not affect the entire immune system), such as TNF-α inhibitors, belimumab and rituximab depleting B cells, T-cell co-stimulation blocker, anti-interleukin 6 (IL-6), anti-IL-1, and protein kinase inhibitors. In other forms, the methods also administer one or more monoclonal antibodies (mAbs), such as anti-TNFα, anti-CD19, anti-CD20, anti-CD22, and anti-IL6R, or other mAbs that target multiple B cell subtypes, and other aberrant cells in autoimmune diseases.

### V. Kits

The compositions, reagents, and other materials for cellular genomic engineering can be packaged together in any suitable combination as a kit useful for performing, or aiding in the performance of, the methods. It is useful if the components in a given kit are designed and adapted for use together in the method. For example, kits with one or more compositions for administration to a subject, may include a pre-measured dosage of the composition in a sterile needle, ampule, tube, container, or other suitable vessel. The kits may include instructions for dosages and dosing regimens.

Provided are kits containing a transposon (e.g., SB transposon), an AAV vector, mRNA encoding a transposase enzyme (e.g., SB100X transposase) or a vector suitable of expressing the mRNA, and instructional material for use thereof. In preferred forms, the kit includes a plurality of vectors, where each vector independently contains a transposon encoding one or more genes for insertion into a host cell genome, such as a CAR expression cassette. In some forms, the kit contains a population of cells (e.g., T cells) collectively containing the AAV and/or transposon. The instructional material can include a publication, a recording, a diagram, or any other medium of expression which can be used to communicate the usefulness of the compositions and methods of the kit. For example, the instructional material may provide instructions for methods using the kit components, such as performing transfections, transductions, infections, and conducting screens.

In some forms, kits include a Sleeping Beauty transposon, such as the Sleeping Beauty SB100x hyperactive transposase. In some forms, kits include an Adeno-associated virus (AAV) vector. In some forms, kits include a transposon including a gene of interest having a reporter gene, a Chimeric Antigen Receptor (CAR), or combinations thereof. In some forms, kits include a transposon that includes a promoter and/or polyadenylation signal operationally linked to a reporter gene and/or a CAR; in some forms, the kit includes a transposon including a CAR that is specific for an antigen selected from the group including a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof; for example, in some forms the CAR targets one or more antigens selected from the group including AFP, AKAP 4, ALK, Androgen receptor, B7H3, BCMA, Bcr Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6 AML, FAP, Fos related antigen1, Fucosyl, fusion, GD2, GD3, GloboH, GM3, gp100, GPC3, HER 2/neu, HER2, HMWMAA, HPV E6/E7, hTERT, Idiotype, IL12, IL13RA2, IM19, IX, LCK, Legumain, lgK, LMP2, MAD CT 1, MAD CT 2, MAGE, MelanA/MART1, Mesothelin, MET, ML IAP, MUC1, Mutant p53, MYCN, NA17, NKG2D L, NY BR 1, NY ESO 1, NY ESO 1, OY TES1, p53, Page4, PAP, PAX3, PAX5, PD L1, PDGFR β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP 2, Tyrosinase, VEGFR2, WT1, and XAGE.

In some forms, the kit includes a transposon including a CAR that is specific for an antigen that is selected from a cancer antigen selected from 4 1BB, 5T4, adenocarcinoma antigen, alpha fetoprotein, BAFF, B lymphoma cell, C242 antigen, CA 125, carbonic anhydrase 9 (CA IX), C MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA 4, DR5, EGFR, EpCAM, CD3, FAP, fibronectin extra domain B, folate receptor 1, GD2, GD3 ganglioside, glycoprotein 75, GPNMB, HER2/neu, HGF, human scatter factor receptor kinase, IGF 1 receptor, IGF I, IgG1, L1 CAM, IL 13, IL 6, insulin-like growth factor I receptor, integrin α5β1, integrin αvβ3, MORAb 009, MS4A1, MUC1, mucin CanAg, N glycolylneuraminic acid, NPC 1C, PDGF R a, PDL192, phosphatidylserine, prostatic carcinoma cells, RANKL, RON, ROR1, SCH 900105, SDC1, SLAMF7, TAG 72, tenascin C, TGF beta 2, TGF f, TRAIL R1, TRAIL R2, tumor antigen CTAA16.88, VEGF A, VEGFR 1, VEGFR2, and vimentin; in some forms, the CAR is bispecific or multivalent; in some forms, the CAR is anti CD19 or anti CD22, or both. Exemplary CARs include CD19BBz or CD22BBz.

In some forms, the kit includes mRNA encoding transposase that incorporates N6 methyladenosine (m6A), 5 methylcytosine (m5C), pseudouridine (Y), N1 methylpseudouridine (melW), 5 methoxyuridine (5moU), a 5′ cap, a poly(A) tail, one or more nuclear localization signals, or combinations thereof; in some forms, the mRNA, or the transposon, or both are codon optimized for expression in a eukaryotic cell. In exemplary forms, the kits include a viral vector that is AAV6 or AAV9, and/or cells. Exemplary cells include a T cell, hematopoietic stem cell (HSC), macrophage, natural killer cell (NK), or dendritic cell (DC). In some forms, the T cell is a CD8+ T cell selected from effector T cells, memory T cells, central memory T cells, and effector memory T cells. In some forms, the T cell is a CD4+ T cell selected from the group including Th1 cells, Th2 cells, Th17 cells, and Treg cells. The disclosed compositions and methods can be further understood through the following numbered paragraphs.

1. A method of introducing a gene of interest into a cell, the method comprising introducing to the cell:
(i) a viral vector comprising a transposon encoding the gene of interest; and
(ii) mRNA comprising a sequence that encodes one or more transposase enzymes configured to specifically mediate targeted integration of the transposon into the cellular genome,
   wherein the mRNA is introduced to the cell via electroporation.

2. The method of paragraph 1, wherein the transposon is the Sleeping Beauty transposon.

3. The method of paragraph 2, wherein the transposase enzyme is the Sleeping Beauty SB100x hyperactive transposase.

4. The method of any one of paragraphs 1-3, wherein the viral vector is an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector comprising a combination of any two or more of a Adeno-associated virus (AAV) vector, Herpes Simplex virus (HSV) vector, vesicular stomatitis (VSV) vector, or a human Bocavirus vector (hBoV).

5. The method of any one of paragraphs 1-4, wherein the transposon comprises a gene of interest comprising a reporter gene, a Chimeric Antigen Receptor (CAR), or combinations thereof.

6. The method of paragraph 5, wherein the transposon further comprises a promoter and/or polyadenylation signal operationally linked to the reporter gene and/or the CAR.

7. The method of paragraph 5 or 6, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

8. The method of any one of paragraphs 5-7, wherein the CAR targets one or more antigens selected from the group consisting of AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAX5, PD-L1, PDGFR-β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP-2, Tyrosinase, VEGFR2, WT1, and XAGE.

9. The method of paragraph 7, wherein the antigen is a cancer antigen selected from the group consisting of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

10. The method of any one of paragraphs 5-9, wherein the CAR is bispecific or multivalent.

11. The method of any one of paragraphs 5-10, wherein the CAR is anti-CD19 or anti-CD22, or both.

12. The method of paragraph 11, wherein the CAR is CD19BBz or CD22BBz.

13. The method of any one of paragraphs 1-12, wherein the mRNA comprises N6-methyladenosine (m6A), 5-methylcytosine (m5C), pseudouridine (Y), N1-methylpseudouridine (me1ψ), 5-methoxyuridine (5moU), a 5′ cap, a poly(A) tail, one or more nuclear localization signals, or combinations thereof.

14. The method of any one of paragraphs 1-13, wherein the mRNA, or the transposon, or both are codon optimized for expression in a eukaryotic cell.

15. The method of any one of paragraphs 1-14, wherein the mRNA encoding transposase and the viral vector is introduced to the cell at the same or different times.

16. The method of paragraph 15, wherein the mRNA is introduced to the cell by electroporation at a time point between 10 hours before, and 10 hours after the viral vector comprising a transposon encoding the gene of interest is introduced to the cell.

17. The method of paragraph 16, wherein the mRNA is introduced to the cell by electroporation at a time point between one and four hours before the viral vector comprising a transposon encoding the gene of interest is introduced to the cell.

18. The method of any one of paragraphs 4-17, wherein the AAV vectors is AAV6 or AAV9.

19. The method of any one of paragraphs 1-18, wherein the introduction is performed ex vivo.

20. The method of any one of paragraphs 1-19, wherein the cell is a T cell, hematopoietic stem cell (HSC), macrophage, natural killer cell (NK), or dendritic cell (DC).

21. The method of paragraph 20, wherein the T cell is a CD8+ T cell selected from the group consisting of effector T cells, memory T cells, central memory T cells, and effector memory T cells.

22. The method of paragraph 21, wherein the T cell is a CD4+ T cell selected from the group consisting of Th1 cells, Th2 cells, Th17 cells, and Treg cells.

23. An isolated cell modified according to the method of any one of paragraphs 1-22.

24. The isolated cell of paragraph 23, wherein the cell includes a gene of interest that is a CAR.

25. The isolated cell of paragraph 24, wherein the CAR is bispecific or multi-specific.

26. A population of cells derived by expanding the cell of any one paragraphs 23-25.

27. A pharmaceutical composition comprising the population of cells of paragraph 26 and a pharmaceutically acceptable buffer, carrier, diluent or excipient.

28. A method of treating a subject having a disease, disorder, or condition comprising administering to the subject an effective amount of the pharmaceutical composition of paragraph 27.

29. A method of treating a subject having a disease, disorder, or condition associated with an elevated expression or specific expression of an antigen, the method comprising administering to the subject an effective amount of a T cell modified according to the method of any one of paragraphs 5-22, wherein the T cell comprises a CAR that targets the antigen.

30. A method of treating a subject having a disease, disorder, or condition, the method comprising administering to the subject an effective amount of a pharmaceutical composition comprising a genetically modified cell, wherein the cell is genetically modified by a method comprising introducing to the cell:
(i) a viral vector comprising a transposon encoding the gene of interest; and
(ii) mRNA comprising a sequence that encodes one or more transposase enzymes configured to specifically mediate targeted integration of the transposon into the cellular genome,
   wherein the mRNA is introduced to the cell via electroporation.

31. The method of paragraph 30, wherein the transposon is the Sleeping Beauty transposon.

32. The method of paragraph 30 or 31, wherein the transposase enzyme is the SB100x hyperactive transposase.

33. The method of any one of paragraphs 30-32, wherein the viral vector is an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector comprising a combination of any two or more of a Adeno-associated virus (AAV) vector, Herpes Symplex virus (HSV) vector, vesicular stomatitis (VSV) vector, or a human Bocavirus vector (hBoV).

34. The method of any one of paragraphs 30-33, wherein the transposon comprises a gene of interest comprising a reporter gene, a chimeric antigen receptor (CAR), or combinations thereof.

35. The method of paragraph 34, wherein the transposon further comprises a promoter and/or polyadenylation signal operationally linked to the reporter gene and/or the CAR.

36. The method of paragraph 34 or 35, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

37. The method of any one of paragraphs 34-36, wherein the CAR targets one or more antigens selected from the group comprising of AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAX5, PD-L1, PDGFR-β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP-2, Tyrosinase, VEGFR2, WT1, and XAGE.

38. The method of paragraph 37, wherein the antigen is a cancer antigen selected from the group consisting of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

39. The method of any one of paragraphs 34-38, wherein the CAR is bispecific or multivalent.

40. The method of any one of paragraphs 34-39, wherein the CAR is anti-CD19 or anti-CD22, or both.

41. The method of paragraph 40, wherein the CAR is CD19BBz or CD22BBz.

42. The method of any one of paragraphs 30-41, the AAV vectors is AAV6 or AAV9.

43. The method of any one of paragraphs 30-41, wherein the genetically modified cell is a T cell, hematopoietic stem cell (HSC), macrophage, natural killer cell (NK), or dendritic cell (DC).

44. The method of paragraph 43, wherein the T cell is a CD8+ T cell selected from the group consisting of effector T cells, memory T cells, central memory T cells, and effector memory T cells.

45. The method of paragraph 43, wherein the T cell is a CD4+ T cell selected from the group consisting of Th1 cells, Th2 cells, Th17 cells, and Treg cells.

46. The method of any one of paragraphs 34-45, wherein the introduction to the cell is performed ex vivo.

47. The method of paragraph 46, wherein the cell was isolated from the subject having the disease, disorder, or condition prior to the introduction to the cell.

48. The method of paragraph 46, wherein the cell was isolated from a healthy donor prior to the introduction to the cell.

49. The method of any one of paragraphs 30-48, wherein the pharmaceutical composition comprises a population of cells derived by expanding the genetically modified cell.

50. The method of any one of paragraphs 30-49, wherein the subject is a human.

51. A system for introducing a gene of interest into a cell, the system comprising:
(i) a viral vector comprising a transposon encoding the gene of interest; and
(ii) mRNA comprising a sequence that encodes one or more transposase enzymes configured to specifically mediate targeted integration of the transposon into the cellular genome.

52. The system of paragraph 51, wherein the transposon is the Sleeping Beauty transposon.

53. The system of paragraph 52, wherein the transposase enzyme is the Sleeping Beauty SB100x hyperactive transposase.

54. The system of any one of paragraphs 51-53, wherein the viral vector is an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector comprising a combination of any two or more of a Adeno-associated virus (AAV) vector, Herpes Simplex virus (HSV) vector, vesicular stomatitis (VSV) vector, or a human Bocavirus vector (hBoV).

55. The system of any one of paragraphs 51-54, wherein the transposon comprises a gene of interest comprising a reporter gene, a Chimeric Antigen Receptor (CAR), or combinations thereof.

56. The system of paragraph 55, wherein the transposon further comprises a promoter and/or polyadenylation signal operationally linked to the reporter gene and/or the CAR.

57. The system of paragraph 55 or 56, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

58. The system of any one of paragraphs 55-57, wherein the CAR targets one or more antigens selected from the group consisting of AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAX5, PD-L1, PDGFR-β, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP-2, Tyrosinase, VEGFR2, WT1, and XAGE.

59. The system of paragraph 57, wherein the antigen is a cancer antigen selected from the group consisting of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

60. The system of any one of paragraphs 55-59, wherein the CAR is bispecific or multivalent.

61. The system of any one of paragraphs 55-60, wherein the CAR is anti-CD19 or anti-CD22, or both.

62. The system of paragraph 61, wherein the CAR is CD19BBz or CD22BBz.

63. The system of any one of paragraphs 51-62, wherein the mRNA comprises N6-methyladenosine (m6A), 5-methylcytosine (m5C), pseudouridine (T), N1-methylpseudouridine (me1ψ), 5-methoxyuridine (5moU), a 5′ cap, a poly(A) tail, one or more nuclear localization signals, or combinations thereof.

64. The system of any one of paragraphs 51-63, wherein the mRNA, or the transposon, or both are codon optimized for expression in a eukaryotic cell.

65. The system of any one of paragraphs 54-64, wherein the AAV vectors is AAV6 or AAV9.

66. The system of any one of paragraphs 51-65, wherein the cell is a T cell, hematopoietic stem cell (HSC), macrophage, natural killer cell (NK), or dendritic cell (DC).

67. The system of paragraph 66, wherein the T cell is a CD8+ T cell selected from the group consisting of effector T cells, memory T cells, central memory T cells, and effector memory T cells.

68. The system of paragraph 67, wherein the T cell is a CD4+ T cell selected from the group consisting of Th1 cells, Th2 cells, Th17 cells, and Treg cells.

69. A kit for introducing a gene of interest into a cell, the kit comprising:
(i) a viral vector comprising a transposon encoding the gene of interest; and
(ii) mRNA comprising a sequence that encodes one or more transposase enzymes configured to specifically mediate targeted integration of the transposon into the cellular genome.

70. The kit of paragraph 69, wherein the transposon is the Sleeping Beauty transposon.

71. The kit of paragraph 70, wherein the transposase enzyme is the Sleeping Beauty SB100x hyperactive transposase.

72. The kit of any one of paragraphs 69-71, wherein the viral vector is an Adeno-associated virus (AAV) vector, or an adenovirus vector, or a Herpes Simplex virus (HSV) vector, or a vesicular stomatitis (VSV) vector, or a human Bocavirus vector (hBoV), or a chimeric vector comprising a combination of any two or more of a Adeno-associated virus (AAV) vector, Herpes Simplex virus (HSV) vector, vesicular stomatitis (VSV) vector, or a human Bocavirus vector (hBoV).

73. The kit of any one of paragraphs 69-72, wherein the transposon comprises a gene of interest comprising a reporter gene, a Chimeric Antigen Receptor (CAR), or combinations thereof.

74. The kit of paragraph 73, wherein the transposon further comprises a promoter and/or polyadenylation signal operationally linked to the reporter gene and/or the CAR.

75. The kit of paragraph 73 or 74, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

76. The kit of any one of paragraphs 73-75, wherein the CAR targets one or more antigens selected from the group consisting of AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, Mutant p53, MYCN, NA 17, NKG2D-L, NY-BR-1, NY-ESO-1, NY-ESO-1, OY-TES1, p53, Page4, PAP, PAX3, PAX5, PD-L1, PDGFR-0, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP-2, Tyrosinase, VEGFR2, WT1, and XAGE.

77. The kit of paragraph 75, wherein the antigen is a cancer antigen selected from the group consisting of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, and vimentin.

78. The kit of any one of paragraphs 73-77, wherein the CAR is bispecific or multivalent.

79. The kit of any one of paragraphs 73-78, wherein the CAR is anti-CD19 or anti-CD22, or both.

80. The kit of paragraph 79, wherein the CAR is CD19BBz or CD22BBz.

81. The kit of any one of paragraphs 69-80, wherein the mRNA comprises N6-methyladenosine (m6A), 5-methylcytosine (m5C), pseudouridine (T), N1-methylpseudouridine (me1ψ), 5-methoxyuridine (5moU), a 5′ cap, a poly(A) tail, one or more nuclear localization signals, or combinations thereof.

82. The kit of any one of paragraphs 69-81, wherein the mRNA, or the transposon, or both are codon optimized for expression in a eukaryotic cell.

83. The kit of any one of paragraphs 72-82, wherein the AAV vectors is AAV6 or AAV9.

84. The kit of any one of paragraphs 69-83, wherein the cell is a T cell, hematopoietic stem cell (HSC), macrophage, natural killer cell (NK), or dendritic cell (DC).

85. The kit of paragraph 84, wherein the T cell is a CD8+ T cell selected from the group consisting of effector T cells, memory T cells, central memory T cells, and effector memory T cells.

86. The kit of paragraph 85, wherein the T cell is a CD4+ T cell selected from the group consisting of Th1 cells, Th2 cells, Th17 cells, and Treg cells.

### EXAMPLES

### Example 1: Establishment of the MAJESTIC System and High Efficiency Generation of CAR-T Cells

The MAJESTIC system has two core components: 1) the AAV-SB vector carrying desired cell therapy transgenes (AAV-SB-CTx), and 2) the engineered mRNA encoding the SB transposase (mRNA-Transposase).

### Methods

This study received institutional regulatory approval. All recombinant DNA and biosafety work were performed under the guidelines of Yale Environment, Health and Safety (EHS) Committee with approved protocols (Chen-rDNA-15-45; Chen-rDNA-18-45). All human sample work was performed under the guidelines of Yale University Institutional Review Board (IRB) with an approved protocol (HIC #2000020784).

### Construction of AAV-SB-CAR Vector

The hybrid AAV-SB-CAR vectors were created based on the previously established AAV-SB-CRISPR vector as a backbone, which has an sgRNA/SB100x expression cassette nested between SB anns and AAV ITRs (see, Ye, et al. In vivo CRISPR screening in CD8 T cells with AAV-Sleeping Beauty hybrid vectors identifies membrane targets for improving immunotherapy for glioblastoma. Nat Biotechnol 37, 1302-1313, doi:10.1038/s41587-019-0246-4 (2019)). This expression cassette was replaced between the U6 promoter and the short polyA sequence with a CAR or other expression cassettes. As this study utilized multiple types of CARs (e.g. CD22, BCMA), each CAR sequence via was obtained either 1) PCR amplification of CAR sequences from existing CAR constructs in the lab or 2) IDT gene synthesis. The SB100x transposase was cloned into the Nco I and Hind III restriction endonuclease sites of the empty vector pcDNA3.1, which was used for in vitro transcription of mRNA.

### Cell Culture

HEK293T, NALM6, MM.1R, MCF7, NK-92, THP-1, human CD14+ monocytes, human PBMC, and human iPSC were purchased from commercial sources (ThermoFisher, American Type Culture Collection (ATCC), and STEMCELL).

HEK293T and MCF7 cells were cultured in DMEM (Gibco) media supplemented with 10% FBS (CORNING) and 200 U/mL penicillin-streptomycin (Gibco), hereafter referred to as D10. NALM6 and MM.1R cells were cultured in RPMI-1640 (Gibco) media supplemented with 10% FBS and 200 U/mL penicillin-streptomycin.

NK-92 cells were cultured in Alpha Minimum Essential medium (MEM) (Gibco) supplemented with 12.5% horse serum, 12.5% FBS, 0.2 mM inositol, 0.1 mM 2-mercaptoethanol, 0.02 mM folic acid, and 200 U/mL human IL-2. THP-1 and CD14+ monocytes were cultured in RPMI-1640 media supplemented with 10% FBS, 1% Glutamax, and 1% penicillin-streptomycin. 20 ng/mL of human GM-CSF (BioLegend) was used to differentiate monocytes into macrophages for 7 days, then macrophages were collected for electroporation and/or viral transduction, CAR-macrophage cells were maintained in RPMI complete media supplied with 20 ng/mL of human GM-CSF.

Human PBMCs CD4, and CD8 T cells were purchased from the StemCell and were cultured in X-VIVO™ 15 media (Lonza) supplied with 5% human AB serum and (MP Biomedicals) and 400 U/mL human IL-2. (BioLegend). T cells were activated with Dynabeads Human T-Activator CD3/CD28 (ThennoFisher) with a T cell:Beads ratio at 1:1. In this study, multiple T cell donors were involved in various experiments; the donors for each experiment are clarified in figure legends. Human iPSCs were cultured in StemFlex™ Medium (Gibco).

### AAV Production, Purification, and Titration

HEK293T cells were prepared in 150 mm-dishes as above. D10 media was replaced by 13 mL pre-warmed DMEM (FBS-free). For each 150 mm-dish, HEK293T cells were transiently transfected with 5.2 μg transfer, 8.9 μg AAV6 serotype and 10.4 μg pDF6 plasmids, which was pre-mixed with 130 μL of PEI (1 mg/mL) in 450 μL Opti-MEM medium. After 6 h of transfection, DMEM was replaced with 20 mL pre-warmed D10 media. Transfected cells were dislodged and collected in 50 mL Falcon tubes 72 h post-transfection for AAV purification. AAV purification was performed as previously reported. Viral titer was measured via RT-qPCR with a Taqman probe targeting the EFS sequence in the AAV vector.

### Flow Cytometry

T cells (or other immune cells) were collected and washed once with PBS. For CAR constructs lacking a Flag tag (e.g. for CD22 and BCMA CARs), cells were incubated with CD22-Fc or BCMA-Fc protein in PBS for 30 min on ice, then stained with anti-human IgG Fc-PE and other immune markers antibodies and incubated on ice for 30 min. For CAR constructs containing a Flag tag, Fc protein incubation was skipped and Flag was stained directly with an anti-Flag antibody. For the CD19.20.CAR detection, cells were incubated with biotinylated protein L (R&D) on the ice for 30 min, then stained with APC streptavidin for 30 min on the ice. Cells were washed with MACS buffer (0.5% BSA and 2 mM EDTA in PBS) and then analyzed on a BD FACSAria cytometer. Data analysis was performed using FlowJo software 9.9.4 (Threestar, Ashland, OR). All flow cytometry antibodies were purchased from BioLegend.

In Vitro mRNA Transcription

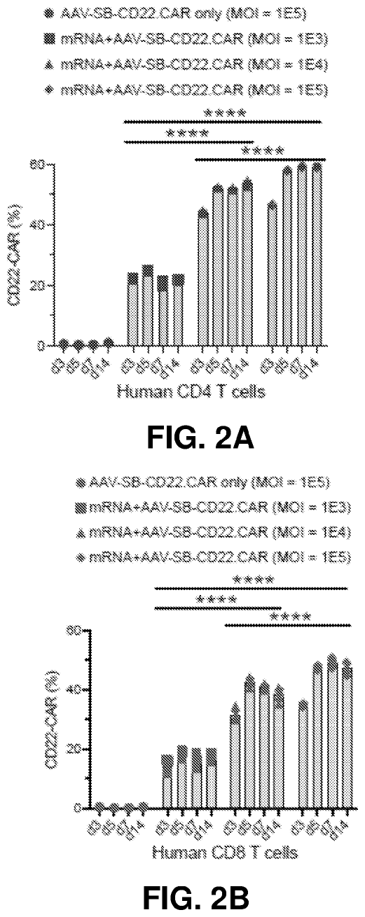

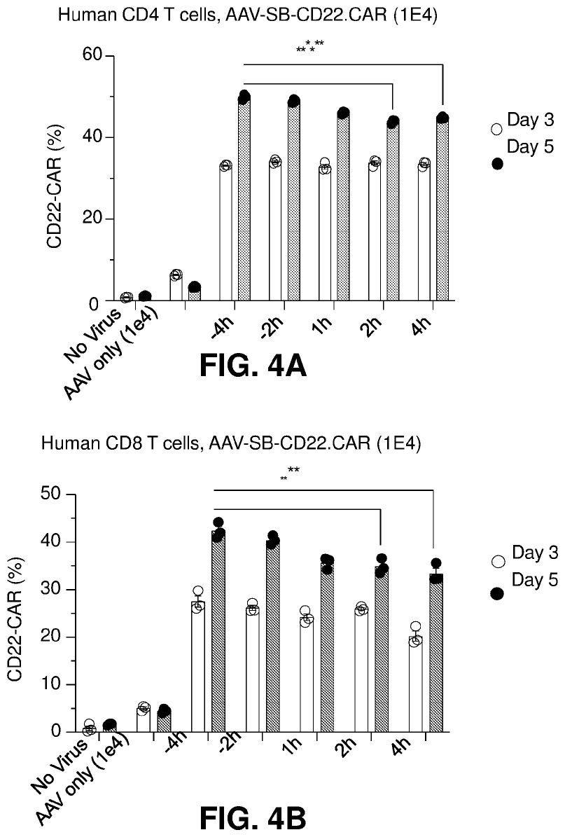

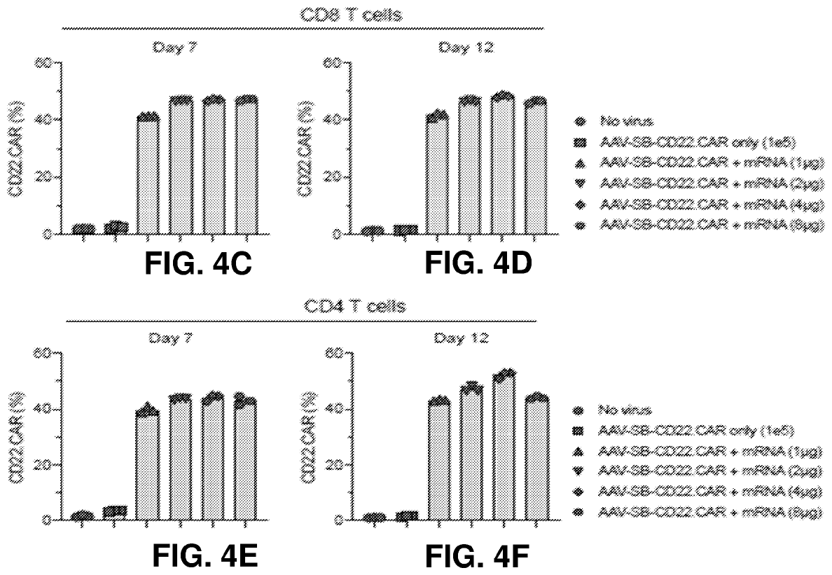

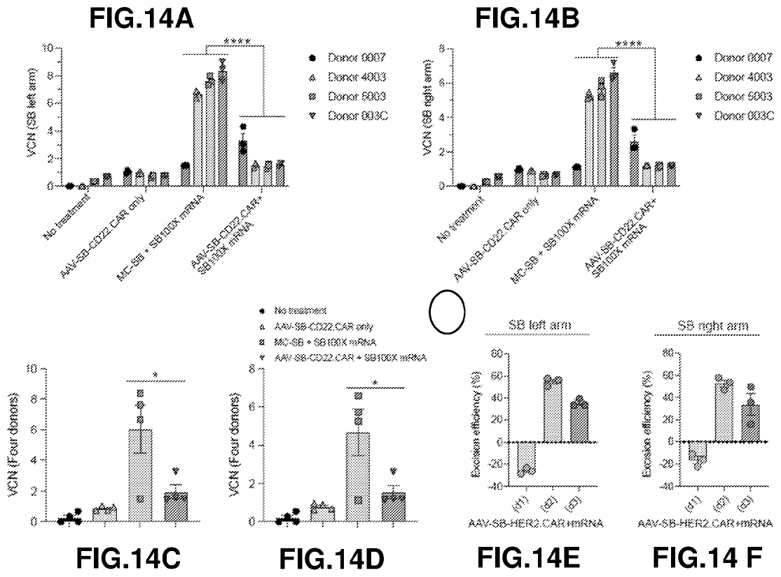

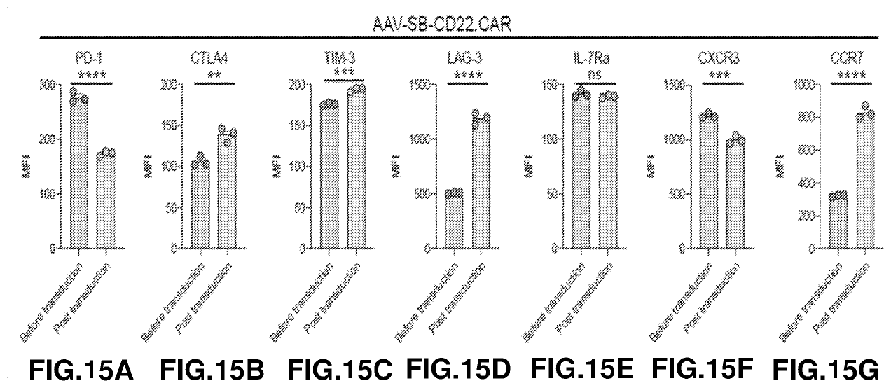

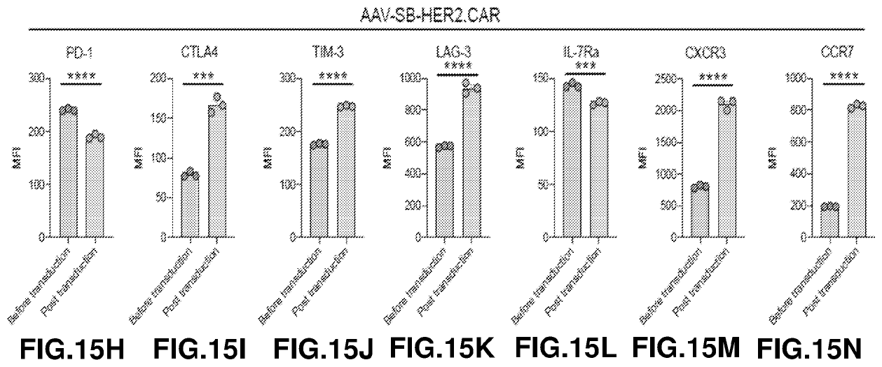

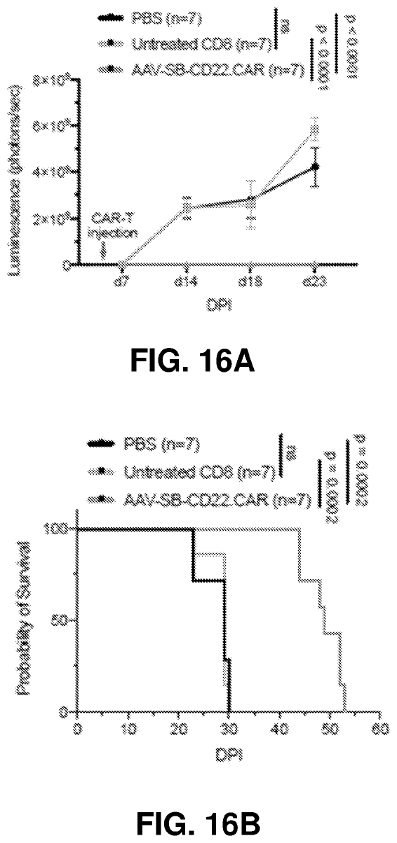

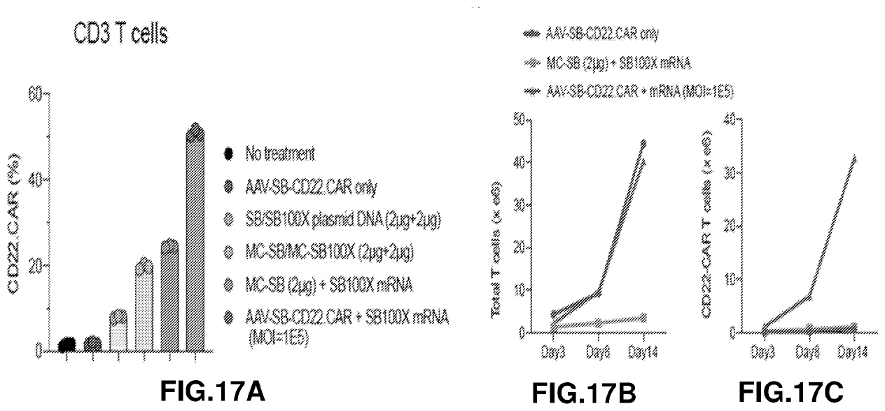

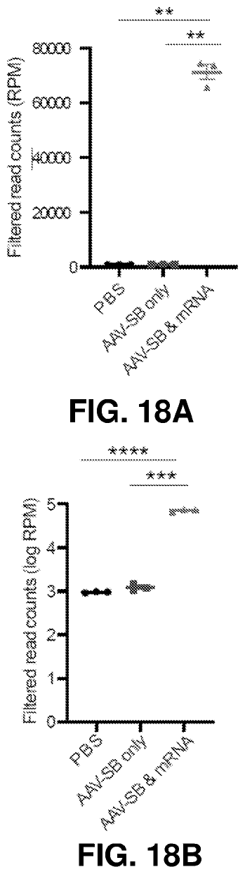

There were two sources for the mRNA used in this study: 1) commercial synthesis by TriLink Biotechnologies (TriLink mRNA was used in FIG. 4C-4F; FIG. 9E-9F; FIG. S2; FIGS. 14A-14E; FIGS. 15A-15N; FIGS. 16A-16B; FIG. 17A-17C and 2) in vitro transcription (used in experiments in figures otherwise) from the SB100x plasmid using the HiScribe T7 ARCA mRNA (with tailing) Kit (NEB). Following RNA transcription, DNase treatment, and poly-A tailing, RNA purification was conducted using the Monarch RNA Cleanup Kit (50 ug) (NEB). After the concentration of the product was measured via Nandrop (with default RNA settings), the RNA was aliquoted and stored in −80° C. RNA was thawed on ice shortly before use in electroporation.

Gene Transfer/CAR Delivery into Human Immune Cells

The vast majority of electroporation experiments were done using a Neon system (ThermoFisher). Before electroporation, cells were collected, washed, and counted. 5×10⁵-3×10⁶ cells were used per reaction, depending on the specific experiment. Per million cells, 1 μg of SB100x mRNA was used. 100 μL Buffer R with the cell and SB100x mRNA mixture was loaded into the Neon Pipette, carefully avoiding the production of bubbles. The electroporation parameter was set at 1600 V, 10 ms, and 3 pulses for T cells, THP-1 cells, and NK-92 cells; 1900 V, 30 ms, and 1 pulse for macrophages. Cells were immediately transferred to a 24-well plate with pre-warmed media after electroporation. Depending on the experiment, specific quantities of AAV were then added to the cells at defined time points vs. electroporation (details in each figure panel and the panel's legend, mostly 0 h if not specified otherwise). Electroporation using Maxcyte system follows a similar procedure except with the manufacturer's suggested electroporation presets. Lentiviral transduction followed standard protocols, mostly following a previous study, with the conditions specified in the figure legends.

### Randomization and Blinding Statements

In vitro cell culture experiments were not randomized. Investigators were not blinded in in vitro cell culture experiments. Mouse experiments were randomized by using littermates, and blinded using generic cage barcodes and eartags where applicable.

### Standard Statistical Analysis

Various standard non-NGS statistical analyses were performed. All statistical methods are described in figure legends and/or supplementary Excel tables. The p values and statistical significance were estimated for data was sourced from different donors. Different levels of statistical significance were accessed based on specific p values and type I error cutoffs (0.05, 0.01, 0.001, 0.0001). Standard analysis was performed using GraphPad Prism. NGS statistics were performed using custom bash and R scripts.

### Code Availability

Scripts used to process the insertion site mapping data will be available on the world wide web at github.com/stanleyzlam/SB-CAR.

### Data, Code and Resource Availability

Data generated or analyzed during this study are included in this published article (and its supplementary information files). Specifically, source data and statistics are provided in an Excel file. Processed read count data for genomic sequencing (e.g. Nextera) is provided in Supplemental Datasets. Raw genome sequence data from the Splinkerette experiments will be deposited to public databases such as NIH Sequence Read Archive (SRA)/Gene Expression Omnibus (GEO) under pending accession number(s). Data and materials that support the findings of this research are available to the academic community from the corresponding author upon reasonable request, via direct sharing, MTAs, or public repositories. Scripts used to process the Splinkerette Nextera samples will be available at internet page github.com/stanleyzlam/SB-CAR.

### Insertion Site Library Preparation and Sequencing

To conduct integration site analysis for the MAJESTIC method, we created a custom protocol to prepare libraries of insertion sites from a CAR-T generation experiment: CD8+ donor 4003 T cells (collected d14 after electroporation). The custom procedure was created by combining elements of protocols from Illumina's NEBNext® Ultra™ II FS DNA Library Prep Kit and, with oligos obtained from the latter. From the non-sorted pool of T cells, roughly 10×10⁶ CAR-Ts were collected. Then, genomic DNA was isolated using the QIAGEN Blood Mini Kit. DNA concentrations were quantified via Nanodrop. 500 ng of genomics DNA were distributed into three separate tubes to serve as three technical replicates for further library preparations. Next, DNA was fragmented for 20 minutes using the Ultra II FS Enzyme Mix from NEB. 100 μM Splinkerette V1.2TS and V1.2BS oligos from IDT were annealed in an Eppendorf tube by heating the mixture to 98 C for 10 min in a heat block and then unplugging the heat block to allow the reaction to cool to room temperature. The final 15 μM annealed Splinkerette adaptor was ligated to the fragmented DNA reactions for 15 mins at 20° C. using NEB Next Ultra II Ligation Master Mix and Ligation Enhancer. Size selection was performed to achieve an insert size distribution of roughly 200-350 bp using NEBNext Sample Purification beads: 30 μL for 1st bead selection 15 μL for 2nd selection.

Then a two-step PCR was performed, using NEBNext Ultra II Q5 Master Mix as the reaction buffer. The first PCR (98° C. 30 s for one cycle; 98° C. 10 s, 65° C. 75 s for 18 cycles; and 65° C. 5 min for one cycle) was used to amplify genomic fragments containing the SB-left arm using two oligos: one specific to the Splinkerette adaptor, and another specific to the SB-left arm. The second PCR (98° C. 30 s for one cycle; 98° C. 10 s, 65° C. 75 s for 12 cycles; and 65° C. 5 min for one cycle) was used to attach i7 index to the library. After each PCR, PCR cleanup using SPRIselect Purification Beads was performed. QC of each key step was performed by running 1 μL of sample on a Tapestation. To quantify each library, aliquots were first diluted 1:10,000. A 1 nM to 0.01 pM dilution series of Illumina PhiX library was then prepared to serve as a standard. The qPCR reaction was prepared using 2× PowerUp SYBR Green, qPCR2.1 and qPCR 2.2 primers at a final concentration of 250 nM, and 5 μL of the diluted libraries in 20 μL total volume. Quantified libraries were diluted to 2 nM and then pooled in equal volumes and denatured according to the Miseq System Denature and Dilute libraries Guide. PhiX was spiked in at 50%, and the denatured pool was diluted to 8 pM and sequenced on Miseq system. A 300-cycles Miseq v2 kit was used to sequence the library with a single-end setting (150 cycles for R1 and 8 cycles for the index1). Two custom sequencing primers (Spl_tag_seq for the index tag and SB_R_pr_seq for the forward read of SleepyBeauty left-end libraries) were spiked into the illumine sequencing primers according to Illumina's bulletin on “Spiking custom primers into the Illumina sequencing primers” (available online at “support.illumina.com/bulletins/2016/04/spiking-custom-primers-into-the-illumina-sequencing-primers-.html”).

### Results

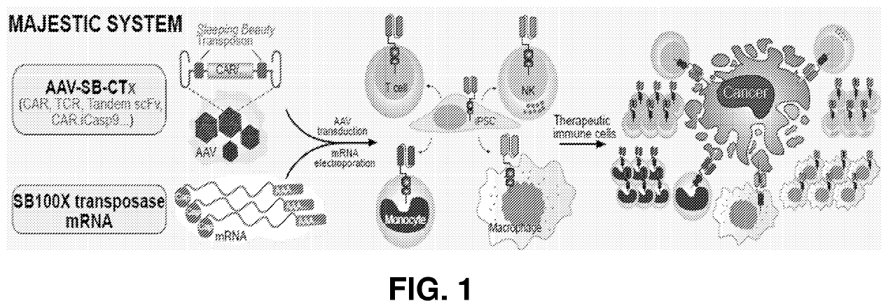

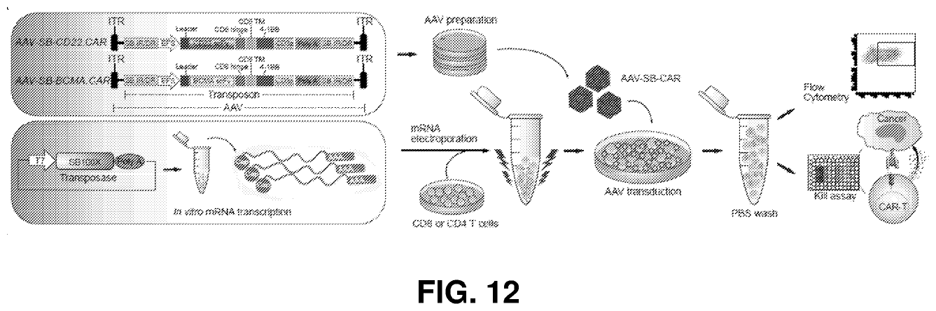

To generate the AAV-SB-CTx plasmid, it was first established the AAV-SB plasmid by cloning the SB transposon, which is flanked by inverted repeats/direct repeats (IR/DR), in between the inverted terminal repeats (ITRs) of the AAV plasmid backbone (Ye, et al. In vivo CRISPR screening in CD8 T cells with AAV-Sleeping Beauty hybrid vectors identifies membrane targets for improving immunotherapy for glioblastoma. Nat Biotechnol 37, 1302-1313, doi:10.1038/s41587-019-0246-4 (2019)). Into this chimeric AAV-SB backbone, single scFv CAR, tandem scFv CAR, TCR, suicide-gene CAR (CAR.iCasp9) constructs were cloned, as depicted the schematics of the hybrid AAV-SB construct, SB100X mRNA electroporation, and CAR-T/NK/macrophage/iPSC generation in FIGS. 1 and 12. The hyperactive SB transposase SB100x (Mates, et al. Molecular evolution of a novel hyperactive Sleeping Beauty transposase enables robust stable gene transfer in vertebrates (Nat Genet 41, 753-761, doi:10.1038/ng.343 (2009)) was delivered to cells via mRNA electroporation to facilitate genomic integration of the SB transposon construct, which was delivered via AAV transduction. Importantly, this two-component design achieves integration of the SB transposon while limiting remobilization compared to that for plasmid transfection because the mRNA encoding the transposase is in principle less stable than plasmid DNA transposase.

To examine the feasibility of this system, CAR-T cell generation was first tested. Human CD4 and CD8 T cells were first electroporated with SB100x mRNA, then transduced them with a titration series of AAV-SB-CD22.CAR virus, as depicted in the schematic of AAV-SB-CD22.CAR, AAV-SB-BCMA.CAR, and SB100X constructs and key procedures of mRNA in vitro transcription, AAV production, mRNA electroporation, flow cytometry, and kill assay in FIG. 12, using multiplicities of infection (MOIs) of 1E3, 1E4, and 1E5. CD22.CAR expression was then monitored via flow cytometry from day 3 to day 14: human CD4 T cells were first electroporated with SB100x mRNA, then transduced with a titration series of AAV-SB-CD22.CAR virus; Human CD8 T cells were first electroporated with SB100x mRNA, then transduced with a titration series of AAV-SB-CD22.CAR virus. Flow cytometry plots of the human CD4 AAV-SB-CD22.CAR T cells and human CD8 AAV-SB-CD22.CAR T were used to monitor CAR-expression levels at various time points from day 3 to day 14.

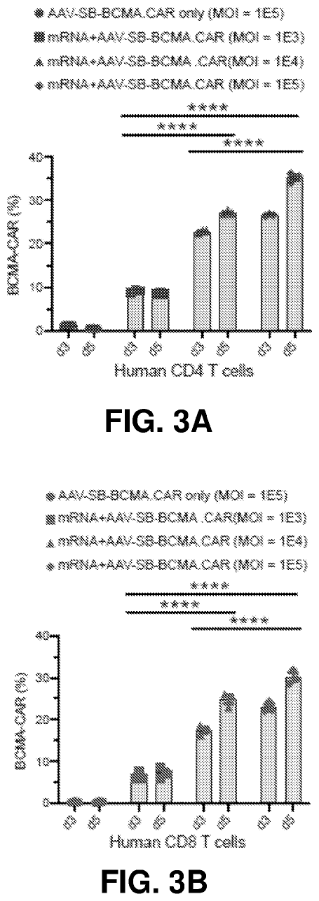

CD22.CAR-positive ratio positively correlated with virus titer, and CAR constructs were stably expressed in all time points tested. At day 14, for MOIs of 1E3, 1E4, and 1E5, respectively, 21.8%, 53.9%, and 60.3% of the total CD4 T cell population were CAR-positive (FIG. 2A), and 17.8%, 39.2%, and 48.7% of CD8 T cells were CAR-positive (FIG. 3A). The experiments were repeated using a different CAR transgene, a BCMA-targeting CAR: at day 5 and at an MOI of 1E5, 35.6% and 32% of CD4 and CD8 T cells were BCMA.CAR-positive, respectively (FIGS. 2B and 3B). Furthermore, T cells in the AAV-SB-CAR condition can be quickly enriched for CAR-positive cells to nearly a pure CAR population following one-time antigen-specific cancer cell stimulation (day 12 post-stimulation), where CD22 CAR+ and BCMA CAR+ T cells were 99.1% and 94.5% of cell populations, respectively, as determined in flow-cytometry of AAV-SB-CD22.CAR and AAV-SB-BCMA.CAR T cells after cancer stimulation. These data serve as proof-of-principle that the MAJESTIC system can efficiently generate stable CAR-T cells.

Next, the optimal time point(s) for AAV-SB-CTx viral transduction relative to SB100x mRNA electroporation were surveyed. Setting SB100x mRNA electroporation as the 0 h time point, cells were transduced with AAV-SB-CTx along a series of time points between −4 h and 4 h. It was observed that the AAV only group showed low CAR expression, although significant over the no-virus control background, potentially due to the transient expression of AAV (FIGS. 4A-4B). In contrast, the MAJESTIC (AAV-SB-CTx) group has substantially higher CAR expression (FIGS. 4A-4B). While the time points have similarly high CAR+ rates, the −4 h time point (i.e., AAV-SB-CTx transduction 4 h before mRNA-SB100x electroporation) appeared to be the most efficient numerically, which was true for both CD4 and CD8 T cells (FIGS. 4A-4B). A timepoint of 0 h-1 h was used for all experiments moving forward, because the efficiency differences between AAV transduction time points are moderate.

The optimal concentration of SB100X transposase was then examined. SB100X mRNA concentration was titrated using a fixed amount of virus (MOI=1E5) and varying amounts of mRNA (FIGS. 4C-4D and 4E-4F). Both CD4 and CD8 T cells electroporated with 1 μg mRNA per 2×10⁶ T cells yielded around 40% CAR-positive T cells. The CAR ratio is higher with 2 μg as compared to 1 g mRNA in both CD4 and CD8 T cells; beyond 2 g of mRNA the CAR ratio appeared to be saturated (FIGS. 4C-4D and 4E-4F). A ratio of 2 g of mRNA per 2×10⁶ cells was used hereafter.

### Example 2: The MAJESTIC System Efficiently Produces CAR-T Cells with High Viability and Yield

CAR-T engineering and generation with the MAJESTIC system was compared with lentiviral vector and SB transposon/transposase plasmid DNA electroporation approaches. These methods are hard to compare as the underlying principles for each method differ. However, head-to-head experiments were performed in parallel to systematically evaluate the performance of each method under standard laboratory settings.

### Methods

### Lentivirus Production and Titration

Low-passage (less than 15 passages) HEK293T cells were used for lentiviral packaging. One day before transfection, 2e7 HEK293T cells were seeded per 150 mm-dish. D10 media was replaced with 13 mL pre-warmed Opti-MEM medium (Invitrogen) before transfection. For each plate, 20 μg transgene plasmid, 15 μg psPAX2 (Addgene), 10 μg pMD2.G (Addgene) and 90 μL lipofectamine 2000 (Thermo Fisher) were mixed in 450 μL Opti-MEM. The mixture was vortexed briefly and incubated for 10-15 min at room temperature, then added dropwise to cells. To minimize the toxicity of lipofectamine, Opti-MEM media was replaced with pre-warmed 20 mL D10 media 5-6 h after transfection. Viral supernatant was collected 48 h post-transfection and then concentrated using the Amicon Ultra-15 Centrifugal filter unit (Millipore) or purified with Lenti-X Concentrator (Takara). All virus was titrated with Lenti-X GoStix Plus (Takara) before being aliquoted and stored in −80° C.

### Kill Assay

To interrogate AAV-SB-CAR T cell killing efficacy, NALM6-GL (GFP-Luciferase), MM.1R-GL, and MCF7-PL (Puromycin-Luciferase) cancer cell lines were seeded in 96-well plates. Corresponding CAR-T or CAR-NK cells were then added according to various effector to target (T/NK cell:cancer cell) ratios. Cytolysis was measured through luciferase assays. 150 μg/mL D-Luciferin (PerkinElmer) was added to the plate using a multi-channel pipette. Following a 5 minutes incubation at room temperature, luciferin intensity was measured by a Plate Reader (PerkinElmer).

### Results

The functional multiplicity of infection (MOI) for AAV (in transduction units) is usually 3-4 orders of magnitude lower than that of genomic MOI (in genome copies; GCs, gcs) (Francois, et al. Accurate Titration of Infectious AAV Particles Requires Measurement of Biologically Active Vector Genomes and Suitable Controls. Mol Ther Methods Clin Dev 10, 223-236, doi:10.1016/j.omtm.2018.07.004 (2018)); therefore, AAV genomic MOIs between 1E4-5E5, and a lentiviral MOIs between 1-10 were used in these experiments. For DNA plasmid electroporation, between 1-4 g total DNA/1e6 cells was used, which is similar to the range of concentrations tested previously (Chicaybam, et al. Transposon-mediated generation of CAR-T cells shows efficient anti B-cell leukemia response after ex vivo expansion. Gene Ther 27, 85-95, doi:10.1038/s41434-020-0121-4 (2020)).

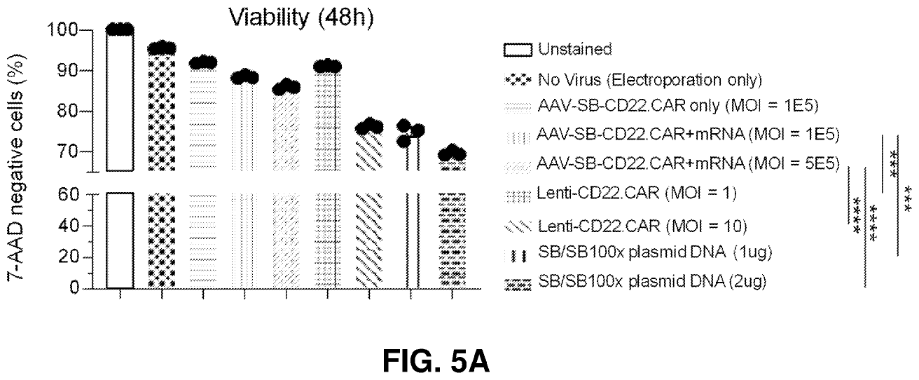

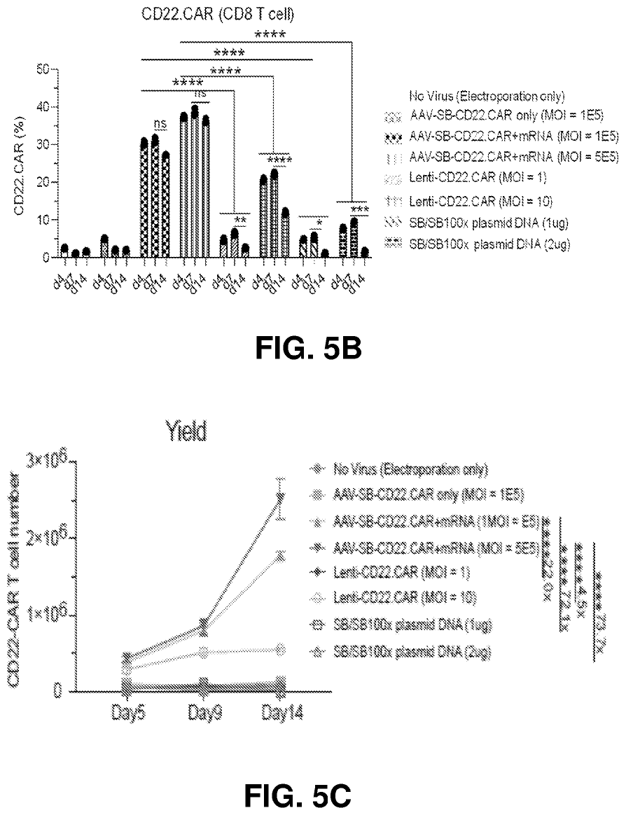

After CAR-T generation with all three platforms (MAJESTIC, lentivirus and SB transposon plasmid electroporation), cell viability was first determined via 7-AAD staining 48 h after electroporation. Flow cytometry results measuring viability of CD8 T cells after mRNA electroporation, plasmid DNA electroporation, and lentivirus transduction showed that the MAJESTIC (AAV-SB-CD22.CAR+SB100x-mRNA) groups had viability above 85%. Lenti-CD22.CAR cells were over 90% viable at an MOI of 1 but only about 75% viable at a higher MOI of 10 (FIG. 5A). Cells electroporated with plasmid DNA demonstrated lower viability: 75% and 70% for 1 μg and 2 g of plasmid DNA, respectively (FIG. 5A). Under these conditions, flow cytometry plots of human CD8 T cells transduced with AAV-SB-CD22.CAR virus (MOI=1E5 and 5E5), transduced with CD22.CAR lentivirus (MOI=1 and 10), or electroporated with plasmid DNA (1 μg=0.5 g transposon plasmid+0.5 g transposase plasmid) at three time points were used to quantify cells. On day 4, the flow cytometry data revealed that Lenti-CD22.CAR at a high MOI (MOI=10) yielded 21% CD22.CAR-positive CD8 T cells and only 4.32% at a low MOI (MOI=1). Even lower CAR-positive population percentages were observed for plasmid DNA electroporation groups (4.56% at 1 g and 7.96% at 2 μg) compared to lentivirus. Substantially higher CAR expression was observed in the MAJESTIC groups, with 30.1% (MOI=1E5) and 37.1% (MOI=5E5) CD22.CAR-positive T cells, the stability of CAR-positive T cell populations was monitored by examining CD22.CAR T populations at day 7 and day 14 by flow cytometry. CAR-positive populations were largely stable in the MAJESTIC groups, but significantly declined in the lentivirus and plasmid groups (FIG. 5A).

Another experiment was performed to specifically analyze the phenomenon that Lenti-CAR % declined across time in culture. After viral transduction and/or electroporation, Lenti-CAR and MAJESTIC-CAR T cells were sorted on day 2. Both normal Lenti-CD22.CAR and spin-infected Lenti-CD22. CAR groups demonstrated reduction of CAR+ percentages by day 5 (68.3% and 61.3%), falling even further by day 13 (52% and 43%). MAJESTIC-CAR T cells maintained a stable CAR+ ratio of around 85% (89.3% right after sorting). Although it is not entirely certain why lentiviral CAR ratios decline, it has been known that lentivirus transgenes can often get silenced, potentially leading to reduced CAR percentages. CAR-T cell yield is an important joint outcome of viability, efficiency and proliferation (yield=CAR positive percentage*total live cell count), all of which can be affected by the cell states post CAR transgene delivery. The yield was estimated on day 5, 9, and 14. As a result, starting from approximately equal quantities of human CD8 T cells, the yields of CAR+ T cells from the MAJESTIC groups were much higher than those of lentivirus and of plasmid electroporation (e.g. 4.5× and 73.7× higher at high dose, respectively) (FIGS. 5B/5C). These data suggested that, in the laboratory settings specified, that the MAJESTIC system is much more efficient in generating viable and stable CAR-T cells than lentiviral or DNA transposon electroporation approaches. Moreover, MAJESTIC was also tested using the Maxcyte electroporation platform: applying the MAJESTIC method using both the Neon and Maxcyte electroporators for introduction of the SB transposase (SB100x) mRNA component into cells yielded CAR percentages of over 36%, suggesting that MAJESTIC is not limited to one electroporation platform.

### Example 3: Initial Characterization of CAR-T Cells Generated by MAJESTIC

### Methods

### Copy Number Determination

T cells were collected at d21 after electroporation and washed with PBS twice to remove media. Cells were incubated with CD22-Fc protein (R&D Systems) in PBS for 30 min on ice and washed with PBS twice to remove the unbonded protein. The cells were then stained with anti-human IgG Fc-APC (Biolegend, Cat #366906) on ice for 30 min and washed with PBS twice. The CAR-positive T cells were purified by Anti-APC MicroBeads (Miltenyi). After that, genomic DNA was extracted using the QIAGEN Blood Mini Kit. To minimize episomal AAV contamination, the extracted genomic DNA samples were separated on a 1% agarose gel, and DNA bands over 10 kbp were gel isolated and purified with the QIAquick Gel Extraction Kit (QIAGEN). qPCR was conducted to determine copy number, using primers that specifically target the SB transposon:

IRDR-left, “Forward” from 5′-3′:
(SEQ ID NO: 44)
CTCGTTTTTCAACTACTCCACAAATTTCT.
IRDR-left, “Reverse” from 5′-3′:
(SEQ ID NO: 45)
GTGTCATGCACAAAGTAGATGTCCTA.
IRDR-right, “Forward” from 5′-3′:
(SEQ ID NO: 46)
GCTGAAATGAATCATTCTCTCTACTATTATTCTGA.
IRDR-right, “Reverse” from 5′-3′:
(SEQ ID NO: 47)
AATTCCCTGTCTTAGGTCAGTTAGGA.

As an internal control we amplified samples with primers for RPPH1, a housekeeping gene known to have two copies per cell:

“Forward” from 5′-3′:
(SEQ ID NO: 48)
AGCTGAGTGCGTCCTGTCACT.
“Reverse” from 5′-3′:
(SEQ ID NO: 49)
TCTGGCCCTAGTCTCAGACCTT.
PCR 1 primer on transposon end
(“SB_R_pr_1”) from 5′-3′:
(SEQ ID NO: 50)
T*TTGTTAACAAGAAATTTGTGGAGTAGTT*G.
PCR 2 primer on transposon end
(“SB_R_pr_2”) from 5′-3′:
(SEQ ID NO: 51)
A*ATGATACGGCGACCACCGAGATCTACACAAAAACGAGTTTTAATG
ACTCCAA*C.
qPCR primer for library quantification
(“qPCR2.1”) from 5′-3′:
(SEQ ID NO: 52)
A*ATGATACGGCGACCACCGAGAT*C.
qPCR primer for library quantification
(“qPCR2.2”) from 5′-3′:
(SEQ ID NO: 53)
C*AAGCAGAAGACGGCATACGAGA*T.
Miseq reverse primer (“Spl_rev_seq”) from 5′-3′:
(SEQ ID NO: 54)
T*AATACGACTCACTATAGGTGACAGCGAGCGC*T.
Miseq index primer (“Spl_tag_seq”) from 5′-3′:
(SEQ ID NO: 55)
A*GCGCTCGCTGTCACCTATAGTGAGTCGTATT*A.
Miseq read1 primer (“SB_R_pr_seq”) from 5′-3′:
(SEQ ID NO: 56)
A*AAAACGAGTTTTAATGACTCCAACTTAAGTGTATGTAAACTTCC*G
Splinkerette adaptor (anneal with V1.2TS)
“Splinkerette V1.2BS” from 5′-3′:
(SEQ ID NO: 57)
G*CGCTCGCTGTCACCTATAGTGAGTCGTATTATAATTTTTTTTTCAA
AAAA*A.
Splinkerette adaptor (anneal with V1.2BS)
“SplinkeretteV1.2TS” from 5′-3′:
(SEQ ID NO: 58)
G*TTCCCATGGTACTACTCATATAATACGACTCACTATAGGTGACAGC
GAGCGC*T.
PCR 1 on Splink end “SplAP1” from 5′-3′:
(SEQ ID NO: 59)
G*TTCCCATGGTACTACTCAT*A.
PCR 2 on Splink end, adds sequencing index
“SplAP2_V1.1” from 5′-3′:
(SEQ ID NO: 60)
C*AAGCAGAAGACGGCATACGAGATCGGTACAAGCTATAATACGACT
CACTATAG*G.
PCR 2 on Splink end, adds sequencing index
“SplAP2_V1.2” from 5′-3′:
(SEQ ID NO: 61)
C*AAGCAGAAGACGGCATACGAGATCGGTAAACATCGTAATACGACT
CACTATAG*G.
PCR 2 on Splink end, adds sequencing index
“SplAP2_V1.3” from 5′-3′:
(SEQ ID NO: 62)
C*AAGCAGAAGACGGCATACGAGATCGGTACATTGGCTAATACGACT
CACTATAG*G.
PCR 2 on Splink end, adds sequencing index
“SplAP2_V1.4” from 5′-3′:
(SEQ ID NO: 63)
C*AAGCAGAAGACGGCATACGAGATCGGTACCACTGTTAATACGACT
CACTATAG*G.
PCR 2 on Splink end, adds sequencing index
“SplAP2_V1.5” from 5′-3′:
(SEQ ID NO: 64)
C*AAGCAGAAGACGGCATACGAGATCGGTAACGTGATTAATACGACT
CACTATAG*G.
PCR 2 on Splink end, adds sequencing index
“SplAP2_V1.6” from 5′-3′:
(SEQ ID NO: 65)
C*AAGCAGAAGACGGCATACGAGATCGGTCGCTGATCTAATACGACT
CACTATAG*G.
PCR 2 on Splink end, adds sequencing index
“SplAP2_V1.7” from 5′-3′:
(SEQ ID NO: 66)
C*AAGCAGAAGACGGCATACGAGATCGGTCAGATCTGTAATACGACT
CACTATAG*G.
PCR 2 on Splink end, adds sequencing index
“SplAP2_V1.8” from 5′-3′:
(SEQ ID NO: 67)
C*AAGCAGAAGACGGCATACGAGATCGGTATGCCTAATAATACGACT
CACTATAG*G.
PCR 2 on Splink end, adds sequencing index
“SplAP2_V1.9” from 5′-3′:
(SEQ ID NO: 68)
C*AAGCAGAAGACGGCATACGAGATCGGTCTGTAGCCTAATACGACT
CACTATAG*G.
PCR 2 on Splink end, adds sequencing index
“SplAP2_V1.10” from 5′-3′:
(SEQ ID NO: 69)
C*AAGCAGAAGACGGCATACGAGATCGGTAGTACAAGTAATACGACT
CACTATAG*G.
PCR 2 on Splink end, adds sequencing index
“SplAP2_V1.11” from 5′-3′:
(SEQ ID NO: 70)
C*AAGCAGAAGACGGCATACGAGATCGGTCATCAAGTTAATACGACT
CACTATAG*G.
PCR 2 on Splink end, adds sequencing index
“SplAP2_V1.12” from 5′-3′:
(SEQ ID NO: 71)
C*AAGCAGAAGACGGCATACGAGATCGGTAGTGGTCATAATACGACT
CACTATAG*G.
PCR 2 on Splink end, adds sequencing index
“SplAP2_V1.13” from 5′-3′:
(SEQ ID NO: 72)
C*AAGCAGAAGACGGCATACGAGATCGGTAACAACCATAATACGACT
CACTATAG*G.
PCR 2 on Splink end, adds sequencing index
“SplAP2_V1.14” from 5′-3′:
(SEQ ID NO: 73)
C*AAGCAGAAGACGGCATACGAGATCGGTAACCGAGATAATACGACT
CACTATAG*G.
PCR 2 on Splink end, adds sequencing index
“SplAP2_V1.15” from 5′-3′:
(SEQ ID NO: 74)
C*AAGCAGAAGACGGCATACGAGATCGGTAACGCTTATAATACGACT
CACTATAG*G.
PCR 2 on Splink end, adds sequencing index
“SplAP2_V1.16” from 5′-3′:
(SEQ ID NO: 75)
C*AAGCAGAAGACGGCATACGAGATCGGTAAGACGGATAATACGACT
CACTATAG*G.
PCR 2 on Splink end, adds sequencing index
“SplAP2_V1.17” from 5′-3′:
(SEQ ID NO: 76)
C*AAGCAGAAGACGGCATACGAGATCGGTAAGGTACATAATACGACT
CACTATAG*G.
PCR 2 on Splink end, adds sequencing index
“SplAP2_V1.18” from 5′-3′:
(SEQ ID NO: 77)
C*AAGCAGAAGACGGCATACGAGATCGGTACACAGAATAATACGACT
CACTATAG*G.

The qPCR reactions were set up with 30 ng of genomic DNA (using three technical replicates), forward and reverse primers at a final concentration of 250 nM, and SYBR Green PowerUp Master Mix (ThermoFisher). Reactions were run in standard mode: a 2 min hold at 95° C. followed by 40 cycles of 15 s at 95° C. to denature and 60 s at 60° C. to anneal and extend.

### Excision Efficiency Determination

T cells were harvested at different time points after SB100x mRNA and viral transduction for transposase excision efficiency evaluation. Primers 5′-3′: CCGCACGCGTTCTAGACT (SEQ ID NO:20) targeting AAV backbone and 5′-3′: ACAAAGTAGATGTCCTAACTGACTTGCC (SEQ ID NO:21) targeting SB left arm were designed to evaluate SB left arm excision efficiency. Primers are 5′-3′: GCCGCTCGGTCCGCACGTG (SEQ ID NO:22) targeting AAV backbone and 5′-3′: AGTGAGTTTAAATGTATTTGGCTAAGGTGTATG (SEQ ID NO:23) targeting SB right arm were designed to evaluate SB right arm excision efficiency. The SYBR Green master Mix (ThermoFisher) was applied for qPCR quantification as previously described. For the excision efficiency calculation, AAV-SB-CAR only group (only transduced with AAV) was determined as baseline level of viral copy number that was existed in the T cells, then viral copy number in AAV-SB-CAR+SB100x mRNA group was divided by the baseline viral copy number, which was determined as excision efficiency.

### In Vivo CAR-T Cell Functionality Testing

NSG mice were intravenously injected with 5×10⁵ NALM6-GL cancer cells. After four days of cancer inoculation, 5×10⁶ CD22.CAR T cells were tail vein injected as treatments. Bioluminescent imaging was performed via IVIS system to monitor leukemia progression. Animal survival study followed an approved death-as-endpoint protocol.

### Results

To estimate the vector copy number (VCN) of CAR transgenes per cell in CAR-T cells generated by MAJESTIC, a later time point (day 21) was chosen with the aim to measure stable transgenes. Using a standard approach similar to others in the field ((Kolacsek, et al. Mob DNA 2, 5 (2011)), the VCN of MAJESTIC- and MC-SB+SB100X mRNA-generated CD22 CAR-T cells in four different human donors was estimated. The data showed that, under this experimental condition, the AAV-only group shows an average VCN of approximately 1. This detected background level may be due to prolonged episomal persistence of the AAV vector, and/or random genomic integration of the AAV vector DNA. MC-SB+SB100X mRNA group had a VCN of approximately 1-9 copies/cell. MAJESTIC group was observed with VCNs of approximately 1-4 copies/cell (FIG. 14A-14D). VCN measurement using both left arm and right arm probes showed consistent results (FIG. 14A-14D) excision circles were also quantified as a proxy for the excision efficiency of the SB100X transposase. AAV-SB-CAR constructs were significantly processed after one day of SB100X mRNA electroporation. qPCR primers amplifying the junction between the AAV arms and SB arms were used, for both the left and right sides separately to verify the assay.

To address whether the CAR-T cells generated by MAJESTIC were functional, cancer killing assays were performed by co-culturing cancer cells and CAR-T cells, generated either via MAJESTIC or lentivirus.

Two CAR-T: cancer models were evaluated in co-culture, including CD22.CAR vs. NALM6-GL cancer cells and BCMA.CAR vs. MM.1R-GL cancer cells: Cytolysis analysis of NAML6-GL (NAML6 with GFP and luciferase reporters) cancer cells that were co-cultured with Lenti-CD22.CAR and AAV-SB-CD22.CAR T cells was carried out with CAR-Ts seeded at various effector:target (E:T) ratios, and luciferase imaging was performed at two time points (16 h and 40 h); likewise, cytolysis analysis of MM.1R-GL (MM.1R with GFP and luciferase reporters) cancer cells that were co-cultured with Lenti-BCMA.CAR and AAV-SB-BCMA.CAR T cells was carried out with CAR-Ts seeded at various effector:target (E:T) ratios, and luciferase imaging was performed at two time points (16 h and 40 h). The DNA plasmid electroporation group was excluded because those cells did not expand well and the yields were too low to be practically useful.

In amplifying the left arm, excision efficiency was around 55% on day 2 and 36% on day 3 (FIGS. 14E-4F). With the right arm, excision efficiency was consistent, at around 53% on day 2, and 34% on day 3 (FIGS. 14E-4F). Of note, the excision efficiency was slightly higher on day 2 compared to day 3, which may be due to the degradation of SB100X mRNA. This aligns with the goal of using mRNA in the MAJESTIC system to avoid prolong expression or existence of the transposase to minimize unnecessary transposon jumping or excision after transgene delivery.

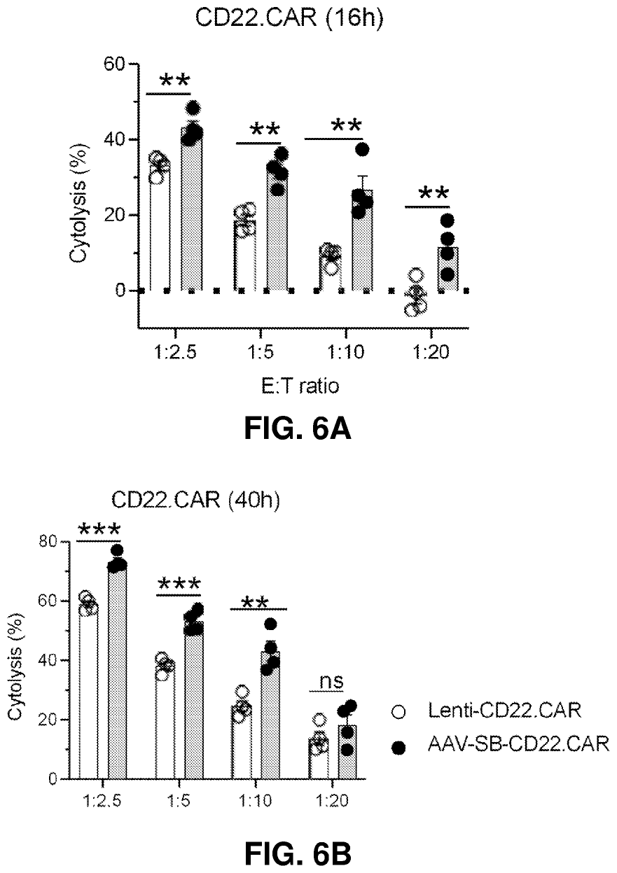

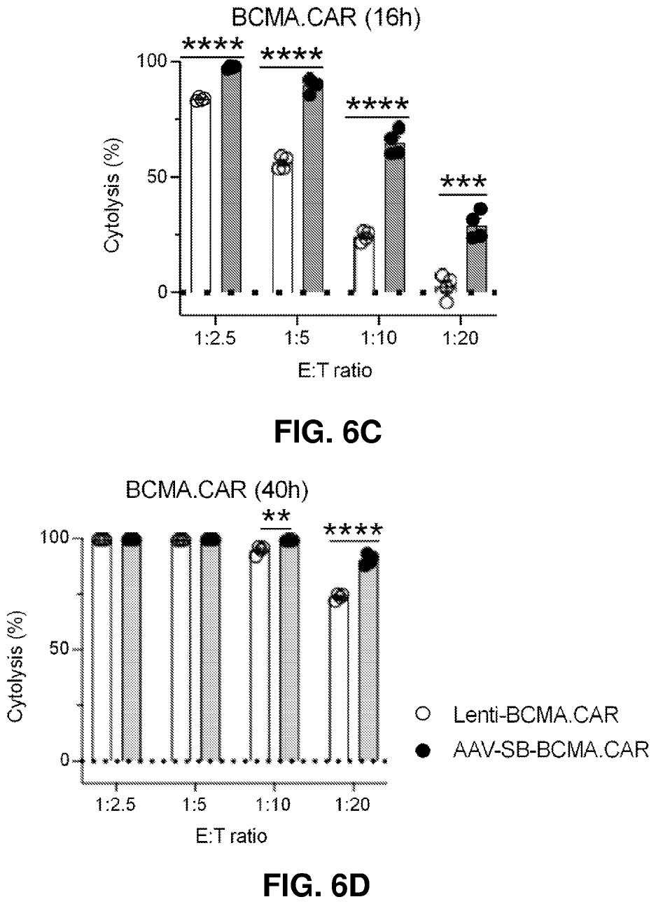

In counting T cells for kill assays, the number of T cells applied by CAR T ratio was normalized to ensure each group received the same number of CAR-T cells. While both MAJESTIC-generated CAR-T cells (AAV-SB-CAR) and lentiviral-transduced CAR-T cells (Lenti-CAR) exhibited tumor cell killing across the board, both AAV-SB-CD22.CAR and AAV-SB-BCMA.CAR T cells manifested significantly stronger killing over their lentivirus-mediated counterparts, for matched Effector:Target (E:T) ratios (FIGS. 6A-6D). These data demonstrate that the CAR-T cells generated by MAJESTIC were indeed functional, with potential advantage over the lentiviral CAR system in the settings tested.

The surface markers of MAJESTIC-generated CD8 CAR-T cells were then further characterized and whether virus and mRNA introduction would affect the immune phenotypes of T cells was explored. T cell exhaustion and memory markers before and after electroporation and viral transduction were evaluated by staining for CD22.CAR and HER2.CAR T cells. The flow cytometry data showed that PD-1 was slightly decreased post MAJESTIC; while CTLA-4, TIM-3, and LAG-3 were increased; nevertheless, PD-1, CLTA-4, and TIM-3 all remained at baseline level as measured by mean fluorescence intensity (MFI) (FIGS. 15A-15N). For the memory markers, only CCR7 demonstrated consistent increase in both HER2.CAR and CD22.CAR T cells. IL-7Ra remained at baseline for CD22.CAR and decreased for HER2.CAR (FIGS. 15A-15N). CXCR3 demonstrated differing expression patterns, potentially due to differences in the CAR constructs (e.g., regarding costimulatory domains, CD22.CAR has 4-1BB, while HER2.CAR has 4-1BB and CD28). To further assess whether the CAR-T cells generated by MAJESTIC were effective against cancer, a CAR-T efficacy testing experiment was performed in vivo using an animal model of B cell leukemia with adoptive cell transfer treatment. This in vivo experiment was intended only to validate that MAJESTIC-generated CAR-T were indeed functional, but not to compare with cells generated by other platforms. The results showed strong anti-tumor efficacy, where MAJESTIC produced CAR-T cells, but not unmodified CD8 T cells, substantially suppressed cancer progression as measured by IVIS-bioluminescence imaging (p<0.0001) (FIG. 16A), and significantly extended the overall survival of treated mice (p=0.0002) (FIG. 16B). The animals in this cohort were solely used for IVIS imaging and survival analysis to demonstrate that MAJESTIC-produced CAR-T cells are functional; thus, they were not euthanized concurrently. In the future, it may be feasible and informative to use MAJESTIC to generate CAR-T cells and to evaluate their phenotypes (e.g., persistence potential) in vivo. Together, these data showed that the MAJESTIC system can efficiently produce stable and functional CAR-T cells with high viability and yield.

### Example 4: Application of MAJESTIC in an Array of Different Types of Therapeutic Transgenes in Human T Cells

### Methods

### Splinkerette PCR

Splinkerette PCR was performed to amplify integration sites of the Sleeping Beauty cassette. Fourteen days after SB100x mRNA electroporation and AAV transduction of human NK-92 cells, cells were harvested and genomic DNA was extracted using the QIamp Blood Mini Kit. Sau3AI (NEB) was used to digest 1 g genomic DNA for 4 h at 37° C., which was followed by 65° C. heat inactivation for 20 min. Forward and reverse Splinkerette adaptors were mixed to a final concentration of 25 μM and annealed as follows: denaturing 95° C. for 5 min and then cooling to room temperature at a ramp down rate of 5° C./min. Shortly thereafter, annealed adaptors (25 μM) were ligated overnight to Sau3AI digested genomic DNA using T4 Ligase (NEB). To amplify out inserted transposon arms and the flanking genomic DNA sequences, a nested PCR was performed with Splink 1 and SB-Left 1 primers for the 1^{st} PCR and Splink 2 and SB-Left 2 primers for the 2^{nd} PCR. The products were run on Invitrogen 2% E-Gels, and bands roughly within the 100-700 bp range were excised. Gel purification was performed with the QIAGEN kit and the products were stored at −20° C.

### Splinkerette Library Preparation and Nextera Sequencing

Preparation of the sequencing library was performed using the Nextera XT Library Prep Kit. Briefly, gel-purified Splinkerette PCR products were diluted to 0.4 ng/L, separated into three technical replicates, and subjected to tagmentation at 55° C. for 8 min. Then, tagmented DNA was amplified and Nextera index adaptors (N701-706 and S506-508) were added via a 13-cycle PCR. Then, a TapeStation 4150 was used to measure the concentration of bands within the 100-700 bp range for each of the PCR products to normalize samples for pooling. Pooled samples were purified using the QIAGEN PCR Cleanup Kit and stored at −20° C. Samples were prepared for sequencing via the Illumina Miseq System following manufacturer's instructions. Specifically, the library was diluted to under 10 pM and spiked with 5% PhiX Control, and the 150-cycle MiSeq Reagent Kit was used.

### Splinkerette Data Processing and Analysis, and Visualization

Single end FASTQ reads were quality trimmed with BBDuk 79 using the settings trimq=27 minlen=80 maq=30 qtrim=rl. Then, non-integrated AAV-SB sequences were removed by using a sequence specific to the AAV ITR and by using Cutadapt 80 with the following settings: -g TATAGTCTAGAACGCGTGCG (SEQ ID NO:24) -e 0.1 --overlap 15 --discard-trimmed. Then, to trim out the transposon arms and keep only the sequences that were trimmed, we used Cutadapt with the settings -g \hat{ }ACTTCAACTG (SEQ ID NO:78) -e 0.1 -m 15 --overlap 10 --discard-untrimmed. Ten bases were removed from the 5′ end (cutadapt -u 10) and the all reads were trimmed to a fixed length of 30 (cutadapt -l 30). Reads were mapped using hisat2 5981 onto the HISAT2 indexed GRCh38 genome.

Samtools view was used to filter out mapped reads with a quality score of less than 30, and the files were subsequently converted to the BED format using samtools view (Li, et al. Bioinformatics 25, 2078-2079 (2009), and bedtools bamtobed (Quinlan, & Hall, Bioinformatics 26, 841-842, (2010)). Genomic coordinate files in .bed format were loaded into R, keeping only the starting genomic coordinate. They were further processed and formatted into GRanges objects for data visualization. Key packages used for R processing and visualization include GenomicRanges (Lawrence, et al. PLoS Comput Biol 9, e1003118 (2013), genomation (Akalin, et al., Bioinformatics 31, 1127-1129 (2015), ggbio (Yin, et al., Genome Biol 13, R77 (2012), BRGenomics, and pheatmap.

### Sample Size Determination

Sample size was determined according to the lab's prior work, cited literature, or similar approaches in the field.

### Replication

Experimental replications are indicated in each figure panel's legend. Important experiments have been repeated independently to ensure reproducibility.

### Data Collection Summary

Flow cytometry data was collected by BD FACSAria. Co-culture killing assay data were collected with Perkin Elmer Envision Plate Reader.

### Data Analysis Summary

Flow cytometry data were analyzed by FlowJo v.10.7. All simple statistical analyses were done with Prism 9. All NGS analyses were performed using custom bash and R scripts.

### Results

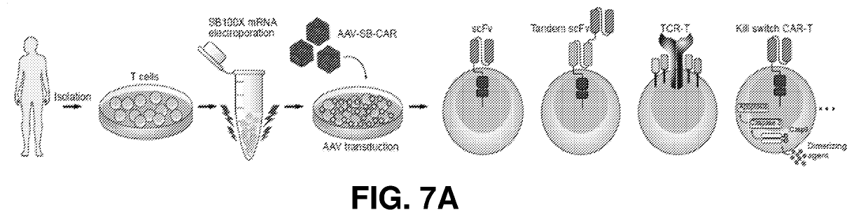

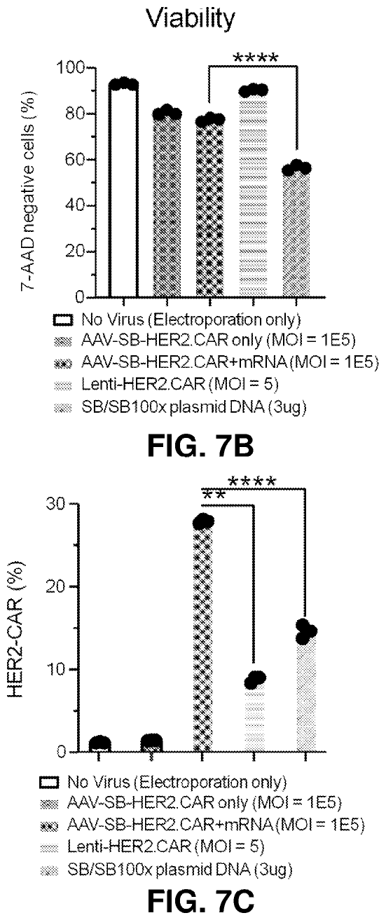

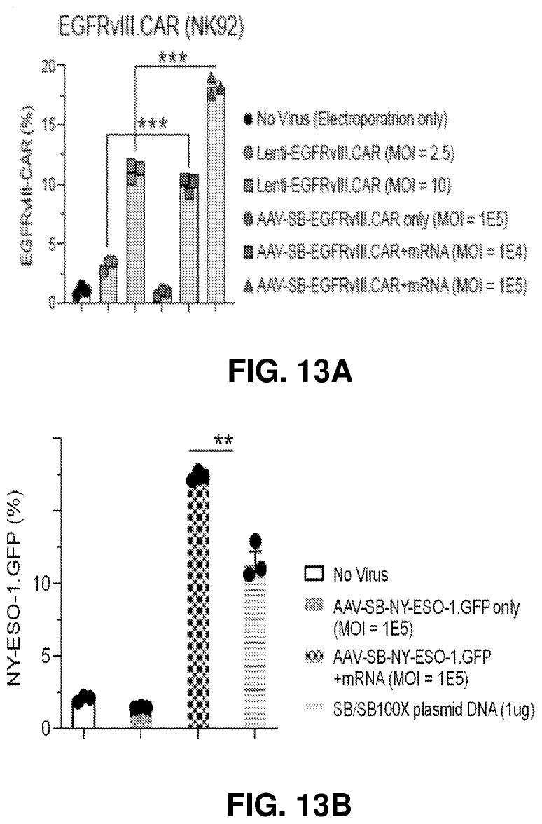

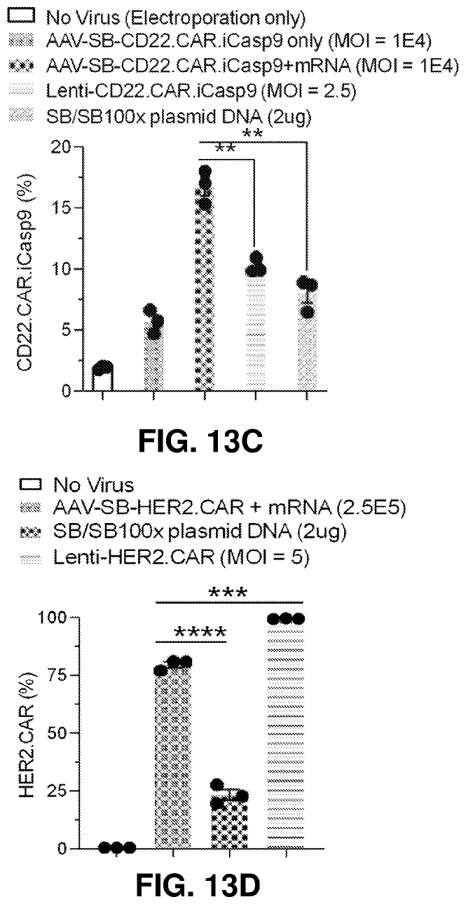

This technology was next applied for engineering other types of therapeutic transgenes in human primary T cells, such as solid tumor CAR-T, tandem scFv (bispecific) CAR-T, TCR-T, and suicide-gene CAR-T cells, as depicted in FIG. 7A. For solid-tumor-specific CARs, HER2-specific CAR-T cells were generated, again using MAJESTIC, lentivirus, and plasmid DNA electroporation gene-transfer methods (FIG. 7B). MAJESTIC produced the highest percentage of HER2.CAR-positive cells (28.1%), as determined by flow-cytometry with 7-AAD staining (FIG. 7C). Lenti-HER2.CAR and plasmid DNA groups were less efficient, at 8.99% and 13.7%, respectively. EGFRvIII-specific CAR-T cells were also generated in a similar manner. Although efficiency was generally lower, again, MAJESTIC's CAR % T cell production efficiency was significantly higher than those of lentivirus (FIG. 13A).

Bispecific CAR-Ts can recognize two antigens and may thereby reduce the chance of immune escape; such systems have demonstrated potent efficacy against relapsed B cell malignancies that down-regulated single target antigen expression (Kustikova, et al. Clonal dominance of hematopoietic stem cells triggered by retroviral gene marking. Science 308, 1171-1174, doi:10.1126/science.1105063 (2005)).

To establish that MAJESTIC can be used for bi-specific CAR-T generation, an anti-CD19/anti-CD20 tandem scFv construct was designed (AAV-SB-CD19.20.CAR construct) according to the Schematic representation in FIG. 8A. The construct includes the CD19 scFv and CD20 scFv CAR sequences joined by a linker to be expressed together as a tandem scFv CAR. This construct was used to transduce primary human T cells, again comparing MAJESTIC to lentivirus and DNA transposon systems in parallel. Flow cytometry was performed on days 5 and 10, after electroporation and viral transduction. As with single CARs, the efficiency of MAJESTIC was significantly higher than that of lentivirus, under the conditions tested (FIG. 8B). Again, the CAR+% of the bispecific CAR-T cells was stable in the MAJESTIC group, but was significantly reduced for both the lentivirus and DNA transposon systems (FIG. 8C). In evaluating antigen expression of the cognate leukemia cancer cells, (cytolysis analysis of NALM6-GL cancer cells that were co-cultured with lenti-CD19.20.CAR and AAV-SB-CD19.20.CAR T cells), high CD19 expression, but weak CD20 bi-specific CAR-T cells exhibited strong killing ability, with nearly 100% and around 80% cytolysis after 17 h at E:T ratios of 1:4 and 1:10, respectively, for both MAJESTIC and lentiviral CAR-T cells (FIG. 8D). These data suggested that MAJESTIC can efficiently deliver a bispecific CAR construct into human T cells.

To test the utility of MAJESTIC for TCR-T cell production, an NY-ESO-1 TCR construct was cloned along with a GFP marker into the AAV-SB backbone. Flow cytometry plots of NY-ESO-1 T cells indicated that AAV-SB-NY-ESO-1.GFP transduction plus SB100x mRNA electroporation was able to generate some fraction of NY-ESO-1 TCR-T cells, which was still significantly higher than that using SB/SB100x plasmid DNA electroporation (17% vs 11%) (FIGS. 13A-13B). Conditional inactivation of CAR-T cells is important to control potential toxicity, using kill-switch elements such as induced Caspase 9 (iCasp9) (Straathof, et al. An inducible caspase 9 safety switch for T-cell therapy. Blood 105, 4247-4254, doi:10.1182/blood-2004-11-4564 (2005)) conditional control CAR-T cells were generated with two transgenes, CD22 CAR and a suicide-gene (CD22.CAR.iCasp9 T cells), as depicted in the chematic representation of the AAV-SB-CD19.20.CAR construct in FIG. 9A (CD19 scFv and CD20 scFv CAR sequences are joined by a linker and are expressed together as a tandem scFv CAR).

Flow cytometry data showed that the efficiency of the MAJESTIC system was higher than that of lentiviral and plasmid systems (FIGS. 9B and 13C). The CAR-positive T cells were further enriched in AAV-SB-CD22.CAR.iCasp9+mRNA group (53.5%) at day 5 after antigen-specific cancer cells stimulation (FIG. 9C). Altogether, these data demonstrated the versatility of the MAJESTIC system for delivering a variety of payloads to generate various therapeutic T cells.

### Example 5: Head-to-Head Comparison of MAJESTIC System with Mini-Circle DNA System

### Methods

### Mouse Model

Mice were housed in standard conditions in Yale vivarium, maintained on a 12 h light/dark cycle (07:00 to 19:00 light on). Mice, both female and male, aged 8-12 weeks were used for experiments. NOD-scid TL2Rgammanull (NSG) mice were purchased from JAX and bred in-house for T cell-based anti-tumor therapeutic efficacy testing experiments. Mouse health was monitored daily after tumor induction.

### Preparation of Minicircle (MC) DNA

Genes-of-interest such as SB-CAR and SB100X constructs were firstly cloned into a parental plasmid (System Biosciences). Then MC DNA was produced and purified by using an MC-Esay™ Kit (System Biosciences), without the optional dNTP removal step.

### Results

The mini-circle (minicircle, MC) vector is a recently developed non-viral strategy that has shown significant improvement compared to conventional plasmid vector—specifically, the SB MC delivery of a CAR transgene has been proven to be more effective and less toxic compared with SB plasmid gene transfer. a head-to-head comparison of the MAJESTIC system with both MC and plasmid DNA systems was performed, using CD3 T cells as a source. 7-AAD staining data of SB/SB100X plasmid DNA and MC-SB/MC-SB100X groups showed similar cell viability (˜70%), with MC-SB+SB100X mRNA group showing slightly higher cell viability. In comparison, AAV-SB-CD22.CAR+SB100X mRNA group (MAJESTIC) attained 86% viability, which was higher than SB/SB100X plasmid DNA, MC-SB/MC-SB100X, and MC-SB+SB100X mRNA groups. CAR-T ratio on day 4 after electroporation confirmed higher CAR-T production efficiency compared of minicircle vs. plasmid DNA electroporation (19.7% vs. 8.01%). Efficiency could be further improved if MC-SB was electroporated with transposase supplied as SB100x mRNA (24.4%) (FIG. 17A). In comparison, the MAJESTIC (AAV-SB-CD22.CAR+SB100X mRNA) group yielded the highest CAR-T ratio (51.8%) at day 4, substantially higher than those of MC/MC-transposase, MC/mRNA-transposase, and transposon plasmids (FIG. 17A).

To further verify these results, another independent set of MC vs MAJESTIC comparisons was performed in an independent human donor, using CD4 and CD8 T cells separately in this case. Flow cytometry revealed CD22.CAR T ratios of 16.1%, 20.0%, and 24.2% for the MC-SB+SB100X mRNA group in donor 6760 CD4 T cells at day 3, 8, and 14, respectively (FIG. 9C). The MAJESTIC group showed CD22.CAR T ratios of 43.4%, 60.9%, and 81.9% in donor 6760 CD4 T cells at day 3, 8, and 14, respectively (FIG. 9C). Similar results were also observed in donor 6760 CD8 T cells. The MC-SB+SB100X mRNA group showed CD22.CAR T ratios of 29.1%, 29.4%, and 37.0% at day 3, 8, and 14, respectively (FIG. 9D), and the AAV-SB-CD22.CAR+SB100X mRNA group showed CD22.CAR T ratios of 60.4%, 76.3%, and 82.4% at day 3, 8, and 14, respectively (FIG. 9D). The yield of MAJESTIC and MC/mRNA-transposase is shown from aggregated replicates (FIGS. 17B-17C).

To further test the variability of the MAJESTIC platform, this technology was applied along with other systems to four new healthy donors (Donor 0007, Donor 4003, Donor 5003, Donor 003C) in a head-to-head comparison manner. Consistently with the results above, across all four donors, the CAR-T ratio was the highest in MAJESTIC group (AAV-SB-CD22.CAR+SB100x mRNA) compared with other groups including MC/MC and MC/mRNA-Transposase (FIG. 9F). Specifically, the CD22.CAR % was on average 28.5% on day 3, and as high as 73.3% on day 21 for MAJESTIC (FIG. 9F). Importantly, although there is donor-to-donor variability as expected, in each respective donor, MAJESTIC is consistently the group with highest efficiency in matched comparisons, across each donor and in all time points (FIG. 9F). Electroporation involving either the plasmid or the MC form of DNA appears to have lower viability and CAR % vs. MAJESTIC even in different cell types and donors (FIG. 9D-9F; FIG. 17A-17C). Together, the data demonstrated the efficiency and reduced cellular toxicity of the MAJESTIC gene transfer platform compared with plasmid and MC gene transfer methods.

### Example 6: Analyzing the Genomic Integration Profile of the MAJESTIC System in CAR-T Cells

To examine the genomic integration profile of the MAJESTIC system in T cells, Splinkerette library preparation was performed followed by next-generation sequencing (NGS) (Methods). CAR-positive T cells were first purified, then isolated genomic DNA from three sets (three independent donors) of MAJESTIC-generated and MC/mRNA-generated CAR-T samples collected d21 after electroporation. The gDNA for these two donors was fragmented and then a two-step Splinkerette PCR conducted to generate insertion site libraries. Analysis of next-generation sequencing data allowed us to map insertion locations in karyograms. These data showed that MAJESTIC indeed mediates cargo integration into the genome of human T cells, across all major chromosomes.

The frequency of insertions into safe harbors was then examined, which are generally defined as regions of the genome where transgene insertions lead to predictable expression and do not interrupt existing gene activity. A list of safe harbor coordinates were used from Querques, et al., Nat Biotechnol, 37, 1502-1512 (2019). Using a random set of 1 million genomic sites, it was estimated that approximately 25% of these random sites intersect with safe harbors. Using this value as a reference point, MAJESTIC was compared to other gene-transfer methods, using integration profile data from literature for lentivirus transduction. To understand trends in the safety profile of MAJESTIC compared to other methods, MAJESTIC-mediated safe harbor insertion frequencies were found to be similar to that for MC/mRNA (around 17% vs. 15% on average). Importantly, MAJESTIC-mediated insertions were much more likely to be within safe harbors compared to that of lentivirus (around 2-3%) (FIG. 9G). Furthermore, the proportion of insertions into functional gene regions were determined, including exons, introns, and cancer genes and calculated the frequencies as fold-change relative to the randomly generated sites (FIG. 9H). Using this functional-gene region profile as a proxy for insertional safety, MAJESTIC mediated a reasonably favorable safety profile, comparable to that of MC/mRNA and better than that of the lentiviral vector (FIG. 9G-9G). Together, these data reveal the integration site profile of the MAJESTIC system and demonstrate a trend towards safer insertions compared to lentiviral transduction.

The functional gene region data shows differences in the frequencies of integration into exons, coding exons, and 5′ UTRs in MC-SB+SB100X mRNA group as compared with a previous study. This may be due to technical reasons, e.g., different sample preparation, different time points used). For example, unlike a previous study which used unselected bulk samples, CAR+ cell population in our workflow were selected, which may explain the elevated exon integration. CAR+ selection enriches for T cells that have the CAR transgene inserted in genomic loci that avoid transgene silencing. Additionally, integration profile differences were observed between MAJESTIC and MC systems, despite the fact that both use the same SB transposon. While differences here could be due to technical reasons (e.g., sample prep) and/or biological reasons associated with the differences in transposon delivery approaches, the cause of which is beyond the scope of this study.

From the reads mapped to the human genome, the data demonstrated that, as expected, SB transposons integrate at genomic loci with a TA motif (Table 3).

TABLE 3
Representative transposon integration sites

GATACAGTGCACATGTGGTACAGTTGAAGTCGGAAGTTTA
(SEQ ID NO: 1)
ATCTCAAAATAGTAAATGCTACAGTTGAAGTCGGAAGTTTA
(SEQ ID NO: 2)
AGGTGACTGATACCAAAAATACAGTTGAAGTCGGAAGTTTA
(SEQ ID NO: 3)
ATAGTACAAAGAGTTCTCATATACAGTTGAAGTCGGAAGTTTA
(SEQ ID NO: 4)
AGACAGACCTACAAAGAATATACAGTTGAAGTCGGAAGTTTA
(SEQ ID NO: 5)
GCAAACCAAAATGGCACATGTACAGTTGAAGTCGGAAGTTTA
(SEQ ID NO: 6)
TATTATCAATAGCACCTAATCTACAGTTGAAGTCGGAAGTTTA
(SEQ ID NO: 7)
AAATTTCTAGAAAAGGGTTGGTACAGTTGAAGTCGGAAGTTTA
(SEQ ID NO: 8)
AATGATTATGGCATTCATATATACAGTTGAAGTCGGAAGTTTA
(SEQ ID NO: 9)
CCAGACTTGGTGGCACACACCTACAGTTGAAGTCGGAAGTTTA
(SEQ ID NO: 10)
AAGAGCTTTTATTTACATGAACTACAGTTGAAGTCGGAAGTTTA
(SEQ ID NO: 11)
CGGAACGTGTAGGTTCGTTACATACAGTTGAAGTCGGAAGTTTA
(SEQ ID NO: 12)
AATCCTAGAACTGGAAAATATAGTACAGTTGAAGTCGGAAGTTTA
(SEQ ID NO: 13)
CTGTGAGTGTGGACTGATCAAATATACAGTTGAAGTCGGAAGTTTA
(SEQ ID NO: 14)
ATGACTGTGTCTGCACCTCTATCTACAGTTGAAGTCGGAAGTTTA
(SEQ ID NO: 15)
AGACCCCATATCTCACACCATACATACAGTTGAAGTCGGAAGTTTA
(SEQ ID NO: 16)
AGACCCCATATCTCACACCATACATACAGTTGAAGTCGGAAGTTTA
(SEQ ID NO: 17)
CAAGACCTAGGCCATGCAAGACATACAGTTGAAGTCGGAAGTTTA
(SEQ ID NO: 18)
GGAATCTCTTTTTCTAATTATTGCTACAGTTGAAGTCGGAAGTTTA
(SEQ ID NO: 19)

The Genomic sequence is indicated in text and the SB transposon (IR/DR) sequence is indicated in bold.

Quantitative analysis revealed that the amount of normalized detected chimeric reads, as an indicator of level of integration, is ˜100× higher in the MAJESTIC-treated T cells (receiving both AAV-SB transduction and mRNA electroporation), as compared to the background (AAV-SB transduction alone, or PBS treated) (FIGS. 14A-B). These data confirmed the genomic integration of the SB transposon in MAJESTIC-treated T cells.

### Example 7: Application of MAJESTIC Across Multiple Human Immune Cell Types Methods

While CAR-Ts have strong clinical potential, they also have inherent limitations. It is well-known that the suppressive tumor microenvironment of solid tumors creates significant hurdles for T cells (Hou, et al., Nat Rev Drug Discov 20, 531-550, doi:10.1038/s41573-021-00189-2 (2021)). However, unlike T cells, myeloid cells such as macrophages and monocytes naturally infiltrate tumors (Dolgin, Cancer-eating immune cells kitted out with CARs. Nat Biotechnol 38, 509-511, doi:10.1038/s41587-020-0520-5 (2020)). In addition, natural killer (NK) cells have been explored as an alternative to T cells for immunotherapy, because they utilize a different set of signaling pathways, have rapid activation despite being innate immune cells, can exhibit TCR-independent cytotoxicity, and are relatively simpler to develop into an off-the-shelf product (Marofi, et al. CAR-NK Cell: A New Paradigm in Tumor Immunotherapy. Front Oncol 11, 673276, doi:10.3389/fonc.2021.673276 (2021)). Therefore, it is of interest to expand cell therapy to other immune cell types, such as NK cells and myeloid cells to overcome the inherent limitations of T cell based therapy. (Bailey, et al., Gene editing for immune cell therapies. Nat Biotechnol 37, 1425-1434, doi:10.1038/s41587-019-0137-8 (2019)).

To test the utility of MAJESTIC in other immune cell types, MAJESTIC was used for delivery of CAR transgenes to generate CAR-NKs, CAR-Monocytes (CAR-Monos) and CAR-Macrophages (CAR-Mas). Lentivirus transduction, and/or plasmid electroporation were again performed in parallel. NK92 is an immortalized NK cell line that has been used to produce CAR-NKs which have achieved use in clinical trials (Tang, X. et al. First-in-man clinical trial of CAR NK-92 cells: safety test of CD33-CAR NK-92 cells in patients with relapsed and refractory acute myeloid leukemia. Am J Cancer Res 8, 1083-1089 (2018)).

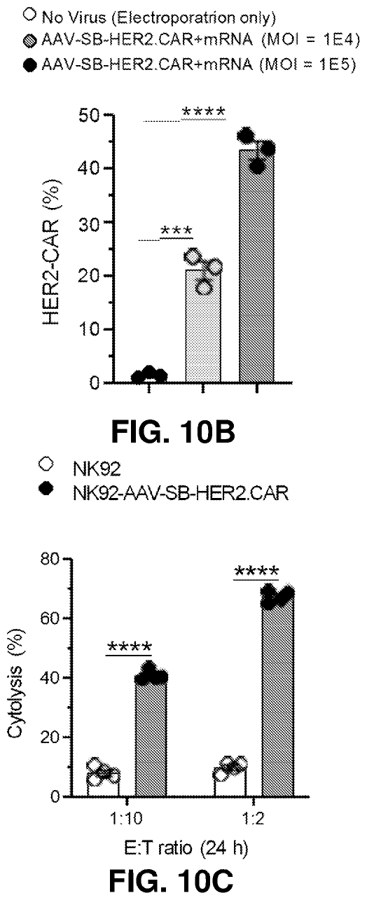

NK92 cells were transduced to engineer HER2-specific CAR-NK cells. Flow cytometry revealed that the MAJESTIC system efficiently generated HER2 CAR-NK cells (near 50% at high dose on day 14), which was significantly higher than those by lentiviral or DNA transposon electroporation in the conditions tested (FIG. 10A). For example, at day 4, Lenti-HER2.CAR at a high MOI (MOI=2.5) yielded 13.9% HER2.CAR-positive NK92 cells, but only 2.71% with low MOI (MOI=1). Plasmid DNA transposon electroporation groups achieved 16.1% and 8.06% HER2.CAR-positive NK92 cells. Strikingly, AAV-SB-HER2.CAR+mRNA groups yielded 41.6% and 13.2% HER2.CAR-positive NK92 cells in high (1E5) and low (1E4) MOI conditions, respectively. Notably, the proportion of the HER2.CAR-positive population did not decline in long-term cultures of MAJESTIC-generated CAR-NK cells: the percentage of HER2.CAR+% NK cells was 44.9-49.0% at day 14 and sustained at 43.8% day 33 (FIG. 10D). Of note, dose-dependence is strong for CAR-NK generation via MAJESTIC, as high (1E5) MOI resulted in significantly higher CAR+% NK cells than low (1E4) MOI (FIG. 10B). Because NK cells can kill cancer cells independent of cancer-specific antigen, the function of the resultant HER2.CAR-NK cells was tested in a kill assay with co-culture of MCF-7 breast cancer cells.

Results showed that the HER2.CAR-NK cells are efficient killer cells, demonstrating significantly stronger killing compared to untreated NK92 cells, with around 70% target cell death 24 h post co-culture (FIG. 10C). These data demonstrate that the MAJESTIC system can be used to efficiently generate stable and functional CAR-NK cells.

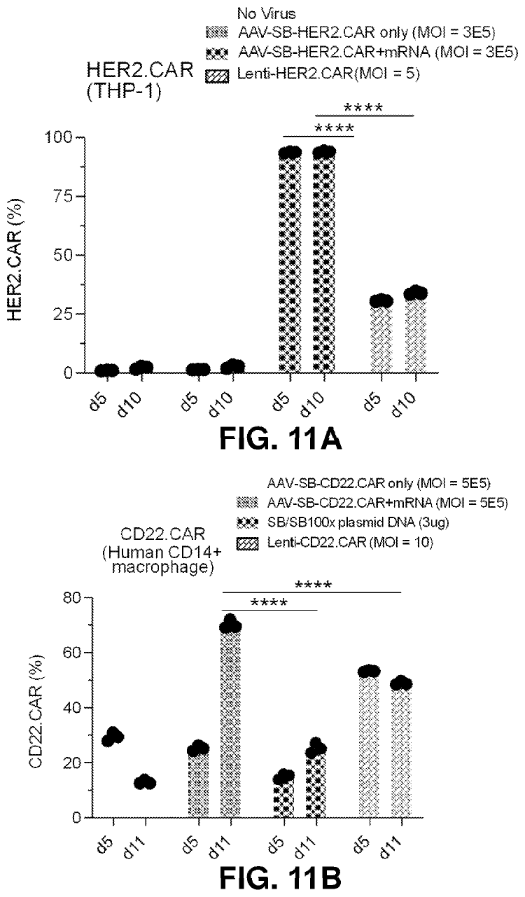

The system was also tested for CAR delivery to THP-1 cells, a human monocytic and myeloid cell line (Auwerx, The human leukemia cell line, THP-1: a multifacetted model for the study of monocyte-macrophage differentiation. Experientia 47, 22-31, doi:10.1007/BF02041244 (1991)). Over 93% HER2.CAR-positive THP-1 with AAV-SB-HER2 (MOI 3E5) on day 5 and 10, compared to ˜30% with lentiviral transduction at MOI 5 (FIG. 11A). For the DNA transposon plasmid electroporation group, cell viability was extremely low, with the vast majority of cells (>99%) dead due to the high cellular toxicity of plasmid electroporation, making it impossible to generate sufficient cells for subsequent analysis.

CAR-MAs were engineered using primary human CD14⁺ macrophages, again comparing MAJESTIC with lentivirus and DNA transposon systems. Flow cytometry revealed a 25.5% CD22-specific CAR-MA population for the AAV-SB-CD22.CAR+mRNA group, which increased to 69.1% by day 11 (FIG. 11B). Lentiviral transduction worked reasonably well in primary macrophages, with CAR-positive percentages of 52.9% on day 5 and 48.1% on day 11 (FIG. 11B). Of note, AAV-SB-CAR alone without the mRNA-transposase yielded nearly 30% CAR+ MAs by day 5, which fell by more than half by day 11 (FIG. 11B). At day 11, the CAR+% of CAR-MAs in the MAJESTIC group is highest among all groups tested (with AAV-SB-CAR alone, lentivirus and DNA transposon). These data suggest that the MAJESTIC system can be used to efficiently generate stable CAR-MA cells.

Finally, because human iPSCs can be used as a source for derivation of various types of cellular lineages, MAJESTIC was applied to iPSCs. Results showed MAJESTIC can transduce iPSCs carrying a HER2.CAR transgene at high efficiency (>75% HER2.CAR+) (FIG. 13D). Along with MAJESTIC, lentiviral vector can also transduce iPSCs at high efficiency, but not transposon DNA electroporation (FIG. 13D). Although differentiation of iPSCs into other cell types takes additional time, Delivery of cell therapy transgenes into iPSCs by MAJESTIC provides another versatile means to generate various therapeutic immune cells efficiently.

Altogether, these data demonstrated that the MAJESTIC system is capable of efficiently engineering stable functional therapeutic immune cells, and is applicable to various types of transgenes and across multiple lineages of immune cells.

### Discussion

Adoptive cell therapy, most notably CAR T therapy, has demonstrated clinical success in patients with several indications of hematological. A vital and potentially limiting step of this therapy is the manufacturing of engineered immune cells: to produce sufficient therapeutic cells for therapy would ideally require 1) a sizeable pool of patient immune cells to begin with and 2) a highly efficient genetic engineering technology. Although low transfer efficiency can be alleviated in part by increased culturing time, high initial efficiency would expedite the manufacturing process. Additionally, increasing culturing time necessitates extended exposure of the cells to their target antigens during production, which may shift cells in favor of a differentiated phenotype, reducing long-term memory function (Morgan, et al., Genetic Modification of T Cells. Biomedicines 4, doi:10.3390/biomedicines4020009 (2016)) and thus the overall quality of the engineered cells. γ-retroviral vectors are indeed capable of efficient genome integration, but their tendency to insert into promoters of actively transcribed genes raises concerns about potential genotoxicity. Lentiviruses are commonly used in clinical trials today, and recent advances have improved their safety. However, there are still certain safety risks associated with the pathogenic origin of such viruses.

The field has also developed various alternative gene transfer methods that can be worked with at BSL-1. AAV is a commonly used gene therapy vector, however, due to the dilution effect, AAV-transduced T cells will have gradual reduction in transgene expression, making an AAV-only system not ideal for delivery of CAR transgenes into T cells. Electroporation of 1) DNA transposon/transposase or 2) CAR-encoding mRNA construct are two non-viral gene transfer strategies, but key limitations include low viability/high toxicity for the former, and transient transgene expression for the latter.

The notable success of immune cell-based cell therapies has invited the introduction of other technologies to enhance immune cell engineering. CRISPR is one such technology that is being rapidly and broadly applied to immune cell editing (Roth, et al. Reprogramming human T cell function and specificity with non-viral genome targeting. Nature 559, 405-409, doi:10.1038/s41586-018-0326-5 (2018)). Such strategies rely on Cas9, Cas12a/Cpf1 or other DNA-targeting endonucleases to generate DSBs, which is then repaired with a donor template via homology-directed repair (HDR). The efficiency of these knock-in/knock-out systems is thus dependent on two steps: 1) the efficiency of enzymatic gene knockout and 2) the rate of incorporation of the homology template. As to 1), knockout efficiency depends on the availability of an optimal guide because poorly designed guides may not cut efficiently and may cause undesired off-target effects (Fu, et al. High-frequency off-target mutagenesis induced by CRISPR-Cas nucleases in human cells. Nat Biotechnol 31, 822-826, doi:10.1038/nbt.2623 (2013); Anderson, et al. CRISPR off-target analysis in genetically engineered rats and mice. Nat Methods 15, 512-514, doi:10.1038/s41592-018-0011-5 (2018); Frock, et al. Genome-wide detection of DNA double-stranded breaks induced by engineered nucleases. Nat Biotechnol 33, 179-186, doi:10.1038/nbt.3101 (2015); Tsai, et al. GUIDE-seq enables genome-wide profiling of off-target cleavage by CRISPR-Cas nucleases. Nat Biotechnol 33, 187-197, doi:10.1038/nbt.3117 (2015); Cameron, et al. Mapping the genomic landscape of CRISPR-Cas9 cleavage. Nat Methods 14, 600-606, doi:10.1038/nmeth.4284 (2017); Tsai, et al. CIRCLE-seq: a highly sensitive in vitro screen for genome-wide CRISPR-Cas9 nuclease off-targets. Nat Methods 14, 607-614, doi:10.1038/nmeth.4278 (2017)).

In addition, CRISPR knockout generates exposed DSBs that may trigger mutagenic non-homologous end-joining (NHEJ) pathways rather than HDR, which is especially hazardous for off-target editing, when no repair template is available for HDR to occur. As to 2), HDR is limited to the late S and G2 phases of the cell cycle (Zhang, et al. Hybrid adeno-associated viral vectors utilizing transposase-mediated somatic integration for stable transgene expression in human cells. PLoS One 8, e76771, doi:10.1371/journal.pone.0076771 (2013)), restricting the interval in which the second step can occur. In any gene editing system, regardless of the enzyme of choice, as long as DSB occurs, two major risks must be considered—the triggering of the p53 pathway, and the possibility of chromosome alterations, which increases with the number of DSBs.

Unlike γ-retroviruses, SB reduces the likelihood of genotoxicity as studies have shown that this class of transposons has close to a random genomic integration profile However, introduction of the SB system into cells by DNA transfection or electroporation can lead to higher cellular toxicity. During remobilization, SB can leave behind a tri-nucleotide footprint; thus, continuous remobilization of the transposon is a potential limitation of an all-in-one AAV-SB system, where both the transposon and transposase are delivered by AAV. The described platform addresses this issue by separating the transposase into a transient delivery component (mRNA). The SB system has also been engineered in the form of combinations or hybrid vectors, e.g., dCas9-SB100X to retarget SB transposition, and an adenovirus-SB hybrid system to achieve higher transduction efficiency. The MAJESTIC system differs from all such efforts by combining the advantages of all three delivery vehicles (AAV, transposon, mRNA) in an organic way: transducing cells with the hybrid AAV-transposon vector with electroporation of transposase mRNA. AAV-SB transduction retains the benefits of high cell viability and stable transgene expression. The process of gene transfer of the MAJESTIC system is similar to conventional SB nucleofection, with mRNA electroporation instead of plasmid or MC electroporation and an extra AAV transduction step in which virus is added directly into the media. From the data, it appears that approaches involving DNA electroporation including plasmid transposon, MC/MC and/or MC/mRNA, naturally have an associated impact on cell viability and yield. MAJESTIC avoids introducing double-stranded, circular DNA into cells and instead uses AAV and mRNA, both of which have reasonably low cellular toxicity.

By virtue of relying on AAV for gene-transfer, the MAJESTIC system will be limited by AAV's packaging size of ˜4.75 kb (˜4.3 kb without SB arms). This is usually sufficient to include the CAR construct and additional elements (e.g., iCasp9), but will face challenges with significantly larger transgenes, which could be accommodated with DNA transposon plasmid/MC systems as transposons can in principle carry large transgene cargos although the efficiency may drop as the size increases. Additionally, given that MAJESTIC is a composite system, the generation of therapeutic immune cells is a two-step process including electroporation/nucleofection+viral transduction, although they can be streamlined to be performed at the same period of time (as demonstrated in our 0 h transduction/electroporation experiments); while lentivirus or plasmid electroporation are both one-step methods. Also, compared to lentivirus or plasmid production, the good manufacturing practice (GMP) production cost of MAJESTIC will be higher because of the requirement for both AAV and mRNA. Without considering yield, MAJESTIC, KIKO, AAV and lentiviral/retroviral approaches all have higher GMP cost as compared to non-viral approaches such as transposon/MC, which is more economic to manufacture per today's GMP landscape (Table 4). Also, MAJESTIC itself cannot achieve precisely targeted gene editing as CRISPR; rather, its advantage is being a high-efficiency gene-editing-free delivery approach. MAJESTIC is an alternative cargo delivery and therapeutic cell generation strategy with the strength of producing CAR+ cells with high viability at high yield—and thus the MAJESTIC system is effective where viability and/or yield is particularly important.

The differences, including the advantages and limitations of the MAJESTIC system are summarized and compared to existing approaches for therapeutic cell engineering in Table 4. MAJESTIC does not replace nor diminish other methods; instead, it provides a novel alternative gene delivery technology that offers superiority and advantages in certain feature areas, such as high viability, efficiency and yield, with naturally associated with limitations such as cargo size, additional procedures, and cost. The versatility of the MAJESTIC system makes it not limited to application in cell therapy for cancer—any therapy or research effort utilizing engineered immune cells could, in principle, benefit by using this system. MAJESTIC can, for example, generate CAR-Ts from cancer patient-derived T cells in a more clinically relevant scenario. Further improvements in the safety of the platform can be achieved, using other components including different AAV serotypes, other viral vectors, or different transposon systems such as a high-soluble Sleeping Beauty transposase (hsSB). Given the modular nature of the MAJESTIC platform, other cargos and cell types can be tested in a broad range of applications by users from various fields.

TABLE 4
Comparison of cell engineering tools

| Gene | | Cellular | | Genomic | p53 | Duration of | |
|---|---|---|---|---|---|---|---|
| delivery | Vehicle | toxicity | Geno- | DSB | pathway | transgene | Transgene |
| systems | class | (ex vivo) | toxicity | generation | trigger | expression | silencing |
| Retrovirus | Viral | Low | Medium | No | No | Stable | Frequent |
| Lentivirus | Viral | Low | Medium | No | No | Stable | Frequent |
| AAV | Viral | Low | Low | No | No | Transient | Infrequent |
| | | | | | | or Stable | |
| Transposon | Non-viral | High | Low | No | No | Stable | Infrequent |
| (DNA) | | | | | | | |
| mRNA | Non-viral | Low | Low | No | No | Transient | Infrequent |
| Cas9 RNP + | Non-viral | Medium - | Medium - | Yes | Likely | Stable | Infrequent |
| DNA donor | | High | High | | | | |
| KIKO (AAV + | Composite | Low | Low | Yes | Likely | Stable | Infrequent |
| Cpfl mRNA) | | | | | | | |
| MAJESTIC | Composite | Low | Low | No | No | Stable | Infrequent |
| (mRNA; | | | | | | | |
| AAV-SB) | | | | | | | |
| (this study) | | | | | | | |

| Gene | | | | Tropism | | | Gene |
|---|---|---|---|---|---|---|---|
| delivery | Integration | Integration | | (Targetable | | Viral | therapy |
| systems | efficiency | preference | Transduction | cell types) | Biosafety | titer | strategy |
| Retrovirus | Medium | TSS | Dividing | Various, by | BSL- | Low | Mostly |
| | | | cells only | pseudotype | 2/2+ | | Ex vivo |
| Lentivirus | Medium | Random | Non-dividing | Various, by | BSL- | Low | In vivo, |
| | | | or dividing | pseudotype | 2/2+ | | Ex vivo |
| | | | cells | | | | |
| AAV | Non- | Rare, but | Non-dividing | Various, by | BSL-1 | High | In vivo, |
| | integrating | strong | or dividing | serotype | | | Ex vivo |
| | (with rare | (AAVS1) | cells | | | | |
| | events) | | | | | | |
| Transposon | Medium | Random | Non-dividing | Limited | BSL-1 | — | Mostly |
| (DNA) | | | or dividing | | | | Ex vivo |
| | | | cells | | | | |
| mRNA | Non- | NA | Non-dividing | Various | BSL-1 | — | In vivo, |
| | integrating | | or dividing | | | | Ex vivo |
| | | | cells | | | | |
| Cas9 RNP + | Medium - | Targeted | Dividing | Various | BSL-1 | NA | In vivo, |
| DNA donor | High | | cells only | | | | Ex vivo |
| KIKO (AAV + | High | Targeted | Dividing | Various | BSL-1 | High | In vivo, |
| Cpfl mRNA) | | | cells only | | | | Ex vivo |
| MAJESTIC | High | Random | Non-dividing | Various, by | BSL-1 | High | In vivo, |
| (mRNA; | | | or dividing | serotype | | | Ex vivo |
| AAV-SB) | | | | | | | |
| (this study) | | | cells | | | | |

In Table 4, DSB: double-stranded break; GMP: Good Manufacturing Practice; RNP: ribonucleoprotein; BSL: biosafety level; KIKO: homology-directed-repair knock-in and immune-checkpoint knockout; TSS: transcription start site.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a nucleic acid sequence is disclosed and discussed and a number of modifications that can be made to a number of molecules including the nucleic acid sequence are discussed, each and every combination and permutation of the nucleic acid sequence and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a”, “an”, and “the” include plural reference unless the context clearly dictates otherwise. Thus, for example, reference to “a nucleic acid sequence” includes a plurality of such nucleic acids, reference to “the nucleic acids” is a reference to one or more nucleic acid and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicate an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated, and considered disclosed is the range from the one particular value and/or to the other particular value unless the context specifically indicates otherwise. Similarly, when values are expressed as approximations, by use of the antecedent “about,” it will be understood that the value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. All of the individual values and sub-ranges of values contained within an explicitly disclosed range are also specifically contemplated and should be considered disclosed unless the context specifically indicates otherwise. Finally, all ranges refer both to the recited range as a range and as a collection of individual numbers from and including the first endpoint to and including the second endpoint. In the latter case, any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives. Thus, for example, a list of different gene targets does not indicate that the listed gene targets are obvious one to the other, nor is it an admission of equivalence or obviousness.

Every component disclosed herein is intended to be and should be considered to be specifically disclosed herein. Further, every subgroup that can be identified within this disclosure is intended to be and should be considered to be specifically disclosed herein. As a result, it is specifically contemplated that any component, or subgroup of components can be either specifically included for or excluded from use or included in or excluded from a list of components.

Those skilled in the art will recognize, or be able to ascertain using no more than routine experimentation, many equivalents to the specific embodiments of the method and compositions described herein. Such equivalents are intended to be encompassed by the following claims.

1. A method of introducing a gene of interest into a cell, the method comprising introducing to the cell:
(i) a viral vector comprising a transposon encoding the gene of interest; and
(ii) mRNA comprising a sequence that encodes one or more transposase enzymes configured to specifically mediate targeted integration of the transposon into the cellular genome,
wherein the mRNA is introduced to the cell via electroporation.

2. The method of claim 1, wherein the transposon is the Sleeping Beauty transposon.

3. The method of claim 2, wherein the transposase enzyme is the Sleeping Beauty SB100x hyperactive transposase.

4. The method of claim 1, wherein the viral vector is an Adeno-associated virus (AAV) vector.

5. The method of claim 1, wherein the transposon comprises a gene of interest comprising a reporter gene, a Chimeric Antigen Receptor (CAR), or combinations thereof.

6. The method of claim 5, wherein the transposon further comprises a promoter and/or polyadenylation signal operationally linked to the reporter gene and/or the CAR.

7. The method of claim 5, wherein the CAR is specific for an antigen selected from the group consisting of a cancer antigen, an inflammatory disease antigen, a neuronal disorder antigen, HIV/AIDS, a diabetes antigen, a cardiovascular disease antigen, an infectious disease antigen (including a viral antigen, a protozoan antigen, a bacterial antigen, and an allergen), an autoimmune disease antigen and an autoimmune disease antigen, or combinations thereof.

8. The method of claim 5, wherein the CAR targets one or more antigens selected from the group consisting of AFP, AKAP-4, ALK, Androgen receptor, B7H3, BCMA, Bcr-Abl, BORIS, Carbonic, CD123, CD138, CD174, CD19, CD20, CD22, CD30, CD33, CD38, CD80, CD86, CEA, CEACAM5, CEACAM6, Cyclin, CYP1B1, EBV, EGFR, EGFR806, EGFRvIII, EpCAM, EphA2, ERG, ETV6-AML, FAP, Fos-related antigen1, Fucosyl, fusion, GD2, GD3, GloboH, GM3, gp100, GPC3, HER-2/neu, HER2, HMWMAA, HPV E6/E7, hTERT, Idiotype, IL12, IL13RA2, IM19, IX, LCK, Legumain, lgK, LMP2, MAD-CT-1, MAD-CT-2, MAGE, MelanA/MART1, Mesothelin, MET, ML-IAP, MUC1, Mutant p53, MYCN, NA17, NKG2D-L, NY-BR-1, NY-ESO-1, NY-ESO-1, OY-TES1, p53, Page4, PAP, PAX3, PAX5, PD-L1, PDGFR-0, PLAC1, Polysialic acid, Proteinase3 (PR1), PSA, PSCA, PSMA, Ras mutant, RGS5, RhoC, ROR1, SART3, sLe(a), Sperm protein 17, SSX2, STn, Survivin, Tie2, Tn, TRP-2, Tyrosinase, VEGFR2, WT1, and XAGE.

9. The method of claim 7, wherein the antigen is a cancer antigen selected from the group consisting of 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD 152, CD 19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DR5, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-0, TRAIL-R1, TRAIL-R2, tumor antigen CTAA16.88, VEGF-A, VEGFR-1, VEGFR2, and vimentin.

10. The method of claim 5, wherein the CAR is bispecific or multivalent.

11. The method of claim 5, wherein the CAR is anti-CD19 or anti-CD22, or both.

12. The method of claim 11, wherein the CAR is CD19BBz or CD22BBz.

13. The method of claim 1, wherein the mRNA comprises N6-methyladenosine (m6A), 5-methylcytosine (m5C), pseudouridine (W), N1-methylpseudouridine (me1ψ), 5-methoxyuridine (5moU), a 5′ cap, a poly(A) tail, one or more nuclear localization signals, or combinations thereof.

14. The method of claim 1, wherein the mRNA, or the transposon, or both are codon optimized for expression in a eukaryotic cell.

15. The method of claim 1, wherein the mRNA encoding transposase and the viral vector is introduced to the cell at the same or different times.

16. The method of claim 15, wherein the mRNA is introduced to the cell by electroporation at a time point between 10 hours before, and 10 hours after the viral vector comprising a transposon encoding the gene of interest is introduced to the cell.

17. The method of claim 16, wherein the mRNA is introduced to the cell by electroporation at a time point between one and four hours before the viral vector comprising a transposon encoding the gene of interest is introduced to the cell.

18. The method of claim 4, wherein the AAV vectors is AAV6 or AAV9.

19. The method of claim 1, wherein the introduction is performed ex vivo.

20. The method of claim 1, wherein the cell is a T cell, hematopoietic stem cell (HSC), macrophage, natural killer cell (NK), or dendritic cell (DC).

21. The method of claim 20, wherein the T cell is a CD8+ T cell selected from the group consisting of effector T cells, memory T cells, central memory T cells, and effector memory T cells.

22. The method of claim 21, wherein the T cell is a CD4+ T cell selected from the group consisting of Th1 cells, Th2 cells, Th17 cells, and Treg cells.

23. An isolated cell modified according to the method of claim 1.

24. The isolated cell of claim 23, wherein the cell includes a gene of interest that is a CAR.

25. The isolated cell of claim 24, wherein the CAR is bispecific or multi-specific.

26. A population of cells derived by expanding the cell of claim 23.

27. A pharmaceutical composition comprising the population of cells of claim 26 and a pharmaceutically acceptable buffer, carrier, diluent or excipient.

28. A method of treating a subject having a disease, disorder, or condition comprising administering to the subject an effective amount of the pharmaceutical composition of claim 27.

29. A method of treating a subject having a disease, disorder, or condition associated with an elevated expression or specific expression of an antigen, the method comprising administering to the subject an effective amount of a T cell modified according to the method of claim 5, wherein the T cell comprises a CAR that targets the antigen.

30. A method of treating a subject having a disease, disorder, or condition, the method comprising administering to the subject an effective amount of a pharmaceutical composition comprising a genetically modified cell, wherein the cell is genetically modified by the method of claim 1.

31-46. (canceled)

47. The method of claim 30, wherein the transposon comprises a gene of interest comprising a reporter gene, a chimeric antigen receptor (CAR), or combinations thereof, wherein the introduction to the cell is performed ex vivo, wherein the cell was isolated from the subject having the disease, disorder, or condition prior to the introduction to the cell.

48. The method of claim 30, wherein the transposon comprises a gene of interest comprising a reporter gene, a chimeric antigen receptor (CAR), or combinations thereof, wherein the introduction to the cell is performed ex vivo, wherein the cell was isolated from a healthy donor prior to the introduction to the cell.

49. The method claim 30, wherein the pharmaceutical composition comprises a population of cells derived by expanding the genetically modified cell.

50. The method claim 30, wherein the subject is a human.
